SEQUENCE LISTING (28 sequences)

SEQ ID NO: 1 - DNA, 24 bp, synthetic construct
  source 1..24: mol_type = other DNA
caccgggtttgctgcccggcggac

SEQ ID NO: 2 - DNA, 24 bp, synthetic construct
  source 1..24: mol_type = other DNA
aaacgtccgccgggcagcaaaccc

SEQ ID NO: 3 - DNA, 23 bp, synthetic construct
  source 1..23: mol_type = other DNA
ccggccagcgtggtcatcctggg

SEQ ID NO: 4 - DNA, 23 bp, synthetic construct
  source 1..23: mol_type = other DNA
tgcccccaggatgaccacgctgg

SEQ ID NO: 5 - DNA, 24 bp, synthetic construct
  source 1..24: mol_type = other DNA
ctcgaactcctgaccttgtgatct

SEQ ID NO: 6 - DNA, 25 bp, synthetic construct
  source 1..25: mol_type = other DNA
cacaaggcttactctcacctcttca

SEQ ID NO: 7 - DNA, 24 bp, synthetic construct
  source 1..24: mol_type = other DNA
gacgggacagtttctcatctatct

SEQ ID NO: 8 - DNA, 22 bp, synthetic construct
  source 1..22: mol_type = other DNA
atccctatccacacgtgaacct

SEQ ID NO: 9 - DNA, 23 bp, synthetic construct
  source 1..23: mol_type = other DNA
taacgaggacagggtagctttgt

SEQ ID NO: 10 - DNA, 24 bp, synthetic construct
  source 1..24: mol_type = other DNA
catcttgaccattcagctagaacc

SEQ ID NO: 11 - DNA, 26 bp, synthetic construct
  source 1..26: mol_type = other DNA
ccacagaatgttgtagagttcaatgc

SEQ ID NO: 12 - DNA, 25 bp, synthetic construct
  source 1..25: mol_type = other DNA
ttccacgagagtttatgagagagca

SEQ ID NO: 13 - DNA, 25 bp, synthetic construct
  source 1..25: mol_type = other DNA
ggatgtcctatggaaacctatttca

SEQ ID NO: 14 - DNA, 27 bp, synthetic construct
  source 1..27: mol_type = other DNA
gaaggaactcactgtacagaagacaaa

SEQ ID NO: 15 - DNA, 24 bp, synthetic construct
  source 1..24: mol_type = other DNA
gaagagaccagaacaggagacgtg

SEQ ID NO: 16 - DNA, 25 bp, synthetic construct
  source 1..25: mol_type = other DNA
tttatcataacgcggcctctaattc

SEQ ID NO: 17 - DNA, 23 bp, synthetic construct
  source 1..23: mol_type = other DNA
acatacatgcacacacgcacact

SEQ ID NO: 18 - DNA, 24 bp, synthetic construct
  source 1..24: mol_type = other DNA
gctgaagaggcaagagacagaatg

SEQ ID NO: 19 - DNA, 27 bp, synthetic construct
  source 1..27: mol_type = other DNA
aggagttcagtgctaagaatgtgtcaa

SEQ ID NO: 20 - DNA, 26 bp, synthetic construct
  source 1..26: mol_type = other DNA
tgagtaactcctgtcaccactctgaa

SEQ ID NO: 21 - DNA, 24 bp, synthetic construct
  source 1..24: mol_type = other DNA
gatatcgaccagctgaatccagag

SEQ ID NO: 22 - DNA, 24 bp, synthetic construct
  source 1..24: mol_type = other DNA
tcagcgggaaggactttatgtatg

SEQ ID NO: 23 - DNA, 24 bp, synthetic construct
  source 1..24: mol_type = other DNA
agggaatggatagactggtgtgct

SEQ ID NO: 24 - DNA, 26 bp, synthetic construct
  source 1..26: mol_type = other DNA
actctgcagtcaatacggttaccaaa

SEQ ID NO: 25 - DNA, 23 bp, synthetic construct
  source 1..23: mol_type = other DNA
gtggttcctgcatgaagacagtg

SEQ ID NO: 26 - DNA, 21 bp, synthetic construct
  source 1..21: mol_type = other DNA
ttctgatggcggactttaccg

SEQ ID NO: 27 - DNA, 24 bp, synthetic construct
  source 1..24: mol_type = other DNA
tgaagctgattatgtggcacagtc

SEQ ID NO: 28 - DNA, 24 bp, synthetic construct
  source 1..24: mol_type = other DNA
gacttggatcctggaacgtcatta

(19) United States
(12) Patent Application Publication
(10) Pub. No.: US 2025/0115872 A1
(43) Pub. Date: Apr. 10, 2025

(54) GENERATION OF BROWN ADIPOCYTES FROM HUMAN PLURIPOTENT STEM CELLS

(71) Applicant: The Brigham and Women's Hospital, Inc., Boston, MA (US)

(72) Inventors: Olivier Pourquie, Brookline, MA (US); Jyoti Rao, Brookline, MA (US); Yuchuan Miao, Boston, MA (US)

(21) Appl. No.: 18/834,704

(22) PCT Filed: Feb. 1, 2023
(86) PCT No.: PCT/US2023/061754

§ 371 Date: Jul. 31, 2024

Related U.S. Application Data
(60) Provisional application No. 63/305,390, filed on Feb. 1, 2022.

(51) Int. Cl. C12N 5/077

(52) U.S. Cl. CPC C12N 5/0653; C12N 2501/105; C12N 2501/115; C12N 2501/12; C12N 2501/15; C12N 2501/155; C12N 2501/395; C12N 2501/415; C12N 2501/734; C12N 2506/45

(57) ABSTRACT

Provided herein are methods of generating Gata6-positive brown adipose progenitor cells, and brown adipose cells and tissue therefrom. Additionally, provided herein are methods of generating UCP1 positive organoids. Generally, the methods include a step of culturing brown adipose progenitor cells in a medium containing an FGF signaling pathway activator, a BMP signaling pathway activator, a TGFβ activator, a Wnt inhibitor, and a thyroid hormone receptor activator.

### CLAIM OF PRIORITY

This application claims the benefit of U.S. Provisional Application Ser. No. 63/305,390, filed on Feb. 1, 2022. The entire contents of the foregoing are incorporated herein by reference.

### SEQUENCE LISTING

The instant application contains a Sequence Listing that has been submitted electronically as an XML file named “29618-0376WO1_SL_ST26.xml”. The XML file, created on Jan. 31, 2023, is 25,788 bytes in size. The material in the XML file is hereby incorporated by reference in its entirety.

### TECHNICAL FIELD

Generation of brown adipocytes from human pluripotent stem cells.

### BACKGROUND

Understanding of human brown adipose tissue (BAT) development is limited. Current in vitro models to study human BAT are mostly based on brown adipocytes differentiated in vitro from primary cell lines and stromal vascular cells (Samuelson and Vidal-Puig, 2020). Several protocols have also been established to differentiate human pluripotent stem cells (hPSCs) into brown adipocytes using either transgenic overexpression of transcription factors (Ahfeldt et al., 2012) or treatment with growth factors and small molecules (Takeda et al., 2017). Other reported methods rely on serum-based spontaneous differentiation or treatment of hPSCs with a cytokine cocktail (Guenantin et al., 2017; Hafner et al., 2016; Nishio et al., 2012). Recently, protocols allowing the generation of brown adipocytes from hPSCs by recapitulating developmental cues have also been reported (Carobbio et al., 2021; Zhang et al., 2020). However, to date, a well characterized roadmap of brown fat lineage development is still missing. Therefore, benchmarking these protocols against the normal trajectory of brown adipocytes differentiation in vivo has not been possible.

### SUMMARY

Provided herein are methods of generating a Gata6-positive brown adipocyte precursor cell, the method comprising culturing PAX3-positive somitic progenitor cells in a medium comprising effective amounts of each of an FGF signaling pathway activator, a BMP signaling pathway activator, a TGFβ signaling pathway activator, a Wnt signaling pathway inhibitor, and a thyroid hormone receptor activator under conditions and for a time sufficient for the PAX3-positive cells to differentiate into a Gata6-positive brown adipocyte precursor cell.

Provided herein are methods wherein the PAX3-positive somatic progenitor cells are generated by a method comprising culturing a pluripotent cell, preferably an induced pluripotent stem cell (iPSC) or embryonic stem (ES) cell in a medium comprising effective amounts of each of an hepatocyte growth factor (HGF) signaling pathway activator, an insulin-like growth factor (IGF) signaling pathway activator, and an fibroblast growth factor (FGF) signaling pathway activator, a Wnt signaling pathway activator, and a bone morphogenetic protein (BMP) signaling pathway inhibitor, to generate a PAX3-positive somatic progenitor cell.

Provided herein are methods further comprising culturing the PAX3-positive somatic progenitor cell in a medium comprising an HGF signaling pathway activator and an IGF signaling pathway activator but lacking an FGF signaling pathway activator and a BMP signaling pathway inhibitor for between 8 and 23 days.

In some embodiments, the methods further comprise dissociating the PAX3-positive somatic progenitor cells after culturing the cells between 8 and 23 days. In some embodiments, the HGF signaling pathway activator comprises HGF. In some embodiments, the IGF signaling pathway activator comprises IGF-1. In some embodiments, the FGF signaling pathway activator comprises at least one of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, and combinations thereof. In some embodiments, the FGF signaling pathway activator comprises FGF-2. In some embodiments, the BMP signaling pathway inhibitor comprises at least one of LDN-193189, dorsomorphin, Noggin, Follistatin, Cerberus, and combinations thereof. In some embodiments, the BMP signaling pathway inhibitor comprises LDN-193189. In some embodiments, the BMP signaling pathway activator comprises at least one of BMP2, BMP4, BMP7, and combinations thereof. In some embodiments, the Wnt signaling pathway inhibitor comprises at least one of C59, XAV939, IWR-1, JW74, JW55, G007-LK, MSC2504877, benzimidazolone, WNT974, RK-287107, E7499, IWP-L6, GNF-1331, GNF-6231, ETC-131, IWP-29, LGK947, ETC159, ETC-1922159, and combinations thereof. In some embodiments, the Wnt signaling pathway inhibitor comprises C59. In some embodiments, the TGFβ signaling pathway activator comprises at least one of TGFβ1, TGFβ2, TGFβ3, and combinations thereof. In some embodiments, the TGFβ signaling pathway activator comprises TGFβ1. In some embodiments, the thyroid hormone receptor activator comprises at least one of triiodothyronine (T3), T4, resmetirom, eprotirome, sobetirome, Sob-AM2, VK2809, MB07344, IS25 TG68, and combinations thereof. In some embodiments, the thyroid hormone receptor activator comprises T3. In some embodiments, the HGF signaling pathway activator in some methods is different than the HGF signaling pathway activator in other parts of the same method. In some embodiments, the IGF1 signaling pathway activator in some methods is different than the IGF1 signaling pathway activator in other parts of the same method. In some embodiments, the FGF2 signaling pathway activator in some methods is different than the FGF2 signaling pathway activator in other parts of the same method. In some embodiments, dissociating the PAX3-positive somatic progenitor cells comprises applying one or more of Type IV collagenase and trypsin EDTA. In some embodiments, the methods further comprise seeding the dissociated cells at a density of 60,000-100,000/cm2. In some embodiments, the methods further comprise culturing the Gata6-positive cells in adipogenic differentiation medium to generate brown adipocytes. In some embodiments, adipogenic differentiation medium comprises Dulbecco's Modified Eagle Medium (DMEM) high glucose medium supplemented with serum-free serum replacement, 1× Insulin-Transferrin-Selenium (ITS), isobutylmethylxanthine (IBMX), 1-ascorbic acid, T3, a TGFβ inhibitor, dexamethasone, epidermal growth factor (EGF), hydrocortisone, and rosiglitazone.

Provided herein are in vitro methods of generating a UCP1-positive, Gata6-positive, and/or Pparg positive brown adipocyte organoid, the method comprising culturing a PAX3-positive somite-like structure in a medium comprising effective amounts of each of an FGF signaling pathway activator, a BMP signaling pathway activator, a TGFβ signaling pathway activator, a Wnt signaling pathway inhibitor, and triiodothyronine (T3), under conditions and for a time sufficient for the PAX3-positive somite-like structure to differentiate into a UCP1-positive, Gata6-positive, and/or Pparg positive brown adipocyte organoid.

Provided herein are methods wherein the PAX3-positive somite-like structure is generated by a method comprising culturing a pluripotent cell, preferably an induced pluripotent stem cell (iPSC) or embryonic stem (ES) cell in a medium comprising effective amounts of each of a Wnt activator and a bone morphogenetic protein (BMP) inhibitor, to generate a PAX3-positive somite-like structure.

In some embodiments, the HGF signaling pathway activator comprises HGF. In some embodiments, the IGF signaling pathway activator comprises IGF-1. In some embodiments, the FGF signaling pathway activator comprises at least one of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, and combinations thereof. In some embodiments, the FGF signaling pathway activator comprises FGF-2. In some embodiments, the BMP signaling pathway inhibitor comprises at least one of LDN-193189, dorsomorphin, Noggin, Follistatin, Cerberus, and combinations thereof. In some embodiments, the BMP signaling pathway inhibitor comprises LDN-193189. In some embodiments, the BMP signaling pathway activator comprises at least one of BMP2, BMP4, BMP7, and combinations thereof. In some embodiments, the Wnt signaling pathway inhibitor comprises at least one of C59, XAV939, IWR-1, JW74, JW55, G007-LK, MSC2504877, benzimidazolone, WNT974, RK-287107, E7499, IWP-L6, GNF-1331, GNF-6231, ETC-131, IWP-29, LGK947, ETC159, ETC-1922159, and combinations thereof. In some embodiments, the Wnt signaling pathway inhibitor comprises C59. In some embodiments, the TGFβ signaling pathway activator comprises at least one of TGFβ1, TGFβ2, TGFβ3, and combinations thereof. In some embodiments, the TGFβ signaling pathway activator comprises TGFβ1. In some embodiments, the thyroid hormone receptor activator comprises at least one of triiodothyronine (T3), T4, resmetirom, eprotirome, sobetirome, Sob-AM2, VK2809, MB07344, IS25 TG68, and combinations thereof. In some embodiments, the thyroid hormone receptor activator comprises T3. In some embodiments, the methods further comprise culturing the organoids in adipogenic differentiation medium to generate brown adipocyte organoids. In some embodiments, the adipogenic differentiation medium comprises Dulbecco's Modified Eagle Medium (DMEM) high glucose medium supplemented with serum-free serum replacement, 1× Insulin-Transferrin-Selenium (ITS), isobutylmethylxanthine (IBMX), 1-ascorbic acid, T3, a TGFβ inhibitor, dexamethasone, epidermal growth factor (EGF), hydrocortisone, and rosiglitazone.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-G: FIG. 1A—Uniform Manifold Approximation and Projection (UMAP) embedding showing cell clusters identified using Leiden based clustering on the somitic lineage of the mouse dataset. Colors indicate identified cell cluster. (BApre=brown adipocyte precursors FP=Fibroblasts precursors). FIG. 1B—UMAP embedding of the single cells isolated from the somitic lineage of E11.5, E12.5, E13.5, E14.5 and E15.5 mouse embryos. Colors indicate embryonic days. FIG. 1C—Tracksplot showing the expression of markers of brown adipocyte differentiation in the clusters shown in A. Height of the bars indicated expression level in individual cells. FIG. 1D—Barplot showing the number of cells in indicated clusters at 5 time points. FIG. 1E—STREAM plot showing the putative developmental trajectories inferred by the STREAM algorithm. FIG. 1F—UMAP embedding showing the progression of a biological process from snapshot data using diffusion pseudotime. FIG. 1G—Immunofluorescence analysis of developing interscapular brown fat in mouse. Images represents transverse section of mouse embryos at the forelimb level showing interscapular region. Perilipin (Plin1) expressing adipocytes start to appear on embryonic day (E) 14.5 and perilipin is widely expressed in interscapular fat on E15.5. Staining for Pparg antibody illustrating expression of Pparg starting at E13.5. Interscapular fat develops in between muscle bundles stained using a Myosin heavy chain (Myhc) antibody. NT=Neural tube, n≥3.

FIGS. 2A-E: FIG. 2A—Pie charts showing the time evolution of the proportion of Gata6 positive cells in the clusters indicated on the right. FIG. 2B—Immunofluorescence analysis of developing interscapular fat in mouse from embryonic day (E) 12.5 to 15.5. Images represents transverse section of mouse embryos at the forelimb level showing the interscapular region. Presence of Gata6 positive and Pparg negative cells detected by antibody staining at E12.5. Expression domain of Gata6 expands and double positive cells are detected at E13.5. Expression of Gata6 reduced at E14.5 and only detected in a few cells at E15.5 as majority of cells become Pparg positive. Note expression Gata6 in the hair follicle at E15.5. NT=Neural tube, n≥4. FIG. 2C—Schematic illustrating the lineage tracing experiments in Gata6-CreERT2:Rosa26—tdTomato mice. A single dose of Tamoxifen was injected into pregnant females at E12.5 and embryos were harvested at E15.5. Transverse section at the forelimb level of E15.5 embryos were stained for anti-RFP antibody to detect tdTomato positive cells. Gata6 progeny was detected in the interscapular brown adipose tissue (BAT), the lung (L) and the heart (H). NT=Neural tube (left panel). Magnified image of the transverse section from the interscapular region showing brown adipose tissue stained with Pparg and RFP (tdTomato) antibody (upper right panel). Higher magnification image of brown adipocytes in the interscapular region showing double positive cells for Pparg and tdTomato. Arrowheads mark some of the double positive cells (lower right panel). n≥3. FIG. 2D—Schematic illustrating lineage tracing strategy to label Pax7 progeny during mouse development. Pax7-Cre:Rosa26-mTmG embryos were analyzed at E14.5. Representative image showing Pax7 progeny at E14.5. Transverse section at the forelimb level of E14.5 embryos were stained for anti-GFP antibody to detect membrane GFP positive cells. GFP labelling identifies Pax7 progenies in the interscapular brown adipose tissue and the skeletal muscle bundles (SkM) in the dorsal and limb region. NT=Neural tube (left panel). Transverse section of E14.5 Pax7-Cre:Rosa26-mTmG embryo showing interscapular region stained with antibodies against Pparg and GFP to label brown adipocytes and Pax7 progeny (upper right panel). Representative high magnification image showing double positive cells stained for nuclear Pparg and membranous GFP. Arrowheads mark double positive cells (lower right panel). n≥3. FIG. 2E—Transverse section of E14.5 Pax7-Cre:Rosa26-mTmG embryo showing interscapular region stained with antibody against GFP to label Pax7 progeny and Gata6 to label brown adipocyte precursors. Inset shows magnified view. Arrowheads mark some of the double positive cells. NT=Neural tube, n≥3.

FIGS. 3A-3C: FIG. 3A—Immunofluorescence analysis of human fetal tissues (upper panel—skin, lower panel—dorsal muscles) isolated from scapular region of a 98-day old fetus. Antibody staining for MYHC showing skeletal muscle bundles just below the dermis and internal sections. EBF2 and GATA6 label precursor cells, n≥3 sections. FIG. 3B—Immunofluorescence analysis of human fetal brown adipose tissue isolated from the scapular region of a 98-day old fetus. Immunofluorescence analysis confirming expression of UCP1 and PPARG in the tissue (left panel). Immunofluorescence analysis of consecutive section of human fetal brown adipose tissue from a 98-day old fetus shown in the right panel. Antibody staining showing expression of GATA6 in the brown adipocytes precursors and PPARG expression in the precursors and lipid droplets containing adipocytes (right panel), n≥3 sections. FIG. 3C—Immunofluorescence analysis of human fetal brown adipose tissue isolated from scapular region of a 135-day old fetus. Expression of PLIN1, PPARG, UCP1 (upper panel), and GATA6 (lower panel) detected with specific antibodies, n≥3 sections.

FIGS. 4A-M: FIG. 4A—Schematic describing the steps to differentiate human induced pluripotent stem cells (iPSCs) into presomitic mesoderm (PSM), followed by a somite-like stage and somitic derivatives in a monolayer culture. Cells are replated between day 16-30 of differentiation to generate brown adipocytes. + indicates additional days after replating. FIG. 4B—Venus fluorescence signal in MSGN-Venus knock-in iPSC line on day 2 of differentiation, n=4. FIG. 4C—Immunofluorescence analysis for transcription factor TBX6 on day 2 of differentiation, n=4. FIG. 4D—Venus fluorescence signal in PAX3-Venus knock-in iPSC line on day 8 of differentiation, n=7. FIG. 4E—FACS analysis quantification of PAX3-venus positive cells on day 8 of differentiation. Mean±SD, n=14. FIG. 4F—UMAP embedding showing cell clusters identified using Leiden based clustering on the human in-vitro cultured cells at day 20. Colors indicate identified cell cluster. FIG. 4G—Machine-learning classification of human in-vitro cultured cells and mouse embryo. A k-NN classifier trained on clusters of mouse clusters was used to predict identities of the human in-vitro cultured progenitors. FIG. 4H—Machine-learning classification of human in-vitro cultured cells and mouse embryo. A k-NN classifier trained on clusters of E11.5, E12.5, E13.5, E14.5, E15.5 mouse embryos was used to predict identities of the human in-vitro cultured time points at day 20, 40, and 60. FIG. 4I—Immunofluorescence analysis of transcription factor EBF2 and PAX7 on day 20 of differentiation, n=8. FIG. 4J—UMAP embedding showing expression of a curated list of signaling genes. FIG. 4K—Immunofluorescence analysis for transcription factor GATA6 after replating and culturing of 20-day old cultures in HI medium or adipogenic BCTFT medium for 4 days (+4 represents 4 days after replating). n=5. FIG. 4L—Quantification of GATA6 positive cells in FIG. 4G. Mean±SD, n=5, t-test, p<0.001. FIG. 4M—RT-qPCR analysis for GATA6, PPARG and MYOG on day 20 of differentiation and 4 days after replating in HI medium or adipogenic BCTFT medium (+4 represents 4 days after replating). Mean±SD, n=6-8, t-test, p<0.0001.

FIGS. 5A-M: FIG. 5A—Diagram outlining the targeting strategy used to generate UCP1-mCherry knock-in iPSC line. FIG. 5B—Brightfield and mCherry signal in iPSC-derived brown adipocyte cultures on day 40 after replating, n≥20. FIG. 5C—Quantification of mCherry positive cells in iPSC-derived brown adipocyte cultures on day 40 after replating. Mean±SD, n=7. FIG. 5D-E—Immunofluorescence staining with UCP1 and mCherry antibody in the UCP1-mCherry knock-in iPSC line derived brown adipocyte cultures on day 40 after replating. Fraction of UCP1 and mCherry double positive cells from all mCherry positive cells. n=10. FIG. 5E—Immunofluorescence staining for PLIN1 antibody in the UCP1-mCherry knock-in iPSC line derived brown adipocyte cultures on day 40 after replating. n=6. FIG. 5F—Neutral lipid staining using 0.5 mM BODIPY in iPSC-derived brown adipocyte cultures on day 40 after replating, n=3. FIG. 5G—RT-qPCR analysis of iPSC-derived brown adipocyte cultures on day 40 after replating. Mean±SD, n=11-14. FIG. 5H—Immunofluorescence staining for mitochondria on iPSC-derived brown adipocyte cultures on day 40 after replating, n=5. FIG. 5I—Representative transmission electron micrographs of iPSCs (left) and iPSC-derived brown adipocyte (middle and right) demonstrating ultrastructural characteristics and mitochondrial characteristics, n=3. FIG. 5J-K—Immunofluorescence staining for EBF2 (O), GATA6 (P) in the iPSC-UCP1mCherry knock-in iPSC-derived brown adipocyte cultures on day 40 after replating, n=4. FIG. 5L—Periodic Acid staining on brown adipocyte precursors (day 20) and brown adipocytes (day 40 after replating), n=3. FIG. 5M—Representative transmission electron micrographs demonstrating glycogen accumulation in 40 day old replated iPSC-derived adipocytes, n=3.

FIGS. 6A-H: FIG. 6A—UMAP embedding showing cell clusters identified using Leiden based clustering on the human in-vitro cultured cells at day 60. Colors indicate identified cell cluster. FIG. 6B—Machine-learning classification of human in-vitro cultured cells and mouse embryo. A k-NN classifier trained on clusters of mouse clusters was used to predict identities of the human in-vitro cultured clusters. FIG. 6C—UMAP embedding showing cell clusters identified using Leiden based clustering on the human in-vitro cultured cells at day 20, 40, and 60 merged. Colors indicate identified cell cluster. FIG. 6D—UMAP embedding showing cell time points on the human in-vitro cultured cells at day 20, 40, and 60 merged. Colors indicate culture days. FIG. 6E—Barplot showing the proportions of cell clusters per time points. FIG. 6F—Tracksplot showing the expression level of makers genes of the brown adipocyte lineage in individual cells in the Leiden clusters shown in C (top) and at the different ages as shown in D (bottom). FIG. 6G—Graph showing how probability mass flows from the progenitor cluster to the others as time increases based on waddington-OT transition matrix. FIG. 6H—Graph showing how probability mass flows from the Brown Adipocytes Precursors cluster to the others as time increases based on waddington-OT transition matrix.

FIGS. 7A-E: FIG. 7A-Comparison of tracksplots showing expression of marker genes of the brown adipocyte lineage in the mouse embryo at different stages (left) and in human cultures at different days (right) in the clusters identified in FIG. 1A and FIG. 6C respectively. FIG. 7B—Heatmap showing differentially expressed genes in undifferentiated human pluripotent stem cell (iPSC), human fetal brown adipose tissue (fBAT), 40-day old replated iPSC-derived adipogenic cultures (iPSC-BA) and iPSC derived skeletal muscle (iPSC-SkM, described in Al Tanoury et al. 2021). Scale bar represents log normalized read counts. FIG. 7C—WikiPathway 2019 Human analysis of top 200 differentially expressed genes (FDR<0.05, log 2|FC|>6) in iPSC-BAs compared to undifferentiated iPSCs. FIG. 7D—Estimation of browning probability of iPSC derived brown adipocytes (iPSC-BA), iPSC derived skeletal muscle (iPSC-SkM) and human fetal brown adipose tissue (fBAT) using PROFAT database. Numbers represent replicates. FIG. 7E—Heatmap comparing transcriptomic profiles of iPSC derived brown adipocytes (iPSC-BA), human fetal brown adipose tissue (fBAT), human embryonic stem cell derived brown adipocytes in Zhang et al. 2020 (H9-d50) and iPSC derived brown adipocytes in Carobbio et al. 2021 (KOLF2-C1-d25).

FIGS. 8A-D: FIG. 8A—RT-qPCR analysis for UCP1 on 40 day old replated iPSC-derived adipogenic cultures treated with 10 μM forskolin or vehicle control DMSO treatment for 4 hours. Relative gene expression is shown as relative change to undifferentiated iPSC. Mean±SD, n=5, t-test, p<0.01. FIG. 8B—Measurement of glycerol released in culture medium 40 day old replated iPSC-derived adipogenic cultures treated with 10 μM forskolin or vehicle control DMSO treatment for 4 hours. Glycerol amount normalized using total protein amount. Mean±SD, n=4, t-test, p≤0.01. FIG. 8C—Measurement of fluorescent intensity of thermosensitive ERthermAC dye in iPSC derived precursors on day 20 of differentiation and brown adipocytes after 40 days of replating in response to 10 μM forskolin or vehicle control DMSO. Each data point represents Mean±SD, n=3, t-test, p=0.0009. FIG. 8D—Seahorse analysis to measure oxygen consumption rate in iPSC derived precursors on day 20 of differentiation and brown adipocytes after 40 days of replating in response to 1.5 μM oligomycin, 10 μM forskolin or vehicle control DMSO and 1 μM Rotenone and Antimycin (Rot/AA), respectively. Adipocytes or precursor cells were in XFe96 cell culture plates one week prior to the assay. Assay medium was prepared using Seahorse XF DMEM supplemented with 1 mM pyruvate, 2 mM glutamine and 10 mM glucose. Oxygen consumption rate was normalized by number of cells per well. Each data point represents Mean±SD, n=3, t-test, p=0.015.

FIGS. 9A-F: FIG. 9A—Schematic representation of cell isolation strategy for single cell RNA sequencing. Dorsal interscapular region was dissected out with intact dermis and epidermis for embryonic day (E) 13.5 and 14.5. For E15.5, skin and underlying dermis was removed before cell dissociation. For each stage, tissues were isolated from two embryos. Tissues were digested with enzymes followed by encapsulation using inDrops platform. Bar chart showing number of cells isolated from each stage from developing mouse embryos. The number represents sum of two replicates from each time point. E=embryonic day. FIG. 9B—UMAP embedding of the single cells isolated from E11.5, E12.5, E13.5, E14.5 and E15.5 mouse embryos 50 PC dimensions, 28244 cells). FIG. 9C—UMAP embedding showing cell clusters identified using Leiden based clustering on E11.5, E12.5, E13.5, E14.5, E15.5 mouse embryonic single cell data. FIG. 9D—Normalized confusion matrix showing contribution of cells from different time point to identified clusters. FIG. 9E—UMAP embedding showing expression of a curated list of cluster-specific genes. Scale represents log-normalized transcript counts. FIG. 9F—Dotplot showing expression of a curated list of cluster-specific genes. Scale represents log-normalized transcript counts.

FIGS. 10A-C: FIG. 10A—UMAP embedding showing expression of a curated list of cluster-specific genes. UMAP plots are colored by log-normalized transcript counts. FIG. 10B—Graph showing the probability mass flows from the progenitor cluster to the other indicated clusters as time increases based on waddington-OT transition matrix. FIG. 10C—Graph showing how probability mass flows from the brown adipocytes precursors cluster to brown adipocyte cluster as time increases based on waddington-OT transition matrix.

FIGS. 11A-G: FIG. 11A—UMAP plot generated from mouse single cell transcriptomics data from the developing perivascular adipose tissue on embryonic day 18 described in Angueira et al. 2021. Cells were clustered using Leiden clustering. FIG. 11B—UMAP plot generated from mouse single cell transcriptomics data from the developing mouse perivascular adipose tissue on embryonic day 18 described in Angueira et al. 2021. Cells were clustered using Leiden clustering and cell clusters representing the adipocyte lineage were selected. FIG. 11C—UMAP plot showing gene expression patterns in adipogenic cells sub-setted from the single cell transcriptomics data from the developing perivascular adipose tissue on embryonic day 18 described in Angueira et al. 2021. Scale represents log-normalized transcript counts. FIG. 11D—Heatmap showing gene expression patterns in adipogenic cells sub-setted from the single cell transcriptomics data from the developing perivascular adipose tissue on embryonic day 18 described in Angueira et al. 2021 and mouse datset generated in this study. Scale represents log-normalized transcript counts. FIG. 11E—Classifier based machine-learning classification of perivascular adipose cells. A kNN-classifier trained on mouse single cell clusters was used to predict identities of cell clusters described in Angueira et al. 2021 study. Heatmaps depict fraction of mouse cluster assignments for adipocyte, preadipocytes and progenitors identified in the perivascular adipose tissue. BA=brown adipocytes and BApre=brown adipocyte precursors identified in this study. FIG. 11F—Transverse section at the forelimb level of a E15.5 embryo stained for Dpp4 and Keratin 14 (Krt14) antibody to detect cells from BApre cluster. NT=Neural tube, n≥4. FIG. 11G—Transverse section from the interscapular region of a E15.5 embryo showing coexpression of Ly6a/Sca1 and Cd34 using antibody staining. NT=Neural tube, n≥4.

FIGS. 12A-C: FIG. 12A—Transverse section of Gata6-CreERT2:Rosa26-tdTomato E15.5 embryo at the forelimb level showing contribution of Gata6-Cre tdTomato positive cells in brown fat and muscle area, n≥3. FIG. 12B—Two representative images of RFP positive Gata6-tdTomato and Myh11 positive smooth muscle cells in E15.5 embryo at the forelimb level. Inset shows magnified image, n≥3. FIG. 12C—Transverse section of Gata6-CreERT2:Rosa26-tdTomato embryo at the forelimb level showing tdTomato and Dpp4 double positive cells at E13.5 and E15.5 n≥3.

FIGS. 13A-D: FIG. 13A—Flow cytometry analysis of day 2 of differentiating iPSC cultures to measure fraction of Mesogenin-Venus positive cells. Mean±SD, n=4. FIG. 13B—Schematic illustrating gene targeting strategy to generate PAX3-Venus knock-in iPSC line. FIG. 13C—Representative flow cytometry plots showing gating strategy for quantifying PAX3-Venus positive cells on day 8 of differentiation. FIG. 13D—UMAP embedding showing expression of a curated list of cluster-specific genes in human culture at day 20.

FIGS. 14A-C: FIG. 14A—Flow cytometry analysis of day 2 of differentiating iPSC cultures to measure fraction of Mesogenin-Venus positive cells. Mean±SD, n=4. FIG. 14B—Schematic illustrating gene targeting strategy to generate PAX3-Venus knock-in iPSC line. FIG. 14C—Representative flow cytometry plots showing gating strategy for quantifying PAX3-Venus positive cells on day 8 of differentiation. FIG. 14D—UMAP embedding showing expression of a curated list of cluster-specific genes in human culture at day 20.

FIGS. 15A-C: FIG. 15A—UMAP embedding showing cell clusters identified using Leiden based clustering on human culture at day 40. Colors indicate identified cell cluster. FIG. 15B—UMAP embedding showing expression of a curated list of cluster-specific genes in human culture at day 40. FIG. 15C—Machine-learning classification of human in-vitro cultured cells and mouse embryo. A k-NN classifier trained on clusters of mouse clusters was used to predict identities of the human in-vitro cultured progenitors and brown adipocytes precursors.

FIGS. 16A-B: FIG. 16A—UMAP embedding showing expression of a curated list of cluster-specific genes in human culture at day 60. FIG. 16B—Projection of the cell identities obtained from clustering analyses of individual time points onto the UMAP of the dataset combining the three time points.

FIGS. 17A-C: FIG. 17A—Merged UMAP embedding of the single cells isolated from day 20, 40 and 60 of human culture and E11.5, E12.5, E13.5, E14.5, E15.5 mouse embryos. Colors indicate both human and mouse clusters. FIG. 17B—Merged UMAP embedding of the single cells isolated from day 20, 40 and 60 of human culture and E11.5, E12.5, E13.5, E14.5, E15.5 mouse embryos. Colors indicate mouse clusters (left) and human clusters (right). FIG. 17C—Merged UMAP embedding of the single cells isolated from day 20, 40 and 60 of human culture and E11.5, E12.5, E13.5, E14.5, E15.5 mouse embryos. Colors indicate mouse embryonic days (left) and human culture days (right).

FIG. 18: Matrix plot showing transcriptional overlap between brown adipose specific genes in iPSC derived brown adipocytes (iPSC-BA), human fetal brown adipose tissue (fBAT), human embryonic stem cell derived brown adipocytes in Zhang et al (H9-d50) and iPSC derived brown adipocytes in Carobbio et al (KOLF2-C1-d25).

FIGS. 19A-B: FIG. 19A shows a schematic of the cell culture and organoids method of the disclosure. FIG. 19B shows a description of the cell culture methods of the disclosure, as organized by step and by days.

FIG. 20: FIG. 20 shows 4 images of a four month brown adipose tissue (BAT) organoid that was cryosectioned and stained with phalloidin. Lipid droplets were labeled, and the image is shown in inverted color.

### DETAILED DESCRIPTION

Brown Adipocytes (BAs) represent a specialized type of mammalian adipocytes able to unlink nutrient catabolism and ATP generation to dissipate energy as heat. This mechanism is physiologically important in mammals, allowing non-shivering thermogenesis to regulate body temperature in response to cold exposure. BA activity in humans shows an inverse correlation with body mass index and percentage of total body fat. Increasing brown fat activity either with drug treatment or with cell therapy approaches are considered promising approaches for the treatment of metabolic syndrome and obesity. Thus, the generation of brown adipocytes from human pluripotent stem cell has a huge potential for cell therapy and drug discovery.

Provided herein are novel differentiation methods to obtain functional BAs from human pluripotent stem cells. The methods include a treatment step with the signaling cues identified using FGF2, BMP7, TGFb1, a Wnt inhibitor and Triiodothyronin following dissociation and replating of the cultures to generate GATA6-positive adipocyte precursors. These progenitors can then be very efficiently differentiated into BA expressing the characteristic marker UCP1 after culture in adipogenic differentiation medium. The BAs differentiated under these conditions are functional, responding to adrenergic stress by lipolysis, heat production, and increased Oxygen consumption as compared to normal BAs. This protocol leads to the production of 2-dimensional cultures highly enriched in BA and BA precursors that could be used for drug screening or cell therapy.

### Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, the term “about” or “approximately” in the context of a numerical value or range means ±10% of the numerical value or range recited or claimed, unless the context requires a more limited range.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

By “reference” is meant a standard or control condition.

The terms “subject,” “patient,” “individual,” and the like as used herein are not intended to be limiting and can be generally interchanged. The subject is a mammal in need of such treatment, e.g., a human with a metabolic disorder.

As used herein, a “symptom” associated with a disorder includes any clinical or laboratory manifestation associated with the disorder, and is not limited to what the subject can feel or observe.

As used herein, “treating” encompasses, e.g., inhibition, regression, or stasis of the progression of a disorder. Treating also encompasses the amelioration of a symptom or symptoms of the disorder. As used herein, “inhibition” of disease progression or a disease complication in a subject means preventing or reducing the rate, frequency, or risk of disease progression and/or disease complications in the subject. The terms “preventing” and “prevention” refer to the administration of a therapeutic protocol to a clinically asymptomatic individual who is susceptible or predisposed to a particular adverse condition, disorder, or disease, and thus relates to reducing the risk of the occurrence of symptoms and/or their underlying cause.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Methods of Generating Gata6-Positive Brown Adipose Progenitor Cells

The present disclosure provides methods for generating BAT cells, preferably human BAT cells, from progenitor cells, e.g., paraxial mesoderm (presomitic mesoderm) (PAM) cells. PAM cells exhibit characteristics of progenitor cells of the Paraxial Mesoderm. In one embodiment, the PAM cells are characterized by the following properties:
a) they express one, two, or all three of the biomarkers Pax3, Myf5, and optionally Pax7, which are characteristic of Paraxial mesoderm progenitor cells, and
b) they are multipotent cells, capable of differentiating into at least brown adipose, endothelial, skeletal, dermis and muscle cell lineages in vivo and/or in vitro with the appropriate culture conditions

Optionally, they may have long term self-renewal properties, e.g., they can be maintained in culture more than 6 months. The multipotency of the PAM cells can be tested in vitro, e.g., by in vitro differentiation into adipose, skeletal, dermal or muscle cell lineages using the protocols described below, and in particular in the Examples.

In some embodiments, the progenitor cells, e.g., paraxial mesoderm cells, are induced paraxial mesoderm (iPAM) cells, preferably derived from induced pluripotent stem (iPS) cells or embryonic stem (ES) cells, e.g., human pluripotent stem cells. Methods for obtaining and generating PAM cells are known in the art; see, e.g., WO2013030243 and Chal et al. 2015; Loh et al., Cell. 2016 Jul. 14; 166(2):451-67; Shelton et al., Stem Cell Reports. 2014 Sep. 9; 3(3): 516-529; Xi et al., Cell Rep. 2017 Feb. 7; 18(6): 1573-1585; Choi et al., Cell Rep. 2016 Jun. 7; 15(10):2301-12; and Hosoyama et al., Stem Cells Transl Med. 2014 May; 3(5):564-74.

In some embodiments, the methods include culturing iPS or ES cells in the presence of one a Wnt activator, e.g., a Wnt ligand, GSK3beta inhibitor, or member of the R-spondin family, or of both a Wnt activator and a BMP inhibitor, e.g., an ALK inhibitor. In some embodiments, progenitor cells isolated from a subject, e.g., autologous or primary cells, are differentiated using the described media; methods for isolating CD34+ BAT progenitor cells from the skeletal muscle of a subject are described in US PG Pub 20160303100.

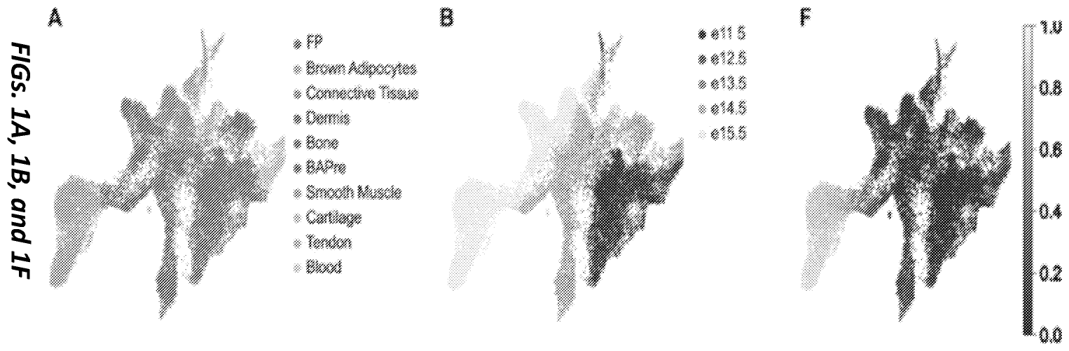

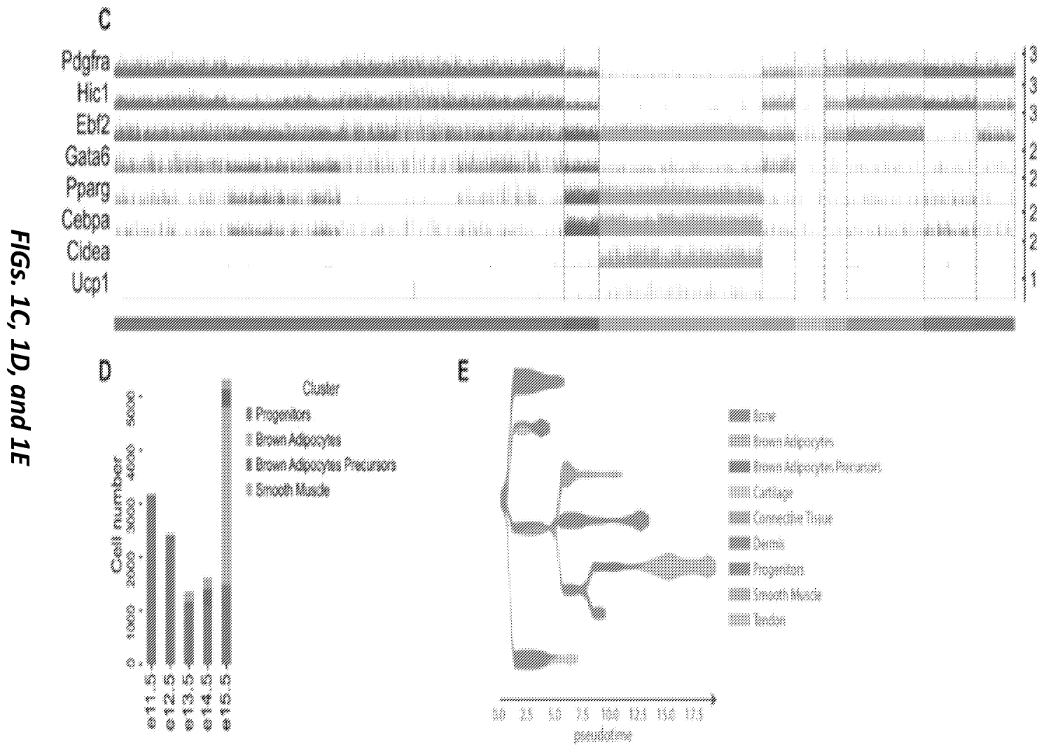

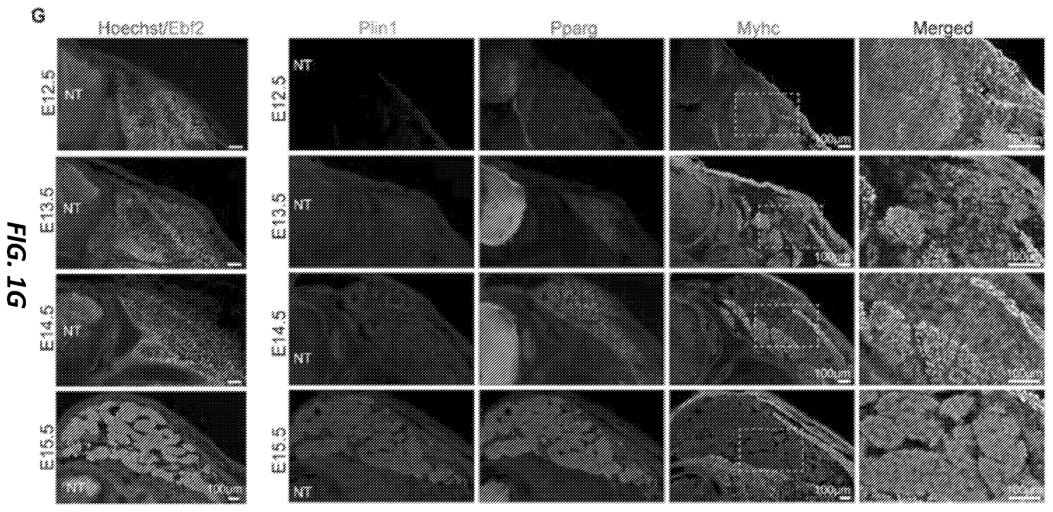

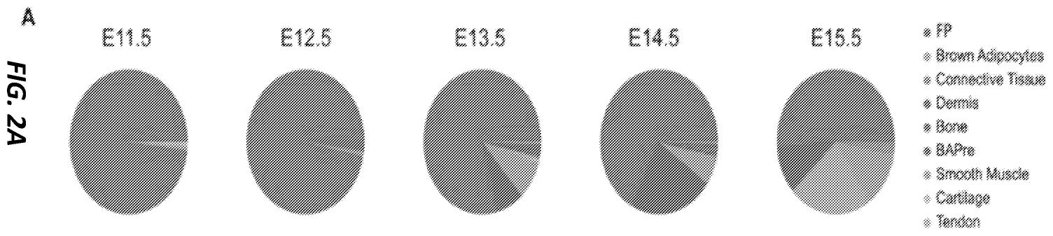

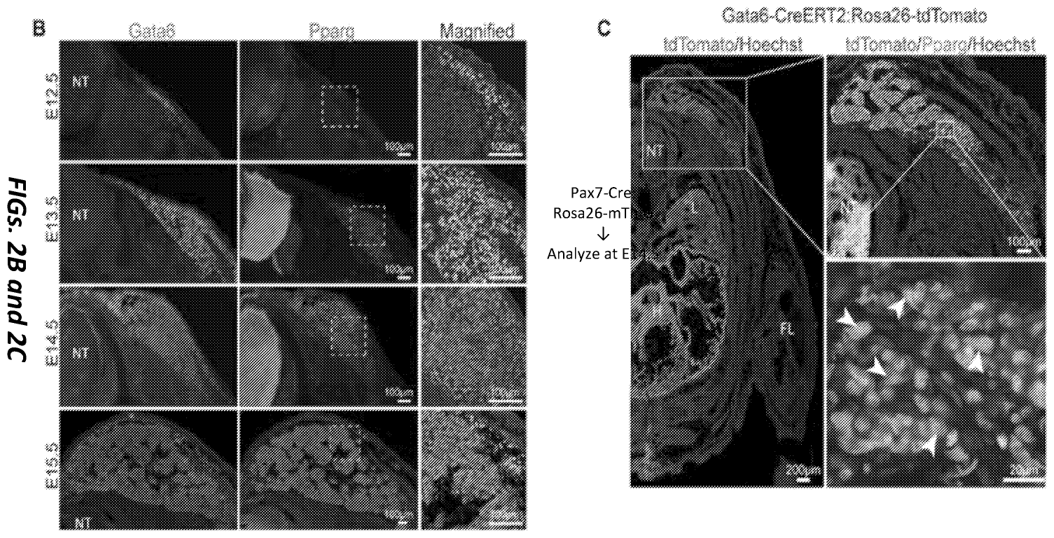

Studying brown fat lineage development has proven complicated due to the difficulty of identifying intermediate stages in differentiation. Single cell sequencing techniques together with lineage tracing methods now provide powerful approaches for exploring cell states during differentiation programs. Disclosed herein is a characterization of the development of interscapular mouse brown fat using scRNAseq. The development of paraxial mesoderm-derived cell populations in the dorsal trunk at the forelimb level of mouse embryos which contains the interscapular brown fat was analyzed. This analysis identified a previously unrecognized brown adipocyte differentiation stage characterized by expression of the transcription factor Gata6. Using bioinformatics tools and lineage tracing analysis in mouse, reveal that these cells derive from a multipotent fibroblastic precursor population and give rise to Pparg-positive preadipocytes which differentiate into UCP1-positive brown adipocytes (FIGS. 1E, 2B).

Our data also suggest that signaling cues known to be important for brown adipogenesis such as Wnt inhibition and BMP activation act at the Gata6-positive precursor stage. We show that Bmp4/7 and Bmpr1a/2 are enriched in the Gata6-positive progenitors. This is consistent with the well-established role of Bmps in the promotion of brown adipogenesis (Schulz et al., 2013; Tseng et al., 2008). We also observed transient expression of the Wnt inhibitors Sfrp1, 2 and 4 and Dkk2 in the Gata6-positive precursors, consistent with the well-known anti-adipogenic role of Wnt signaling (Bagchi and MacDougald, 2021; Longo et al., 2004). The thyroid hormone and the FGF/IGF1/PI3K pathways which play a role in the control of thermogenesis and brown adipose development and physiology were also activated in the Gata6 precursors (Mullur et al., 2014; Ohta and Itoh, 2014). We modified a protocol to produce highly efficient generation of functional human brown adipocytes in vitro using defined serum-free culture conditions. ScRNAseq analysis of the human cells differentiating in vitro show that they recapitulate a developmental sequence very similar to that observed in mouse in vivo. This sequence results in the production of GATA6-positive brown adipocyte precursors and then to PPARG-positive preadipocytes and finally to UCP1-positive adipocytes which exhibit functional properties characteristic of brown adipocytes.

Our analyses using the STREAM or Waddington OT pipelines suggest that brown adipocyte precursors share a common precursor with other somite-derived mesenchymal lineages such as dermis, muscle connective tissue, cartilage precursors and smooth muscle (FIG. 1E) (Chen et al., 2019; Schiebinger et al., 2019). In contrast, the skeletal muscle cluster is clearly segregated from the mesenchymal cluster in our dataset. This is consistent with the early divergence of the Pax7-expressing multipotent progenitors into skeletal muscle lineage by E11.5 revealed by lineage tracing experiments in mouse (Lepper and Fan, 2010). Lineage tracing analyses in mouse demonstrated that interscapular brown fat derives from Pax3/Myf5+ and Pax3/Pax7/Myf5+ cells (Sebo et al., 2018). Our lineage tracing experiments confirm that some of the mouse interscapular brown fat derives from Pax7-expressing precursors (Lepper and Fan, 2010; Sanchez-Gurmaches and Guertin, 2014) which can give rise to the Gata6-positive brown adipocyte precursor population. These early Pax7 precursors could correspond to the En1-positive cells of the central dermomyotome previously identified in mouse (Atit et al., 2006). We reanalyzed a scRNAseq dataset of developing mouse periaortic BAT and observed a population of Gata6-positive BA precursors closely related to the one identified in the interscapular BAT (Angueira et al., 2021). Interestingly, periaortic BAT receives little contribution from Pax3+ cells and has no contribution from Myf5+ cells (Sanchez-Gurmaches and Guertin, 2014). This therefore suggests that different embryonic populations can contribute to the Gata6-positive brown adipocyte precursors.

Gata6 (GATA binding protein 6), is a zinc finger transcription factor which plays an essential role in the development of extraembryonic tissues (Koutsourakis et al., 1999). Its null mutation in mouse leads to lethality shortly after implantation. Gata6 has also been implicated in the development of several organs including heart, lung, gut, pancreas, and skin but its expression during brown fat development has so far not been reported (Freyer et al., 2015; Morrisey et al., 1996). Gata6 was however identified in silico as a transcription factor potentially regulating the expression of brown fat specific genes such as Zic1 and TCA cycle enzymes such as citrate synthase (Cs) in the ProFat database (Cheng et al., 2018). Also, a GATA recognition motif was identified in ATACseq data of differentiating human adipocytes in vitro (Zhang et al., 2020). In cardiomyocytes, GATA-6 was shown to associate with Ppara to regulate the expression of the glucose transporter Glut4 (Yao et al., 2012). Together, these data suggest that Gata6 could play a role in the regulation of energy metabolism in differentiating brown adipocytes.

Our transcriptome analysis of the brown adipocyte lineage development in mouse identified signaling cues which allowed us to modify our paraxial mesoderm development protocol to generate brown fat from hPSCs. This also provided us with a benchmark to which the differentiating hPSCs could be compared. Our comparison of the differentiated human adipocytes generated in vitro in our protocol with those generated by two other recently published protocols (Carobbio et al., 2021; Zhang et al., 2020) suggest that the transcriptional signature of the adipocytes generated in our conditions is closer to endogenous fetal brown adipocytes. In humans, BAT activity shows an inverse correlation with body mass index and percentage of total body fat (Cypess et al., 2009; Saito et al., 2009). Moreover, the graft of ectopic BAT leads to improvement of glucose metabolism in diabetic or obese mice (Gunawardana and Piston, 2012; Stanford et al., 2013). Our work could therefore help the development of cell therapy strategies involving the graft of human brown adipocytes generated in vitro for the treatment of obesity, metabolic syndrome, and diabetes.

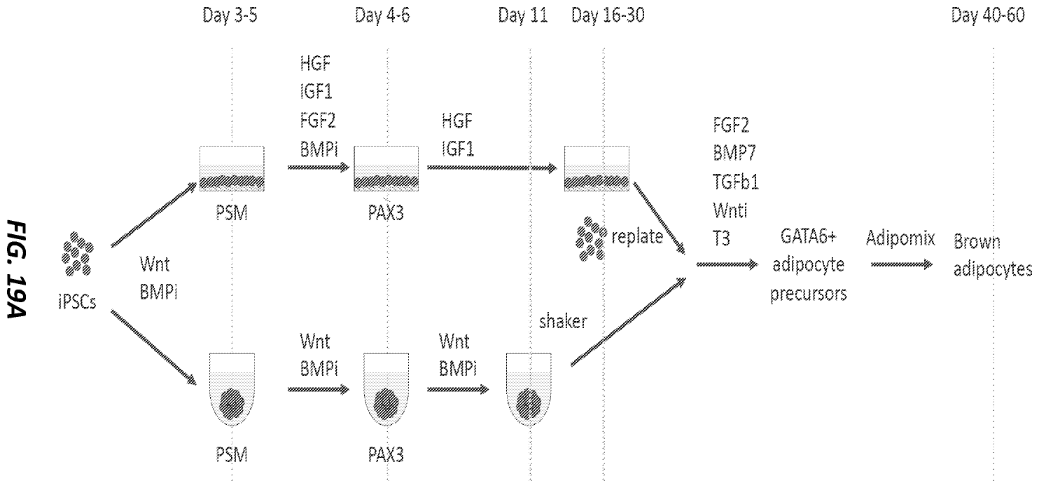

Methods for generating BAT and/or BA progenitor cells from source cells, e.g., embryonic stem cells (ESC) or induced pluripotent stem cells (iPSCs), as described herein can include an induction step of culturing PAX3-positive progenitors in the presence of a cocktail comprising FGF2/BMP7/TGBb1/a Wnt inhibitor/T3, preferably in adherent conditions or organoid conditions; an example is shown in FIG. 19A. The BA progenitor cells and/or BAT are particularly suited for cell therapy applications.

### Adherent Conditions

In some embodiments, Gata6-positive cells are produced by the following methods. In some embodiments, human induced pluripotent stem cells (iPSCs) are dissociated to single cells using enzymes and seeded at a density of 10,000-50,000/cm² on cell-culture dishes in feeder-free maintenance medium supplemented with a ROCK inhibitor. In some embodiments, the cells make small pluripotent colonies and are treated with basal medium supplemented with a basal medium which includes insulin-transferrin-selenium, a GSK3 inhibitor, and a BMP inhibitor (presomitic mesoderm/PSM medium) for between 2 and 5 days. In some embodiments, over the next 3 days, the cells are cultured in a basal medium supplemented with basal medium which includes insulin-transferrin-selenium, a GSK3 inhibitor, and a BMP inhibitor, and an FGF activator. To differentiate cells into PAX3-positive somitic progenitors, in some embodiments, cells are treated with high glucose media supplemented with serum free media, non-essential amino acid supplement, synthetic basal medium, an HGF activator, an IGF activator, an FGF activator, and a BMP inhibitor for between 1-5 days. In some embodiments, the cells were further treated with a Wnt activator to differentiate into PAX3-positive somitie progenitors. In some embodiments, the PAX3-positive cells are further differentiated into PAX7 and EBF2-positive progenitors in high glucose media supplemented with serum free media, non-essential amino acid supplement, synthetic basal medium, an HGF activator, an IGF activator, for between 5-30 days. To generate brown adipocyte precursors, in some embodiments, the cells are dissociated to single cells enzymes in PBS, filtered, and seeded at a density of 30,000-150,000/cm² on cell-culture dishes and treated with high glucose media supplemented with serum-free media, a basal medium which includes insulin-transferrin-selenium, an FGF activator, PROCN inhibitor (Wnt inhibitor), a BMP activator, a TGFβ activator, and a thyroid hormone receptor activator (together referred to as adipocyte precursor medium) for between 1-10 days. In some embodiments, after 4-6 days cells are cultured in high glucose media supplemented with serum-free medium, a basal medium which includes insulin-transferrin-selenium, a phosphodiesterase inhibitor, an antioxidant (e.g., vitamin C), a thyroid hormone receptor activator, a TGFβ inhibitor, corticosteroid, an EGF activator, a second corticosteroid, and insulin sensitizer (together referred to as adipogenic differentiation medium) for between 20-50 days, wherein the adipogenic differentiation medium is changed every 2-3 days.

In some embodiments, human induced plutipotent stem cells (iPSCs) are dissociated to single cells using Accutase and seeded at a density of 30,000-33,000/cm² on Matrigel-coated dishes in mTESR-1 supplemented with 10 μM Y-27632. In some embodiments, the cells make small pluripotent colonies and are treated with DMEM/F12 GlutaMAX supplemented with 1% ITS, 3 μM CHIR99021, and 0.5 μM LDN-193189 (PSM medium) for 3 days. In some embodiments, over the next 3 days, the cells are cultured in DMEM/F12 GlutaMAX supplemented with 1% ITS, 3 μM CHIR99021, 0.5 μM LDN-193189, and 20 ng/ml FGF-2. To differentiate cells in PAX3-positive somitic progenitors, in some embodiments, cells are treated with Dulbecco's Modified Eagle Medium (DMEM) high glucose media supplemented with 15% KSR, 1× Non-Essential Amino Acid (NEAA) Solution, 0.01 mM bME, 10 ng/ml HGF, 2 ng/ml IGF-1, 20 ng/ml FGF-2, and 0.1 μM LDN-193189 for 2 days. In some embodiments, the PAX3-positive cells are further differentiated into PAX7 and EBF2-positive progenitors in DMEM high glucose supplemented with 15% KSR, 1×NEAA, 0.01 mM bME, 10 ng/ml HGF, and 2 ng/ml IGF-1 for between 8-23 days. To generate brown adipocyte precursors, in some embodiments, the cells are dissociated to single cells using Type IV Collagenase and Trypsine EDTA in PBS, filtered through a 30 μm filter and seeded at a density of 60,000-100,000/cm² on Matrigel-coated dishes and treated with DMEM high glucose supplemented with 5% KSR, 1% ITS, 2 ng/ml FGF-2, 20 nM C59, 20 ng/ml BMP7, 10 ng/ml TGFb1, 2 nM T3 (Adipocyte precursor medium) for between 4-6 days. In some embodiments, after 4-6 days cells are cultured in DMEM high glucose supplemented with 5% KSR, 1% ITS, 500 μM IBMX, 25.5 g/ml L-Ascorbic acid, 2 nM T3, 5 μM TGFb inhibitor SB431542, 1 μM Dexamethasone, 10 ng/ml EGF, 4 μg/ml Hydrocortisone, 1 μM Rosiglitazone (adipogenic differentiation medium) for between 30-40 days, wherein the adipogenic differentiation medium is changed every 2-3 days.

Recombinant proteins, activators/agonists, and antagonists/inhibitors are typically supplemented each at 0.1 ng/mL to 1 mg/mL of culture medium. Small compounds/molecules activators or inhibitors are typically supplemented each at 1 nM to 1 mM. Then, cultures are changed to an adipocyte differentiation medium, and refreshed every 2-3 days. Adipogenic differentiation medium comprises a DMEM-based medium containing a serum replacement supplement as known in the art or described herein, 1× Insulin-Transferin-Selenium (ITS, Gibco) and further comprising 0.5 mM isobutylmethylxanthine (IBMX), 125 nM indomethacin, 1 nM triiodothreonine (T3), 5 mM dexamethasone, and 1 mM rosiglitazone (see also a related adipogenic cocktail described in Sharma et al, PO 2014 and US20150030662).

### Organoid/Somitoid Conditions

Provided herein are methods to produce BAs and/or BAT in 3D. In some embodiments, BAs containing organoids can be obtained from source cells, e.g., ESCs or iPSCs, e.g., human iPSCs, by a protocol that involves an induction step of culturing PAX3-positive progenitors with FGF2/BMP7/TGBb1/a Wnt inhibitor/T3 treatment described in the protocol detailed above. Briefly, in some embodiments, iPSC cultures are dissociated to single cells using enzymes and aggregated in non-adhesive plates to form 3D structures. These structures, called somitoids, are cultured in basal medium supplemented with a basal medium which includes insulin-transferrin-selenium, a GSK3 inhibitor, and a BMP inhibitor (presomitic mesoderm/PSM medium) supplemented with serum to generate PAX3-expressing somite-like structures and dermomyotomal cells. In some embodiments, the cells were further treated with a Wnt activator to generate PAX3-expressing somite-like structures. In some embodiments, somitoids are then cultured in high glucose media supplemented with with serum-free media, a basal medium which includes insulin-transferrin-selenium, an FGF activator, PROCN inhibitor (Wnt inhibitor), a BMP activator, a TGFβ activator, and a thyroid hormone receptor activator (together referred to as adipocyte precursor medium) for between 2-15 days. In some embodiments, to differentiate the Gata6-positive precursors into lipid vesicle containing BA, somitoids are cultured in the adipogenic differentiation medium (high glucose media supplemented with serum-free medium, a basal medium which includes insulin-transferrin-selenium, a phosphodiesterase inhibitor, an antioxidant (e.g., vitamin C), a thyroid hormone receptor activator, a TGFβ inhibitor, corticosteroid, an EGF activator, a second corticosteroid, and insulin sensitizer (together referred to as adipogenic differentiation medium) for between 20-40 days. This method leads to the production of tridimensional organoids highly enriched in UCP1 positive BAs. These organoids act as glucose sinks, and serve as a substrate to graft in patients for cell therapy of obesity, type II diabetes, or metabolic syndrome.

Provided herein are methods to produce BAs and/or BAT in 3D. In some embodiments, BAs containing organoids can be obtained from source cells, e.g., ESCs or iPSCs, e.g., human iPSCs, by a protocol that involves an induction step of culturing GATA6+BA progenitors with FGF2/BMP7/TGBb1/T3 treatment described in the protocol detailed above. Briefly, in some embodiments, iPSC cultures are dissociated to single cells using Accutase and aggregated in non-adhesive U-bottom plates to form 3D structures. These structures, called somitoids, are cultured in the DMEM/F12 GlutaMAX supplemented with 1% ITS, 3 μM CHIR99021, and 0.5 μM LDN-193189 (PSM medium) supplemented with 5% FCS for 11 days to generate PAX3-expressing somite-like structures and dermomyotomal cells. In some embodiments, somitoids are then cultured in DMEM high glucose supplemented with 5% KSR, 1% ITS, 2 ng/ml FGF-2, 20 nM C59, 20 ng/ml BMP4, 10 ng/ml TGFb1, 2 nM T3 (Adipocyte precursor medium) for between 4-12 days. In some embodiments, to differentiate the precursors into lipid vesicle containing BA, somitoids are cultured in the adipogenic differentiation medium (DMEM high glucose supplemented with 5% KSR, 1% ITS, 500 μM IBMX, 25.5 g/ml L-Ascorbic acid, 2 nM T3, 5 μM TGFb inhibitor SB431542, 1 μM Dexamethasone, 10 ng/ml EGF, 4 g/ml Hydrocortisone, 1 μM Rosiglitazone) for between 20-40 days. This method leads to the production of tridimensional organoids highly enriched in UCP1 positive BAs. These organoids act as glucose sinks, and serve as a substrate to graft in patients for cell therapy of obesity, type II diabetes, or metabolic syndrome.

Recombinant proteins, activators/agonists, and antagonists/inhibitors are typically supplemented each at 0.1 ng/mL to 1 mg/mL of culture medium. Small compounds/molecules activators or inhibitors are typically supplemented each at 1 nM to 1 mM. Then, cultures are changed to an adipocyte differentiation medium, and refreshed every 2-3 days. Adipogenic differentiation medium comprises a DMEM-based medium containing a serum replacement supplement as known in the art or described herein, 1× Insulin-Transferin-Selenium (ITS, Gibco) and further comprising 0.5 mM isobutylmethylxanthine (IBMX), 125 nM indomethacin, 1 nM triiodothreonine (T3), 5 mM dexamethasone, and 1 mM rosiglitazone (see also a related adipogenic cocktail described in Sharma et al, PO 2014 and US20150030662).

### Wnt Activators and Inhibitors

The present methods can include the use of agonists or activators of the canonical Wnt/beta catenin signaling pathway, characterized by a Wnt dependant inhibition of glycogen synthase kinase 3β (GSK-3β), leading to a subsequent stabilization of β-catenin, which then translocates to the nucleus to act as a transcription factor. As used herein the term “activator” denotes a molecule, e.g., antibody, protein, nucleic acid, or small molecule that enhances Wnt signaling activity. For example, for the canonical Wnt/β-catenin signaling pathway, this activity can be measured by Wnt reporter activity using established multimers of LEF/TCF binding sites reporters, and/or inhibition of GSK-3β, and/or activation of canonical Wnt target genes such as T, Tbx6, Msgn1, or Axin2.

In some embodiments, GSK-3β inhibitors activate the Wnt pathway. Inhibitors of GSK-3β are known in the art and include lithium chloride (LiCl), Purvalanol A, olomoucine, alsterpaullone, kenpaullone, SB216763 (3-(2,4-dichlorophenyl)-4-(1-methyl-1H-indol-3-yl)-1H-pyrrole-2,5-dione) and SB415286 (3-[(3-chloro-4-hydroxyphenyl)amino]-4-(2-nitrophenyl)-1H-pyrrole-2,5-dio-ne) which are maleimide derivatives, benzyl-2-methyl-1,2,4-thiadiazolidine-3,5-dione (TDZD-8), 2-thio(3-iodobenzyl)-5-(1-pyridyl)-[1,3,4]-oxadiazole (GSK3 inhibitor II), 2,4-dibenzyl-5-oxothiadiazolidine-3-thione (OTDZT), (2′Z,3′E)-6-Bromoindirubin-3′-oxime (BIO), a 4 Dibromoacetophenone (i.e., Tau Protein Kinase I (TPK I) Inhibitor), 2-Chloro-1-(4,5-dibromo-thiophen-2-yl)-ethanone, N-(4-Methoxybenzyl)-N′-(5-nitro-1,3-thiazol-2-yl)urea (AR-A014418), and indirubins (e.g., indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5, 5′-dibromoindirubin; 5-nitroindirubin; 5-chloroindirubin; 5-methylindirubin, 5 bromoindirubin), 4-Benzyl-2-methyl-1,2,4-thiadiazolidine-3,5-dione (TDZD-8), 2-thio(3-iodobenzyl)-5-(1-pyridyl)-[1,3,4]-oxadiazole (GSK3 inhibitor II), 2,4-Dibenzyl-5-oxothiadiazolidine-3-thione (OTDZT), (2′Z,3′E)-6-Bromoindirubin-3′-oxime (BIO), a 4 Dibromoacetophenone (i.e., Tau Protein Kinase I (TPK I) Inhibitor), 2-Chloro-1-(4,5-dibromo-thiophen-2-yl)-ethanone, (vi) N-(4-Methoxybenzyl)-N′-(5-nitro-1,3-thiazol-2-yl)urea (AR-A014418), and H-KEAPPAPPQSpP-NH2 (L803) or its cell-permeable derivative Myr-N-GKEAPPAPPQSpP-NH2 (L803-mts). Other GSK30 inhibitors are disclosed in U.S. Pat. Nos. 6,417,185; 6,489,344; 6,608,063 and Published U.S. Applications Nos. 20160375006 2; 0040138273; 20040106574; 20040077707; 20040034037; 20030216574; and 20030130289.

Other activators of Wnt signaling include WAY-316606 (SFRP Inhibitor) Bodine et al., Bone. 2009 June; 44(6):1063-8; (hetero)arylpyrimidines (Gilbert et al., Bioorg Med Chem Lett. 2010 Jan. 1; 20(1):366-70); IQ1 (PP2A Activator) Miyabayashi et al., Proc Natl Acad Sci USA. 2007 Mar. 27; 104(13):5668-73 (2007); QS11 (ARFGAP1 Activator) Zhang et al., Proc Natl Acad Sci USA. 2007 May 1; 104(18):7444-8; SB-216763 (GSK3 Inhibitor) Coghlan et al., Chem Biol. 2000 October; 7(10):793-803 (2000); CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile, GSK3 Inhibitor); DCA (beta-catenin Activator) Pai et al., Mol Biol Cell. 2004 May; 15(5):2156-63; and 2-amino-4-[3,4-(methylenedioxy) benzyl-amino]-6-(3-methoxyphenyl) pyrimidine (AMBMP) Liu et al., Inflamm Res. 2016 January; 65(1):61-9. Wnt activators can be effective in the methods provided herein at a range between 0.1 micromolar and 10 micromolar. For example, the Wnt activators described herein can be used at 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, 2.0 μM, 3 μM, 4, μM, 5 μM, 6 μM, 7 μM, 8 μM, 9 μM, 10 μM, and any concentrations in between.

Wnt ligands in natural or modified forms can also be used as Wnt activator in the form of purified or recombinant proteins. Wnt ligands comprise a large family of Wnt activators including but not limited to Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, Wnt-3. The R-spondins family encompassing R-spondin1, R-spondin2, R-spondin3, and R-spondin4 are potent Wnt signaling enhancer through their binding to LGR4/5 surface receptors. Natural or modified, synthetic, polypeptides fragments generated from known Wnt activator polypeptide sequence can also be potential Wnt activators. When the activator of the Wnt signaling pathway is a protein, it may be a purified protein or a recombinant protein or a synthetic protein. Alternatively, conditioned media produced from a cell line engineered to express one or several Wnt ligands can be used as a Wnt activator. Wnt signaling can also be activated by blocking negative regulators of Wnt signaling, such as Axin and APC using RNA interference.

In some embodiments, the methods disclosed herein use Wnt inhibitors. The terms “Wnt antagonist”, “Wnt inhibitor”, and “inhibitor of Wnt signaling” are used interchangeably herein to mean an agent that antagonizes, inhibits, suppresses, or negatively regulates Wnt modulation of a cell's activity. Likewise, the phrases “antagonizing Wnt signaling” and “inhibiting Wnt signaling” are used interchangeably herein to mean antagonizing, inhibiting, or otherwise negatively regulating Wnt modulation of a cell's activity. Wnt inhibitors may act anywhere along a Wnt signaling pathway to antagonize Wnt signaling. For example, Wnt inhibitors may antagonize activation of the Wnt co-receptors, for example by blocking Wnt binding; or they may antagonize the activity of a protein in a Wnt-responsive intracellular signaling cascade, e.g., the Wnt/β-catenin, Wnt/PCP or Wnt/Ca2+ signaling pathways, for example by promoting β-catenin degradation or preventing translocation of stabilized β-catenin to the nucleus. Wnt inhibitors can include small-molecule inhibitors and small proteins.

Non-limiting examples of Wnt inhibitors include members of the Dkk family (Dickkopf proteins), including Dkk-1, Dkk-2, Dkk-3, Dkk-4, Soggy-1/DkkL1. Further examples of Wnt inhibitors include secreted frizzled related proteins (sFRPs) including sFRP-1, sFRP-, sFRP-3, sFRP-4, and sFRP-5. Further examples of Wnt inhibitors include can be a porcupine inhibitor. Other examples of Wnt inhibitors include APCDD1, APCDD1L, Draxin, IWP, IWP-2, IWP-4, Ginsenoside Rh4, M2912, KY-05009, ICG-001; INCB28060/INC280/NVP-INC280, IWR-1-endo, KY02111, FH535, WIKI4, CCT251545, Salinomycin, Prodigiosin, IQ-1, KYA1797K, NCB-0846, PNU-74654, LF3, iCRT14, Adavivint, PRI-724, Triptonide, M435-1279, Resibufogenin, LGK974, C59, ETC-159, JW55, RCM-1, MSAB, Ant1.4Br/Ant 1.4C1, Niclosamide, apicularen, bafilomycin, XAV939, IWR, G007-LK, G244-LM, Lanatoside C, pyrvinium, NSC668036, 2,4-diamino-quinazoline, Quercetin, Isoquercitrin, ICG-001, PKF115-584, BC2059/Tegavivint, Shizokaol D, IGFBP-4, LMBR1L, Notum, SOST/Sclerostin, USAG1, WIF-1. Wnt inhibitors can be effective in the methods provided herein at a range between 0.1 micromolar and 10 micromolar. For example, the Wnt inhibitors described herein can be used at 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, 2.0 μM, 3 μM, 4, μM, 5 μM, 6 μM, 7 μM, 8 μM, 9 μM, 10 μM, and any concentrations in between.

### BMP Activators and Inhibitors

A bone morphogenetic protein (BMP) activator or agonist refers to a molecule. e.g., antibodies, protein, nucleic acids, or small molecules that enhances BMP signaling activity. Typically, a BMP signaling pathway agonist/activator binds to or directly activates components of the biological pathway in which BMP participates, such as a BMP receptor protein (e.g. BMP type I receptors ALK2 and/or ALK3) or downstream SMAD proteins). Examples of known BMP signaling activators include BMP7, BMP4, and BMP2. BMP activators can be effective in the methods provided herein at a range between 1 ng/ml and 1000 ng/ml. For example, the BMP activators described herein can be used at 1 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, and any concentrations in between. A bone morphogenetic protein (BMP) antagonist or inhibitor refers to a molecule, e.g., antibodies, proteins, nucleic acids, or small molecules, that inhibits or attenuates the biological activity of the BMP signaling pathway either by directly interacting with BMP or by acting on components of the biological pathway in which BMP participates, such as a BMP receptor protein (e.g. BMP type I receptors ALK2 and/or ALK3) or downstream SMAD proteins). Typically, a compound is deemed to be an inhibitor of the BMP signaling pathway if, after culturing cells in the presence of said compound, the level of phosphorylated Smad 1, 5 or 8 is decreased compared to cells cultured in the absence of said compound. Levels of phosphorylated Smad proteins can be measured by Western blot using antibodies specific for the phosphorylated form of said Smad proteins.

Examples include noggin, an inhibitor of the transduction activity of the BMP type I receptors ALK2 and/or ALK3, chordin, or LDN-193189 (DM3189), a dorsomorphin derivative. Noggin, chordin, follistatin, and gremlin block BMP signaling by sequestrating secreted BMP, preventing its binding to the receptor. The inhibitor of the BMP signaling pathway may be a BMP antagonist, a chemical compound that blocks BMP type I and/or type II receptors activity (BMP type I/II receptor inhibitor), an inhibitor of BMP type I and/or type II gene expression, or a molecule which inhibits any downstream step of the BMP signaling pathway. The inhibitor of BMP signaling may be a natural or a synthetic compound. When the inhibitor of the BMP signaling pathway is a protein, it may be a purified protein or a recombinant protein or a synthetic protein. Non-limiting examples of BMP inhibitors include A77-01, A83-01, LDN193189 dihydrochloride, dormorphin dihydrochloride, DMG-1, SB505124, ML347, M4K2163, K02288, DMH1, DMH2, dorsomorphin, noggin, follistatin, and cerberus.

It is well known in the art that an inhibitor of BMP type I receptors may block the BMP signaling pathway, see for example Yu et al, Nat Chem Biol. 2008.

In one non-limiting example, the inhibitor of BMP type I receptors is Dorsomorphin, a chemical compound or any derivatives thereof generated by structure-activity studies (Cuny G D et al., 2008), e.g., LDN-193189. Dorsomorphin (6-[4-(2-Piperidin-1-yl-ethoxy)phenyl]-3-pyridin-4-yl-pyrazolo[1,5-a]pyrimidine, also known as Compound C, specifically inhibits BMP type I receptors (ALK2, 3, and 6) (Yu P B et al., 2008). For additional BMP inhibitors, see WO2013030243, which is herein incorporated by reference in its entirety. BMP inhibitors can be effective in the methods provided herein at a range between 1 nM and 1000 nanomolar. For example, the BMP inhibitors described herein can be used at 1 nM, 10 nM, 100 nM, 200 nM, 300 nM, 400 nM, 500 nM, 600 nM, 700 nM, 800 nM, 900 nM, 1000 nM, and any concentrations in between.

### Hepatocyte Growth Factor (HGF) Activator Agonist

A Hepatocyte Growth factor signaling pathway agonist/activator is a molecule e.g., antibody, protein, nucleic acid, or small molecule, that enhances HGF signaling activity. Typically, a HGF signaling pathway agonist/activator binds to or directly activates the c-Met proto-oncogene (HGFR) receptor. Examples of known HGF signaling activators include Hepatocyte Growth Factor-Scatter Factor (HGF-SF), HGF variants such as described further in U.S. Pat. Nos. 5,227,158; 5,316,921 and 5,328,837; HGFR activating antibodies such as MAb DO-24, 6E10 or 3D6 described in Pietronave et al, Am J Physiol Heart Circ Physiol. 2010 April; 298(4):H1155-65 and U.S. Ser. No. 08/884,669. Among the intracellular signaling pathways transduced by HGFR activation are MAPK, STAT3, and PI3K/Akt signaling axis. These intracellular pathways can be monitored using biochemical reporter assays and for transduction cascade activation, as known in the art. HGF signaling activity can be measured by biological assays such as mitogenic, motogenic, or morphogenic activities as a result of HGF binding to a HGF receptor. In particular, c-MET activation can lead to the disruption of cadherin-based cell-cell contacts, and promote cell motility as evidenced by a cell-scattering phenotype, which was first described with MDCK cells treated with HGF (Zhu et al., Cell Growth Differ. 1994 April; 5(4):359-66). HGF activators can be effective in the methods provided herein at a range between 1 and 1000 ng/ml. For example, the HGF activators described herein can be used at 1 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, and any concentrations in between.

### Insulin-Like Growth Factor (IGF) Activator/Agonist

An Insulin-like Growth factor signaling pathway agonist/activator is a molecule e.g., antibody, protein, nucleic acid, or small molecule that enhances IGF signaling activity. Examples of known IGF activators include IGF-1 (e.g., somatomedin C), MGF, IGF-2 and Insulin, demethylasterriquinone B-1, (DMAQ-B1; Salituro et al, Recent Prog Horm Res. 2001; 56:107-26). Typically, an IGF signaling pathway agonist/activator binds to or directly activates IGF1 receptor (IGF1Ra, b), and/or the insulin receptor (IR), and/or Insulin receptor-related receptor IR-related receptor (IRR). IGF signaling transduction results in the activation of several intracellular pathways including RAS-MAP kinase pathway, PI3K/AKT, and PI3K/mTor signaling pathways. These intracellular pathways activity can be monitored using biochemical reporter assays and transduction cascade activation, as known in the art. IGF activators can be effective in the methods provided herein at a range between 1 and 1000 ng/ml. For example, the IGF activators described herein can be used at 1 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, and any concentrations in between.

### Fibroblast Growth Factor Activator/Agonist

A Fibroblast Growth factor signaling pathway agonist/activator is a molecule e.g., antibody, protein, nucleic acid, or small molecule that enhances FGF signaling activity. Examples of FGF signaling pathway agonist/activator are natural or recombinant proteins ligands including the 23 identified FGF ligands, e.g., FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, and FGF-23. FGF activators bind and/or activate one or several FGF receptors (FGFR1 to 4) leading to the activation of several signaling pathways including RAS-MAPK, PI3K-AKT, PLCγ, and STAT signaling pathways. These intracellular pathways activity can be monitored using biochemical reporter assays and transduction cascade activation, as known in the art. FGF activators can be effective in the methods provided herein at a range between 1 and 1000 ng/ml. For example, the FGF activators described herein can be used at 1 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, and any concentrations in between.

### TGFβ Activators and Inhibitors

A TGFβ signaling pathway activator is a molecule, e.g., an antibody, protein, nucleic acid, or small molecule that enhances TGFβ signaling activity. Typically, TGFβ agonist/activator binds to or activates the TGF-β receptor (TGF-βR). Examples of TGFβ activators include, but are not limited to, TGFβ1, TGFβ2, and TGFβ3. TGFβ activators can be effective in the methods provided herein at a range between 1 and 1000 ng/ml. For example, the TGFβ activators described herein can be used at 1 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, and any concentrations in between.

A TGFβ signaling pathway inhibitor is a molecule, e.g., an antibody, protein, nucleic acid, or small molecule that inhibits TGFβ signaling activity. Typically, TGFβ inhibition binds to or blocks the TGF-β receptor (TGF-βR). Examples of TGFβ inhibitors include, but are not limited to, SB431542, A83-01, RepSox, SB525334, SB505124, LY364947, galunisertib, D4476, GW788388, SD208, R268712, IN1130, SM16, A77-01, and AZ12799734. TGFβ inhibitors can be effective in the methods provided herein at a range between 0.1 and 10 micromolar. For example, the TGFβ inhibitors described herein can be used at 0.1 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, 2.0 μM, 3 μM, 4, μM, 5 μM, 6 μM, 7 μM, 8 μM, 9 μM, 10 μM, and any concentrations in between.

### Thyroid Hormone Receptor Activator/Agonist

A thyroid hormone receptor activator is a molecule, e.g., an antibody, protein, nucleic acid, or small molecule that enhances thyroid hormone receptor signaling activity. Typically, thyroid hormone receptor agonist/activator binds to or activates the thyroid hormone receptor. Examples of thyroid hormone receptor agonist/activator activators include, but are not limited to, triiodothyronine (T3), T4, resmetirom, eprotirome, sobetirome, Sob-AM2, VK2809, MB07344, IS25 and TG68.

### Methods of Using Gata6-Positive Brown Adipose Precursors

The Gata6-positive brown adipose precursors (BAs, or BAT) produced using methods described herein can be used in a number of different ways. For example, the cells can be used in a transplantation protocol in which BAT cells are transplanted into a subject to treat metabolic disorders such as diabetes, insulin-resistance, obesity, hypertension, an insulin resistance disorder, or hepatic steatosis. The cells can optionally be derived from induced pluripotent stem (iPS) cells or embryonic stem (ES) cells, cells obtained from the subject themselves (e.g., autologous), or can be derived from allogeneic cells. The Gata6-positive brown adipose precursor cells can be transplanted into areas where BAT is already present, e.g., a supraclavicular region, the nape of the neck, over the scapula, alongside the spinal cord, near proximal branches of the sympathetic nervous system that terminate in BAT depots, around at least one of the kidneys, the renal capsule, the liver, the skin, or elsewhere. The Gata6-positive brown adipose precursors cells can be suspended in a suitable transplant media, such as phosphate buffered saline or other salines. The cell transplant mixture can be injected via a syringe with a needle ranging from 30 to 18 gauge, with the gauge of the needle being dependent upon such factors as the overall viscosity of the adipocyte suspension, into a target location. Preferably, needles ranging from 22 to 18 gauge and 30 to 27 gauge can be used. See, e.g., US20170000827; Liu et al., Cell Res 23, 851-854 (2013); and US20170014455, which are incorporated by reference in their entireties. In some embodiments, the cells are present in a biocompatible semisolid or gel matrix, e.g., a hydrogel matrix, suitable for transplantation; for example, a hyaluronic acid-based hydrogel (see Tharp et al., Diabetes. 2015 November; 64(11):3713-24); Tharp and Stahl, Front Endocrinol (Lausanne). 2015; 6: 164; Vaicik et al., J. Mater. Chem. B, 2015, 3, 7903-7911); collagen/alginate microspheres (Yao et al., Biofabrication, 4(4): 045003 (2012)); or adipose tissue derived soluble extracellular matrix (sECM) and methylcellulose (MC) (see Kim et al., PLoS ONE 11(10): e0165265) can be used. See also Cho et al., Cell Transplat 16(4):421-434 (2007). Compositions comprising these hydrogels and the Gata6-positive brown adipose precursors cells described herein are also within the scope of the present disclosure.

In some embodiments, the methods described herein include treating a subject with the Gata6-positive brown adipose precursor cells or cells derived from Gata6-positive brown adipose precursor cells for metabolic-related indications, e.g., diabetes, insulin-resistance, obesity, hypertension, an insulin resistance disorder, or hepatic steatosis. The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent maybe administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. In some embodiments, the cells would be administered by injection, graft, infusion, implant, or any combination thereof

### Methods of Screening (Test Compounds)

In addition to their use in transplantation, the Gata6-positive brown adipose precursor cells derived using the present methods can also be used, e.g., for in vitro screening of drugs to determine their effect on BAT, including thermogenic assays. Thus, included herein are methods for screening test compounds, e.g., polypeptides, polynucleotides, inorganic or organic large or small molecule test compounds, to identify agents useful in the treatment of metabolic disorders such as diabetes, insulin-resistance, obesity, hypertension, an insulin resistance disorder, or hepatic steatosis. The methods can be used to identify whether a test compound has an effect on BAT, e.g., to increase BAT numbers or activity, or to decrease BAT numbers or activity. In some embodiments, the methods used to identify whether a test compound has an effect on BAT include testing for an increase of UCP1 expression. In some embodiments, the methods used to identify whether a test compounds has an effect on BAT include testing for glycerol release. In some embodiments, the methods used to identify whether a test compounds has an effect on BAT include testing for an increase in oxygen consumption. In some embodiments, the methods used to identify whether a test compounds has an effect on BAT include testing for an increase in temperature. In some embodiments, the methods used to identify whether a test compounds has an effect on BAT include measuring metabolism and thermogenic capacity of cells including measurement of glycolysis, mitochondrial respiration, lipid synthesis, beta-oxidation and mitochondrial uncoupling. In some embodiments, the methods used to identify whether a test compounds has an effect on BAT include measuring response on RNA and protein expression of BAT specific genes.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample comprising a Gata6-positive brown adipose precursor cell or tissue a brown adipose cell obtained using a method described herein, and one or more effects of the test compound is evaluated. For example, the methods can evaluate the ability of the test compound to alter expression of one or more of UCP-1, Dio2, Cidea2, FABP4, C/ebp, Cox7a1, Cox7a2, Cox8a, Zic1, Ebf2, Cd34, and/or Prdm16, and/or proliferation of BAT progenitors, and/or fat storage evaluated for example by Oil Red O staining and lipid dye, and/or BAT thermogenic activity, and/or BAT metabolic activity, as evaluated by measure of glycolysis, mitochondrial respiration, lipid synthesis, beta-oxidation and mitochondrial uncoupling.

Methods for evaluating each of these effects are known in the art. For example, ability to modulate expression of a protein can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect an effect on the BAT. Ability to modulate signaling via the VEGFR2, atrial natriuretic peptide receptor (NPR), beta3 adrenergic receptor (03-AR), OxR1, BMPRII, Alk7 (Acvr1c), FGFR, and/or Irisin receptor signaling pathways can be evaluated, e.g., using genetically encoded reporter assays and/or using biochemical assays for known intracellular transduction pathways transduction including, p38, PKC, Pi3K, and phosphoSMADs (see, e.g., Tchivileva et al., Mol Immunol. 2009 July; 46(11-12): 2256-2266; Kumar et al., 1356(2):221-228, 1997; Moshinsky et al., J Biomol Screen. 2003 August; 8(4):447-52; Zilberberg et al., BMC Cell Biol. 2007; 8: 41; Logeart-Avramoglou et al., Anal Biochem. 2006 Feb. 1; 349(1):78-86]. In some embodiments, differentiation of iPSCs into brown adipocytes can be determined by using cells that express a reporter. For example, a UCP1-mCherry construct can be introduced immediately 5′ to the start codon of UCP1, separated by a P2A, to create a fluorescent human iPSC reporter line using CRISPR-Cas9.

Thermogenic assays can include exposing the cells to thermogenesis-inducing factors, including but not limited to a GPR120 activator (e.g., GW9508 (see Quesada-Lopez et al., Nat Commun. 2016 Nov. 17; 7:13479) or Berberine (see Zhang et al., Nat Commun. 2014 Nov. 25; 5:5493) or exposure to cold.

A test compound that has been screened by a method described herein and determined to increase expression of UCP-1, Dio2, Cidea2, FABP4, C/ebp, Ebf2, and/or Prdm16, and/or increase proliferation of BAT progenitors, and/or increase fat storage and lipolysis evaluated for example by Oil Red O staining, and/or increase BAT mitochondrial content, and/or increase BAT thermogenic activity, and/or increase BAT metabolic activity, can be considered a candidate compound for treating a metabolic disorder.

Alternatively, the methods can be used to identify compounds that decrease BAT activity, e.g., to encourage weight gain or to treat conditions associated with increased BAT activity, hypermetabolism, underweight or weight loss associated with chronic disease. A test compound that has been screened by a method described herein and determined to decrease expression of UCP-1, Dio2, Cidea2, FABP4, C/ebp, Ebf2, and/or Prdm16, and/or decrease proliferation of BAT progenitors, and/or decrease fat storage and lipolysis evaluated for example by Oil Red O staining, and/or decrease BAT mitochondrial content, and/or decrease BAT thermogenic activity, and/or decrease BAT metabolic activity, can be considered a candidate compound for treating a condition associated with increased BAT activity, hypermetabolism, underweight, or weight loss associated with chronic disease.

A candidate compound that has been screened, e.g., in an in vivo model of a disorder, e.g., a metabolic disorder such as diabetes, insulin-resistance, obesity, hypertension, an insulin resistance disorder, or hepatic steatosis, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Test compounds identified as “hits” (e.g., test compounds that increase expression of UCP-1, increase proliferation of BAT, increase Oil Red O staining, or to increase expression of UCP-1, Dio2, Cidea2, FABP4, C/ebp, Ebf2, and/or Prdm16, and/or increase proliferation of BAT progenitors, and/or increase fat storage evaluated for example by Oil Red O staining, and/or increase BAT mitochondrial content, and/or increase BAT thermogenic activity, and/or increase BAT metabolic activity) in an Gata6-positive brown adipose precursor screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating metabolic disorder such as diabetes, insulin-resistance, obesity, hypertension, an insulin resistance disorder, or hepatic steatosis. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of metabolic disorder such as diabetes, insulin-resistance, obesity, hypertension, an insulin resistance disorder, or hepatic steatosis, as described herein. The animal can be monitored for a change in the disorder, e.g., for an improvement in a parameter of the disorder, e.g., a parameter related to clinical outcome. In some embodiments, the parameter is weight, and an improvement would be weight loss. In some embodiments, the subject is a human, e.g., a human with obesity, and the parameter is weight loss. In some embodiments, the subject is a human with, or an animal model of, a metabolic disorder such as diabetes, insulin-resistance, hypertension, an insulin resistance disorder, or hepatic steatosis, and the parameter is improved blood glucose control, improved insulin sensitivity, improved blood pressure control or improved liver enzyme levels. In this context, “improved” means returned to or near normal levels.

### EXAMPLES

The invention is further described in the following examples, which do not limit the scope of the invention described in the claims.

### Experimental Procedure

### Human Induced Pluripotent Stem Cell Maintenance

NCRM1 (NIH CRM control iPSC line (male)) human iPSC and other cell lines were routinely cultured on Matrigel (Corning, 354263) coated culture plates (Corning, 353046) in mTeSR (Stemcell Technologies, 85850). Upon confluency, cultures were dissociated into single cells using Accutase (Corning, 25-058-C). Cells were seeded on Matrigel coated culture plates in mTeSR supplemented with 10 μM Y-27632 dihydrochloride (R&D Systems, 1254/10) at a density of 56,000/cm2 for routine maintenance of iPSC lines. On the following day, medium was replaced with mTeSR only and medium was changed every day. Cultures became confluent every fourth day and were passaged as described above. For freezing, cultures were dissociated into single cells with Accutase and frozen in NutriFreez D10 Cryopreservation Medium (Biological Industries, 01-0020-50).

### Differentiation

iPSCs were differentiated into presomitic mesoderm as described previously (Chal et al., 2016). Briefly, confluent maintenance cultures were dissociated into single cells using Accutase (Corning, 25-058-C) and cells were seeded at a density of 30,000-33,000/cm2 on Matrigel (Corning, 354263) coated culture plates (Corning, 353046) in mTeSR (Stemcell Technologies, 85850) supplemented with 10 μM Y-27632 dihydrochloride (R&D Systems, 1254/10). Next day, the cells formed small compact colonies. On day 0 of differentiation, to induce presomitic mesoderm, cells were treated with CL medium [DMEM/F12 GlutaMAX (Thermo Fisher Scientific, 10565042)+1% Insulin-Transferrin-Selenium (Gibco, 41400045)+3 μM CHIR 99021 (R&D Systems, 4423)+0.5 μM LDN-193189 (Stemgent, 04-0074)]. Cells were treated with CL medium for 3 days and medium was refreshed every day. On day 3 of differentiation, cells were changed to CLF medium [CL medium+20 ng/ml FGF-2 (PeproTech, 450-33)]. Cells were treated with CLF medium for 3 days and medium was changed every day.

To further differentiate presomitic mesoderm cells to dermomyotomal fate, cells were changed to HIFL medium [DMEM high glucose (Thermo Fisher Scientific, 11965-118)+Penicillin/Streptomycin (Life Technologies, 15140122)+15% KnockOut™ Serum Replacement (Life Technologies, 10828-028)+NEAA (Thermo Fisher Scientific, 11140-050)+0.01 mM 2-Mercaptoethanol (Life Technologies, 21985-023)+10 ng/ml HGF (PeproTech, 315-23)+2 ng/ml IGF-1 (PeproTech, 250-19)+20 ng/ml FGF-2 (PeproTech, 450-33)+0.5 μM LDN-193189 (Stemgent, 04-0074)]. Cells were treated with HIFL for two days and medium was changed every day. To let the PAX3 expressing dermomyotomal cells into myogenic and non-myogenic lineages, cells were changed to HI medium (HIFL medium without FGF-2 and LDN-193189) and medium was changed every day.

On day 16-30 of differentiation, cells were replated to differentiate cells into brown adipocyte lineage. Cultures were dissociated into single cells with 2.5 mg/ml Collagenase, (Type IV, Thermo Fisher Scientific, 17104019) and 0.05% Trypsine EDTA (Thermo Fisher Scientific, 25200-056) in PBS (Gibco, 14190) and filtered through a 30 μm (CellTrics, 04-0042-2316) filter and seeded on Matrigel coated plates at a density of 60,000-100,000/cm2 in BCTFT medium [DMEM high glucose (Thermo Fisher Scientific, 11965-118)+Penicillin/Streptomycin (Life Technologies, 15140122)+5% KnockOut™ Serum Replacement (Life Technologies, 10828-028)+1% Insulin-Transferrin-Selenium (Gibco, 41400045)+10 ng/ml FGF-2 (PeproTech, 450-33)+10 ng/ml BMP7 (Thermo Fisher Scientific, PHC9544)+20 nM Porcn Inhibitor II, C59 (Millipore Sigma, 500496)+10 ng/ml TGFb1 (PeproTech, 100-21-10)+2 nM 3,3′,5-Triiodo-L-thyronine sodium salt (Sigma-Aldrich, T6397)]. Cells were treated with BCTFT for 4-6 days and medium was refreshed every day. Adipocyte precursors were either frozen to be used later or differentiated into adipocytes. For freezing, cultures were dissociated into single cells using 0.05% Trypsine EDTA (Thermo Fisher Scientific, 25200-056) in PBS (Gibco, 14190) and cells were frozen in NutriFreez D10 Cryopreservation Medium (Biological Industries, 01-0020-50).

To differentiate adipocyte precursors into adipocytes, cells were cultured in adipogenic medium [DMEM high glucose (Thermo Fisher Scientific, 11965-118)+Penicillin/Streptomycin (Life Technologies, 15140122)+5% KnockOut™ Serum Replacement (Life Technologies, 10828-028)+1% Insulin-Transferrin-Selenium (Gibco, 41400045)+500 μM IBMX (3-Isobutyl-1-methylxanthine, Sigma-Aldrich, 17018)+25.5 g/ml L-Ascorbic acid (Sigma-Aldrich, A4544)+2 nM T3 (3,3′,5-Triiodo-L-thyronine sodium salt, Sigma-Aldrich, T6397)+5 μM TGFb inhibitor SB431542 (Selleck Chemicals, S1067)+1 μM Dexamethasone (Sigma-Aldrich, D4902)+10 ng/ml EGF (Peprotech, AF-100-15)+4 g/ml Hydrocortisone (Sigma-Aldrich, H0888)+1[M Rosiglitazone (Sigma-Aldrich, R2408)] for 30-40 days. Medium was changed every third day.

To perform functional assays and immunofluorescence analysis, 30-40 day old replated cultures were dissociated with 2.5 mg/ml Collagenase, (Type IV, Thermo Fisher Scientific, 17104019) and 0.05% Trypsine EDTA (Thermo Fisher Scientific, 25200-056) in 1× Phosphate Buffered Saline (Gibco, 14190) and cells were seeded onto suitable plate format required for the assay.

### Human Fetal Tissue

Human fetal tissues were obtained by the University of Washington Birth Defects Research Laboratory (BDRL) under a protocol approved by the University of Washington Institutional Review Board. BAT tissues were isolated at 98 days (H28540), 115 days (H28572), 122 days (H28560), 125 days (H28626) and 135 days estimated post-conceptual age from the interscapular and scapular region. Tissues were rinsed in 1× Hanks' Balanced Salt Solution (HBSS, Thermo Fisher Scientific, 14185052) before processing. For immunofluorescence analysis, tissues were fixed with 4% Paraformaldehyde (Electron Microscopy Sciences, 15710) overnight at 4° C., washed 3 times in 1× Phosphate Buffered Saline (PBS, Sigma-Aldrich, P5493) and stored at 4° C. in PBS supplemented with 0.1% Sodium Azide (Sigma-Aldrich, 71290) until further processing. For bulk mRNA sequencing, tissues were snap frozen in liquid nitrogen and stored at −80° C. until further processing.

### Mouse Lines

Pax7-iCre/+; Rosa26-mTmG/+ embryos were generated by crossing Rosa26-mTmG/mTmG (Muzumdar et al., 2007) males with Pax7-iCre/+ females in timed matings and embryos were collected at E14.5. Gata6-EGFPCreERT2 mice were generated by knocking an EGFPCreERT2 cassette into the endogenous Gata6 locus as reported previously (Donati et al., 2017). They were crossed with Rosa26-fl/STOP/fl-tdTomato mice (Madisen et al., 2010). For lineage tracing experiments, pregnant females were injected intraperitoneally with 50 lg/g of tamoxifen (Sigma) at E12.5 and embryos were collected at E15.5. For experiments other than lineage tracing, wildtype CD1 IGS mice (Charles River) were used.

### Generation of UCP-mCherry and PAX3-Venus Reporter Line

UCP1 gene was targeted using the CRISPR-Cas9 system-based genome editing to generate a reporter NCRM1 iPSC line which already had been modified at PAX7 locus to introduce Venus. To target the UCP1 locus, a single guide RNA (sense strand encoding gRNA=CACCGGGTTTGCTGCCCGGCGGAC (SEQ ID NO: 1), antisense strand encoding gRNA=AAACGTCCGCCGGGCAGCAAACCC) (SEQ ID NO: 2) targeting the 5′ region of the gene was designed using the MIT Crispr Design Tool (crispr.mit.edu). The guide RNA was cloned into PSpCas9 (BB)-2A-GFP(PX458) (Addgene, 48138) following protocol from Ran et al., 2013. The final vector was sequence to ensure no mutations were generated during cloning. To generate a targeting vector for homology dependent repair, we cloned 5′ and 3′ 1 kb long homology sequence (HA) flanking a nuclear localization sequence (NLS) region from H2B gene sequence, fluorescent protein mCherry sequence and self-cleaving P2A peptide sequence (5′HA-H2B-mCherry-P2A-3′HA) in a pUC19 vector backbone using Gibson Assembly (New England Biolabs (NEB), E5510S). To mutate the PAM site for the guide RNA, the assembled targeting vector was mutated using site-directed mutagenesis using In-Fusion HD Cloning Plus (Takara, 638909). NCRM1 iPSCs were transfected with both Guide RNA vector (PSpCas9 (BB)-2A-GFP) and targeting vector (pUC19-5′HA-H2B-mCherry-P2A-3′HA) using Lipofectamine™ Stem Transfection Reagent (Thermo Fisher Scientific, STEM00001). 24-hours after transfection, cells were sorted using flow cytometry (S3 cell sorter, Biorad) for GFP positive cells and plated at low density for clonal expansion in Matrigel (Corning, #354263) coated culture plates (Corning, 353046) in mTeSR (Stemcell Technologies, 85850) supplemented with 10 μM Y-27632 dihydrochloride (R&D Systems, 1254/10) and Penicillin/Streptomycin (Life Technologies, 15140122). After appearance of small colonies, the colonies were sub-cultured and genotyped using PCR for targeted homozygous insertion of the H2B-mCherry-P2A in the UCP1 locus before the transcription start site. Positive clones were sequenced and clones with no undesired mutation were further validated using immunofluorescence and RT-qPCR. Two positive clones were differentiated into adipocytes and expression of UCP1 in mitochondria and mCherry in the nucleus was confirmed. To generate the human PAX3-Venus iPSC line using the CRISPR/Cas9 technology we followed a similar strategy. We generated a Venus knock-in allele by inserting the Venus sequence in front of the coding sequence of exon 1. Guide RNA (sense strand encoding gRNA=5′-CCGGCCAGCGTGGTCATCCTGGG-3′ (SEQ ID NO: 3), antisense strand encoding gRNA=5′-TGCCCCCAGGATGACCACGCTGG-3′ (SEQ ID NO: 4)) targeting the 5′ region of the gene was designed using the MIT Crispr Design Tool (crispr.mit.edu). The targeting plasmid (pBSKS-2A-3xNLS) was designed to contain NLS sequence, 1.5 kb of the 5′ genomic region of the PAX3 gene, 1 kb of the 3′ sequence and 2A sequence. Targeting vector, together with the Cas9 plasmid was electroporated into cells by nucleofection and clones were sub-cultured and genotyped using PCR for targeted homozygous insertion of the NLS-Venus-2A in the PAX3 locus.

### RNA Extraction, Reverse Transcription and Real Time Quantitative PCR

Samples were harvested and RNA was extracted using NucleoSpin® RNA kit (Macherey and Nagel, 740955) following manufacturer's instructions. DNA digestion was performed on column and RNA quality and concentration was measured using Nanodrop. For reverse transcription, lug RNA was in a 20 ul reaction volume to generate cDNA using iScript™ cDNA Synthesis Kit (Bio-Rad, 1708891). Typically, cDNA was diluted 1:10 with nuclease free water. For real time quantitative PCR, 3.5 ul of cDNA, 1.5 ul of 10 μM forward and reverse primer mix (300 μM of forward and reverse primers) and 5 ul of iTaq™ Universal SYBR® Green Supermix (Bio-Rad, 172-5124) was used for 10 ul reaction volume. For each sample and gene primer set, 3 technical replicates were performed. PCR primers were designed using primer 3, typically spanning splice junctions wherever possible. Primers were validated for amplification efficiency and specificity using a standard curve and melting curves, respectively. PCRs were run on a Bio-Rad CFX384 thermocycler with the following cycling program: initial denaturation step (95° C. for 1 minute), 40 cycles of amplification and SYBR green signal detection (denaturation at 95° C. for 5 seconds, annealing/extension and plate read at 60° C. for 40 seconds), followed by final rounds of gradient annealing from 65° C. to 95° C. to generate dissociation curves. Relative gene expression was calculated using ΔΔCt method and RPL37A (ribosomal protein L37a) was used as a housekeeping gene. A list of qPCR primers is provided.

### RT-qPCR Primers:

| Gene | forward primer | reverse primer |
|---|---|---|
| ADIPOQ | CTCGAACTCCTGACCTTGT | CACAAGGCTTACTCTCACCTC |
| | GATCT (SEQ ID NO: 5) | TTCA (SEQ ID NO: 6) |
| CIDEA | GACGGGACAGTTTCTCATC | ATCCCTATCCACACGTGAACC |
| | TATCT (SEQ ID NO: 7) | T (SEQ ID NO: 8) |
| EBF2 | TAACGAGGACAGGGTAGC | CATCTTGACCATTCAGCTAGA |
| | TTTGT (SEQ ID NO: 9) | ACC (SEQ ID NO: 10) |
| FABP4 | CCACAGAATGTTGTAGAGT | TTCCACGAGAGTTTATGAGAG |
| | TCAATGC (SEQ ID NO: 11) | AGCA (SEQ ID NO: 12) |
| GATA6 | GGATGTCCTATGGAAACCT | GAAGGAACTCACTGTACAGA |
| | ATTTCA (SEQ ID NO: 13) | AGACAAA (SEQ ID NO: 14) |
| MYOG | GAAGAGACCAGAACAGGA | TTTATCATAACGCGGCCTCTA |
| | GACGTG (SEQ ID NO: 15) | ATTC (SEQ ID NO: 16) |
| PGC1A | ACATACATGCACACACGCA | GCTGAAGAGGCAAGAGACAG |
| | CACT (SEQ ID NO: 17) | AATG (SEQ ID NO: 18) |
| PLIN1 | AGGAGTTCAGTGCTAAGA | TGAGTAACTCCTGTCACCACT |
| | ATGTGTCAA (SEQ ID NO: | CTGAA (SEQ ID NO: 20) |
| | 19) | |
| PPARG | GATATCGACCAGCTGAATC | TCAGCGGGAAGGACTTTATGT |
| | CAGAG (SEQ ID NO: 21) | ATG (SEQ ID NO: 22) |
| PRDM16 | AGGGAATGGATAGACTGG | ACTCTGCAGTCAATACGGTTA |
| | TGTGCT (SEQ ID NO: 23) | CCAAA (SEQ ID NO: 24) |
| RPL37A | GTGGTTCCTGCATGAAGAC | TTCTGATGGCGGACTTTACCG |
| | AGTG (SEQ ID NO: 25) | (SEQ ID NO: 26) |
| UCP1 | TGAAGCTGATTATGTGGCA | GACTTGGATCCTGGAACGTCA |
| | CAGTC (SEQ ID NO: 27) | TTA (SEQ ID NO: 28) |

### Cultured Cells:

Cells were cultured on Matrigel coated plastic culture plates (Corning, #353046), glass bottom 24-well plates (Cellvis, P24-1.5H-N), μ-Dish 35 mm (Ibidi, 81156) or μ-Plate 24 Well Black (Ibidi, 82406). Cultures were washed with 1× Phosphate Buffered Saline (PBS, Sigma-Aldrich, P5493) and fixed with 400 Paraformaldehyde (Electron Microscopy Sciences, 15710) for 20 minutes at room temperature. Cultures were washed with PBS and stored in PBS supplemented with 0.2% Sodium Azide (Sigma-Aldrich, 71290) until used for immunostaining.

### Mouse Tissues:

Mouse embryos were dissected out at different stages in Hanks' Balanced Salt Solution (HBSS) (Gibco, 14185-052). After several washes in HBBS, embryos were fixed in 4% Paraformaldehyde (Electron Microscopy Sciences, 15710) overnight at 4° C. Embryos were then washed in PBS and transferred to 15% sucrose solution (Sigma-Aldrich, 84097) in PBS overnight at 4° C. followed by incubation in 30% sucrose solution for overnight at 4° C. Embryos were then embedded in Tissue-Tek O.C.T. Compound (VWR, 25608-930) and stored at −80° C. until sectioned. 10-20 m thick sections were cut and stored at −20° C. until used for immunostaining.

Fixed cells or sections were permeabilized using 1% Triton-X (Millipore Sigma, T8787) for 10 minutes at room temperature. Samples were washed in PBS and were incubated in blocking solution [PBS supplemented with 3% donkey serum (Jackson ImmunoResearch, 017-000-121) and 0.1% Triton-X]for 1 hour at room temperature. Samples were then incubated with primary antibody diluted in blocking solution at 4° C. overnight. Next day, samples were washed 3 times with PBS for 5 minutes. Samples were then incubated with secondary antibody and Hoechst 33342 (ThermoFisher Scientific, H3570) diluted in blocking solution for 1 hour at room temperature followed by 3 washes with PBS for 5 minutes each. Cultured cell samples were stored in PBS until imaged. Mouse tissue slides were mounted using Fluoromount aqueous mounting medium (Sigma-Aldrich, F4680) and were stored at 4° C. until analyzed. Samples were imaged using EVOS FL imaging system (Thermo Fisher Scientific) or LSM780 confocal microscope (Zeiss). A list of primary and secondary antibodies is provided.

### Primary Antibodies:

| Antigen | Antibody company/catalog number | Host | dilution |
|---|---|---|---|
| Cd34 | ThermoFisher/14-0341-82 | Rat | 1/100 |
| Dpp4 | R&D/AF954-SP | Goat | 1/100 |
| EBF-2 | R&D/AF7006 | Sheep | 1/500 |
| Gata6 | RnD/AF1700-SP | Goat | 1/100 |
| Gata6 | Cell Signaling Technology/5851T | Rabbit | 1/100 |
| GFP | SantaCruz/SC-101536 | Rat | 1/1000 |
| GFP | Abcam/AB13970 | Chicken | 1/1000 |
| Keratin 14 | Biolegend/906001 | Chicken | 1/100 |
| mCherry | OriGene/AB0040-200 | Goat | 1/100 |
| mCherry | Neuromics/CH22115 | Chicken | 1/500 |
| MTC02 | Abcam/AB3298 | Mouse | 1/250 |
| Myh11 | Alfa Aesar/J64817 | Rabbit | 1/100 |
| MyHCfast | Sigma-Aldrich/M4276 | Mouse | 1/100 |
| Perilipin 1 | Progen/GP29 | Guinea pig | 1/200 |
| Perilipin-1 | Abcam/AB61682 | Goat | 1/200 |
| PPARG | ThermoFisher/MA5-14889 | Rabbit | 1/100 |
| RFP | Rockland/600-901-379 | Chicken | 1/1000 |
| RFP | Rockland/600-401-379 | Rabbit | 1/1000 |
| Sca-1/Ly6 | R&D/AF1226-SP | Goat | 1/100 |
| TBX6 | Abcam/AB38883 | Rabbit | 1/100 |
| UCP1 | R&D/MAB6158 | Mouse | 1/100 |

### Secondary Antibodies:

| | Company/Catalog | |
|---|---|---|
| Antibody | number | dilution |
| Donkey Anti-Rat IgG H&L (Alexa Fluor ® | Abcam/ab150155 | 1/500 |
| 647) preadsorbed | | |
| Donkey anti-Goat IgG (H + L) Secondary | Thermo Fisher | 1/500 |
| Antibody, Alexa Fluor ® 488 conjugate | Scientific/A-11055 | |
| Donkey anti-Goat IgG (H + L) Cross-Adsorbed | Thermo Fisher | 1/500 |
| Secondary Antibody, Alexa Fluor 647 | Scientific/A-21447 | |
| Donkey anti-Sheep IgG (H + L) Cross- | Thermo Fisher | 1/500 |
| Adsorbed Secondary Antibody, Alexa Fluor | Scientific/A-11016 | |
| 594 | | |
| Donkey anti-Sheep IgG (H + L) Secondary | Life Technologies/A- | 1/500 |
| Antibody, Alexa Fluor ® 488 conjugate | 11015 | |
| Donkey anti-Rabbit IgG (H + L) Highly Cross- | Thermo Fisher | 1/500 |
| Adsorbed Secondary Antibody, Alexa Fluor | Scientific/A21206 | |
| 488 | | |
| Donkey anti-Rabbit IgG (H + L) Secondary | Thermo Fisher | 1/500 |
| Antibody, Alexa Fluor ® 594 | Scientific/A-21207 | |
| Donkey anti-Rabbit IgG (H + L) Secondary | Thermo Fisher | 1/500 |
| Antibody, Alexa Fluor ® 647 | Scientific/A-31573 | |
| Donkey anti-Rabbit IgG (H + L) Highly Cross- | Thermo Fisher | 1/500 |
| Adsorbed Secondary Antibody, Alexa Fluor | Scientific/A-31572 | |
| 555 | | |
| Alexa Fluor ® 488 AffiniPure Donkey Anti- | Jackson ImmunoResearch | 1/500 |
| Chicken IgY (IgG) (H + L) | Laboratories/703-545-155 | |
| Cy ™3 AffiniPure Donkey Anti-Chicken IgY | Jackson ImmunoResearch | 1/500 |
| (IgG) (H + L) | Laboratories/703-165-155 | |
| Donkey anti-Mouse IgG (H + L) Highly Cross- | Thermo Fisher | 1/500 |
| Adsorbed Secondary Antibody, Alexa Fluor | Scientific/A-31571 | |
| 647 | | |
| Alexa Fluor ® 488 AffiniPure Donkey Anti- | Jackson ImmunoResearch | 1/500 |
| Guinea Pig IgG (H + L) | Laboratories/706-545-148 | |

### Lipid Staining

iPSC-BA cultures were washed with 1× Phosphate Buffered Saline (PBS, Sigma-Aldrich, P5493) and fixed with 4% Paraformaldehyde (Electron Microscopy Sciences, 15710) for 20 minutes at room temperature. Cell were then incubated with 0.5 mM BODIPY™ (Invitrogen™, D3922) in PBS for 20 minutes at room temperature. Cells were washed 3 times with PBS and visualized with EVOS FL imaging system (Thermo Fisher Scientific).

### Seahorse Assay

Agilent Seahorse XFe96 Analyzer was used to measure oxygen consumption in live iPSC-BAs and precursor cells following manufacturer's instructions. 30,000 iPSC-BAs and precursor cells were cultured on Matrigel (Corning, 354263) coated Seahorse XF96 cell culture microplates (Agilent Technologies, 101085-004). Assay medium was prepared using Seahorse XF DMEM (Agilent Technologies, 103575-100) supplemented with 1 mM pyruvate (Agilent Technologies, 103578-100), 2 mM glutamine (Agilent Technologies, 103579-100), and 10 mM glucose (Agilent Technologies, 103577-100). Basal respiration was measured 3 times in the assay medium. Cells were then treated with 1.5 μM Oligomycin (Tocris, 4110) and 3 measurement were taken. Cells were then treated with 10 μM Forskolin (Sigma-Aldrich, F6886) or DMSO (Sigma-Aldrich, D2650) and oxygen consumption rate (OCR) was measured for 70 minutes (12 measurements). To block the electron transport chain, cells were then treated with 1p M Rotenone (Sigma-Aldrich, R8875) and Antimycin A (Sigma-Aldrich, A8674) for 3 measurements. Cells were then washed with 1× Phosphate Buffered Saline (PBS, Sigma-Aldrich, P5493) and fixed with 4% Paraformaldehyde (Electron Microscopy Sciences, 15710) for 10 minutes at room temperature. Seahorse XF96 cell culture microplates were imaged for DAPI and mCherry using GE INCELL Analyzer 2200 Widefield High-Content Imager. Cells were automatically segmented using custom code in Fiji and number of DAPI and mCherry positive cells were quantified. OCR data from each well was normalized with DAPI positive cell number in each well. Four replicates were performed. Each experiment consisted of 40 technical replicates for each cell type.

### Thermogenesis Assay

iPSC-BAs and iPSC precursors were cultured on Matrigel (Corning, 354263) coated 96-well Black Clear-Bottom Plates (Corning, 3603) and were incubated in DMEM/H with 250 nM ERthermAC (Sigma, SCT057) for 30 min at 37° C. After washing with PBS, fresh 90 μl DMEM/H without phenol red was added prior to imaging. Fluorescence in stained cells were detected with a GloMax Discover Multimode Detection System (Promega) using 520 nm excitation and emission at 590 nm. Temperature inside the machine was equilibrated at 25° C. After measuring 3 points of basal fluorescence, 10 μl forskolin (final concentration 10 μM, F6886, Sigma) was added to initiate thermogenesis. 10 μl PBS was added for vehicle control group. Fluorescence was recorded every 5 min over 120 min. Results are interpreted as relative intensity (intensity was normalized to basal measurements).

### Electron Microscopy

iPSC-BAs were rinsed with 0.1M Phosphate buffer pH 7.5 and fixed in 2.5% glutaraldehyde (Sigma, G7651) in 0.1M phosphate buffer at 4° C., for 2 days. Samples were washed in 0.1M phosphate buffer and post-fixed in 1% osmic acid in 0.1M phosphate buffer for 1 hour at room temperature. Dehydration was performed by serial incubation in 50%, 70%, 80%, 95%, and 100% ethanol before incubation in propylene oxide for 30 minutes. Samples were impregnated by incubation in propylene oxide/araldite (1:1 v/v) for 60-90 min, in propylene oxide/araldite (1:2 v/v) for 1 hour and in 100% araldite overnight at 4° C. The samples were finally incubated in Araldite which was allowed to polymerize for 24 hours at 56° C. Ultrathin sections (85 nm) were cut with a ultracut Leica EM UC. Sections were contrasted 8 min with 2% uranyl acetate and 8 min with lead citrate (Reynolds). All pictures were taken with the Zeiss EM 900 microscope and a GATAN camera (Orius SC 1000).

### Glycerol Release Assay

iPSC-BAs were cultured in a (Corning, 354263) coated 96-well culture plate (Genesee Scientific, 25-109). Cells were serum starved for 16 hours and cultured in serum-free DMEM/F12 GlutaMAX (Thermo Fisher Scientific, 10565042) containing 0.5% BSA. Next day, cells were washed with KRB-HEPES buffer [118.5 mM NaCl, 4.75 mM KCl, 1.92 mM CaCl₂), 1.19 mM KH2PO4, 1.19 mM MgSO4, 25 mM NaHCO₃, 6 mM glucose and 10 mM HEPES, pH 7.4] containing 4% fatty-acid-free BSA (Bioworld, 22070017-1). Cells were treated with DMSO or 10 μM Forskolin in KRB-HEPES buffer supplemented with 4% fatty-acid-free BSA for at 37° C., 5% C02 for 4 hours. Cell culture medium was collected for glycerol measurement using the free glycerol reagent (Sigma-Aldrich, F6428) following manufacturer's instructions. A standard curve was generated using Glycerol Standard Solution (Sigma-Aldrich, G7793). Test samples, standards and water control were incubated with the Free Glycerol Reagent for 5 minutes at 37° C. and absorbance was recorded at A540 using a spectrophotometer. To normalize with total protein content, Bradford assay was performed using DC Protein Assay (Biorad, 500-0116) following manufacturers protocol.

### Periodic Acid-Schiff Staining

To stain glycogen in differentiating iPSC-BAs, cells were treated with Periodic Acid-Schiff stain (Sigma, 395B). iPSC-BAs were fixed with 4% paraformaldehyde (Electron Microscopy Sciences, 15710) at room temperature for 15 minutes. Fixed cells were washed several times with distilled water and immersed in Periodic Acid Solution for 5 minutes at room temperature. Cells were then washed in distilled water and incubated with Schiff's Reagent for 15 minutes at room temperature. Cells were washed in running tap water for 5 minutes. Stained cells were visualized in brightfield using EVOS FL imaging system (Thermo Fisher Scientific).

Bulk mRNA Sequencing

UCP1-mCherry knock-in line was differentiated into brown adipocytes as described above. Cells were harvested on day +40 of differentiation and RNA was extracted using NucleoSpin® RNA kit (Macherey and Nagel, 740955) following manufacturer's instructions. DNA digestion was performed on column and RNA quality and concentration was measured using Nanodrop. Human fetal tissues were harvested as described above and RNA was extracted using NucleoSpin® RNA kit following manufacturer's instructions.

RNA library preparations and sequencing reactions were conducted at GENEWIZ, LLC. (South Plainfield, NJ, USA). RNA samples received were quantified using Qubit 2.0 Fluorometer (Life Technologies, Carlsbad, CA, USA) and RNA integrity was checked using Agilent TapeStation 4200 (Agilent Technologies, Palo Alto, CA, USA). RNA sequencing libraries were prepared using the NEBNext Ultra II RNA Library Prep Kit for Illumina using manufacturer's instructions (NEB, Ipswich, MA, USA). Briefly, mRNAs were initially enriched with Oligod(T) beads. Enriched mRNAs were fragmented for 15 minutes at 94° C. First strand and second strand cDNA were subsequently synthesized. cDNA fragments were end repaired and adenylated at 3′ends, and universal adapters were ligated to cDNA fragments, followed by index addition and library enrichment by PCR with limited cycles. The sequencing library was validated on the Agilent TapeStation (Agilent Technologies, Palo Alto, CA, USA), and quantified by using Qubit 2.0 Fluorometer (Invitrogen, Carlsbad, CA) as well as by quantitative PCR (KAPA Biosystems, Wilmington, MA, USA).

The sequencing libraries were clustered on a single lane of a flowcell. After clustering, the flowcell was loaded on the Illumina HiSeq instrument (4000 or equivalent) according to manufacturer's instructions. The samples were sequenced using a 2×150 bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification.

For bulk RNA-seq data analysis, we performed quality control on the sequence data (FastQ files) using FastQC (v0.11.9) and used STAR RNA-seq aligner (v2.7.3a) (Dobin et al., 2013) to map the sequenced reads to the human reference genome (GRCh38 release 101 from ENSEMBL). Mapped reads were quantified using featureCounts (v2.0.1) (Liao et al., 2014). Starting from the raw gene counts, normalization and differential expression analysis were done in the R environment (v3.6.0), using the DESeq2 package (v 1.22.2) (Love et al., 2014). Genes were defined as differentially expressed when the false discovery rate was lower than 0.05. Gene Ontology (GO) enrichment analysis was performed on the differentially expressed genes using EnrichR (Kuleshov et al., 2016), choosing the WikiPathway 2019 Human resource.

To quantify the thermogenic potential of each sample we relied on the ProFAT webtool (Cheng et al., 2018). Starting from the raw count matrix, barplot and heatmap, representing the adipose browning capacity of each sample, were generated as standard output of ProFAT.

To produce the heat-map for genes of interest (linked to muscle, pluripotency and brown adipose tissue) we used normalized read counts produced by DESeq2. To calculate up- or down-regulation, we computed the log difference of the average of the biological replicates against the baseline value (average over all conditions) for each gene.

### Single Cell RNA Sequencing

### Preparation of Single Cell Suspension

Single cell analysis of differentiating iPSCs and mouse embryos cells was performed using inDrops as previously reported (Klein et al.).

UCP1-mCherry iPSC line was differentiated into brown adipocytes as described above. Cultures were harvested on day 20 of the primary differentiation and day 20 and 40 after replating.

The cultures were washed with PBS (Gibco, 14190) and dissociated into single cells using 2.5 mg/ml Collagenase, (Type IV, Thermo Fisher Scientific, 17104019) and 0.05% Trypsine EDTA (Thermo Fisher Scientific, 25200-056) in PBS for 5-15 minutes at 37° C. Dissociated cells were run through 70 μm cell strainer (Celltreat, 229483) followed by 30 μm cell strainer (CellTrics, 04-0042-2316), spun at 300 g for 5 minutes and resuspended in DMEM (Thermo Fisher Scientific, 11965-118) +5% fetal bovine serum (VWR, 89510-186). Cells were again spun at 300 g for 5 minutes and resuspended in DMEM+5% fetal bovine serum, this was repeated 2-3 times to remove debris. Final resuspension was made in PBS+0.1% bovine serum albumin (Gibco, 15260-037). Cell density was adjusted to 200,000 cells/ml. For day 20, 2500 cells were collected from each sample. For samples 20 day after replating and 40 day after replating, at least 3000 cells were collected from each sample from two independent differentiations.

For analyzing mouse embryonic tissues, embryos were collected on embryonic day (E) 11.5, 12.5, 13.5, 14.5 and 15.5 from wildtype CD1 IGS mice (Charles River). For each stage, back tissue dorsal to rib cage at the level of forelimb was dissected out. Neural tube/spinal cord and dorsal root ganglion were removed wherever possible. For E15.5, epidermis and underlying dermis was removed before dissociating the tissue. Dissected tissues were washed several times in Hanks' Balanced Salt Solution (HBSS, Thermo Fisher Scientific, 14170112). Tissues were transferred to microfuge tube with 200 μl of 2.5 mg/ml Collagenase, (Type IV, Thermo Fisher Scientific, 17104019) and 0.05% Trypsine EDTA (Thermo Fisher Scientific, 25200-056) in PBS (Gibco, 14190), chopped into small pieces using scissors and incubated at 37° C. for 15 minutes with intermittent shaking. Tissues were mechanically dissociated by triturating several times using wide-bore 1 ml pipette. The resulting cell suspension was mixed with HBBS+10% FBS, filtered through a 30 μm cell strainer and spun at 300 g for 5 minutes. To remove red blood cells, cell pellet was resuspended and incubated with RBC Lysis Solution (Qiagen, 158902) for 5 minutes at room temperature. Cells were washed with HBSS+10% FBS for 2-3 times to remove debris. Final suspension was made in PBS+0.1% BSA. Cell density was adjusted to 200,000/ml. For each stage, cells were collected from two littermate embryos. For E11.5-14.5 embryo, 3000 cells were encapsulated and sequenced from each embryo. For E15.5, 5000 cells were collected and sequenced from each embryo.

### Encapsulation, Sequencing, and Analysis

Single cells were encapsulated using inDrops technique as reported previously (Klein et al.). Cells were barcoded using v3 sequencing adapters and were sequenced on an Illumina NextSeq 500 using the NextSeq 75 High Output Kits using standard Illumina sequencing primers and 61 cycles for Read1, 14 cycles for Read2, 8 cycles each for IndexRead1 and IndexRead2. Sequence FASTQ files were processed according to indrops.py pipeline (available at github.com/indrops/indrops). Single cell transcriptomes were mapped to mouse (GRCm38/mm10) and human (GRCh38/hg19) reference transcriptomes. Samtools version 1.3.1, rsem version 1.3.0 and Bowtie version 1.2.2 was used with parameter -e 100.

A weighted histogram of transcript counts per cell barcode vs cell barcode abundance was used to identify transcripts originating from abundant cell barcodes. Only transcript counts originating from abundant cell barcodes were included in downstream analysis. Basic filtering parameters were used to exclude cells expressing <500 genes and genes expressed in less than 3 cells. The filtered counts were normalized by total number of counts for each biological sample. Top 1000 variable genes were identified according to Satija 2015. Cell doublets were identified using Scrublet and filtered out (PMID: 30954476). For each cell, fraction of counts due to mitochondrial genes was determined and cells with high fraction were filtered out. The cell cycle was scored as in Tirosh et al. 2016 (PMID: 27806376). Each cell was given a cell cycle score based on the expression of G2/M and S phase markers. The cells not expressing the markers from G2/M and S phase were identified to be in G0/G1 stage. Source of variation between the libraries were regressed out using bkknn batch correction function (PMID: 31400197). Single cell data were projected into a low dimensional space by principal component analysis (PCA). UMAP (McInnes et al., 2018) was used to embed the neighborhood graph. Cell clusters were identified using Leiden graph-clustering method [PMID: 30914743, Blondel et al, 2008]. Differentially expressed genes were identified by a Wilcoxon rank-sum test by comparing cells of each cluster with cells of all the other clusters.

Tracksplot were made using each genes are plotted as a filled line plot where the y are the genes processed expression values and x is each of the cells (Wolf et al., 2018). Stream: Stream pipeline was used to layout the trajectories inside each datasets. Original code and documentation: (Chen et al., 2019) Neural cells were removed from the dataset analyzed with stream as there developmental origin differ from the rest of the clusters.

Trajectory inference analysis was done using Cellrank (Lange et al., 2022). Waddington-OT kernel (Schiebinger et al., 2019) was used to infer the temporal couplings of cells from the different samples collected independently at various timepoints. Transition matrices were calculated to show the amount of mass transported from a cell type to another from a start and an end point.

Single cell RNA sequencing data from mouse E18 periaortic (pVAT) brown adipose tissue described in Angueira et al. 2021 was processed through the same processing pipeline as described above. Cell state predictions on single cell clusters from pVAT described in Angueira et al. 2021 were made using a kNN-classifier trained on our E13.5-E15.5 mouse single cell clusters (Diaz-Cuadros et al., 2020).

### Example 1: Single-Cell Transcriptomic Analysis of the Dorsal Trunk of Mouse Embryos Captures the Development of Somitic Lineages

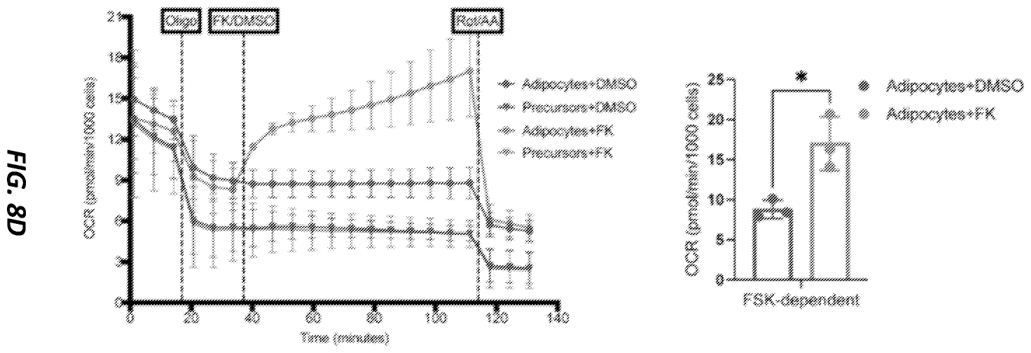

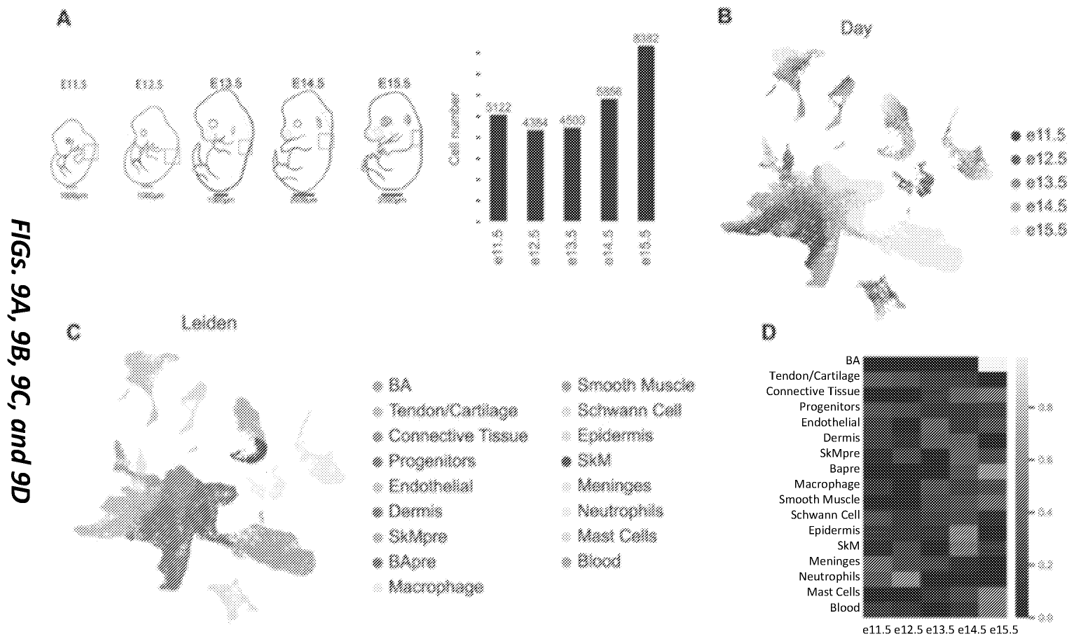

In mice, interscapular brown adipose tissue (BAT) develops in between the dermis, trapezius muscle, pectoralis muscle, and deep dorsal muscle bundles. To characterize the developmental trajectory of the developing brown fat, we isolated dorsal tissues at the forelimb level at E11.5, E12.5, E13.5, E14.5, and E15.5 (FIG. 9A) to perform single cell RNA sequencing (scRNAseq). For stages E11.5-E14.5, the isolated dorsal tissues include epidermis, dermis, mesenchyme, interscapular brown fat, and skeletal muscle. For the E15.5 time point, to enrich for BAT precursors, we removed the epidermis and underlying dermis before dissociation. Using the inDrops workflow (Klein et al., 2015), we generated single-cell transcriptomes from at least 4000 cells per stage, resulting in a final dataset of 28244 cells after quality control filtering. Uniform Manifold Approximation and Projection (UMAP) based 2-dimensional embedding show that cells from the different time points largely segregate based on transcriptional similarity rather than with embryonic age (FIGS. 9B-D). Using Leiden clustering algorithm, we identified 17 clusters representing the somitic and non-somitic cell types that reside in the dorsal trunk region of the murine embryo (FIG. 9C). We manually annotated the cell types corresponding to the different clusters using well-established marker genes for the various lineages. Among somite derivatives, we observed clusters of cells that originate from the dermomyotome, including brown adipocytes (Cidea, Ucp1), skeletal muscle (Myog, Ttn, Actn2, Myh3) and dermal fibroblasts (Twist2, Dpt, Crabp1) (FIGS. 9E-F). In addition, we could annotate clusters of smooth muscle cells (Cnn1, Acta2), muscle connective tissue (Ngfr, Osr1, Osr2), cartilage (Cnmd, Col2a1), endothelial cells (Cdh5, Kdr) and meninges (Foxc1, Cldn11, Aldh1a2) (FIGS. 9E-F). Among non-somitic cells, we found macrophages (Csf1r, C1qb), mast cells (Srgn, Kit), neutrophils (Ngp, Lccn2), Schwann cells (Sox10, Mpz), and epidermal cells (Krt5, Krt14) (FIGS. 9E-F). In conclusion, single-cell transcriptomics analysis captured major dermomyotome derivatives including cells of the brown adipocyte lineage present in the mouse dorsal trunk during embryonic development.

### Example 2: Characterization of the Developmental Trajectory of Brown Adipocyte Precursors in the Developing Trunk

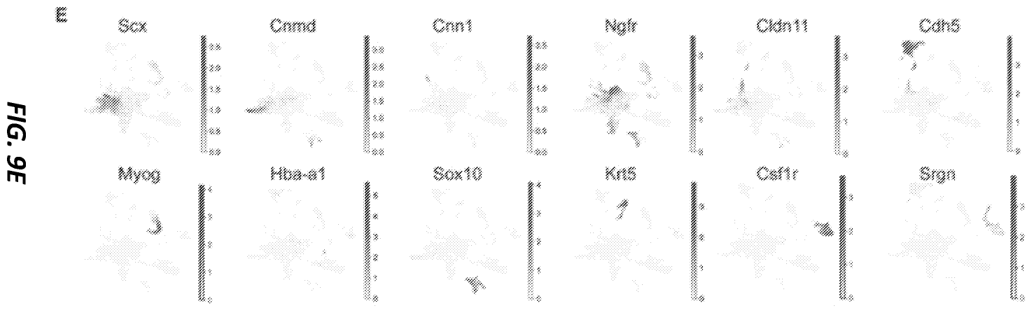

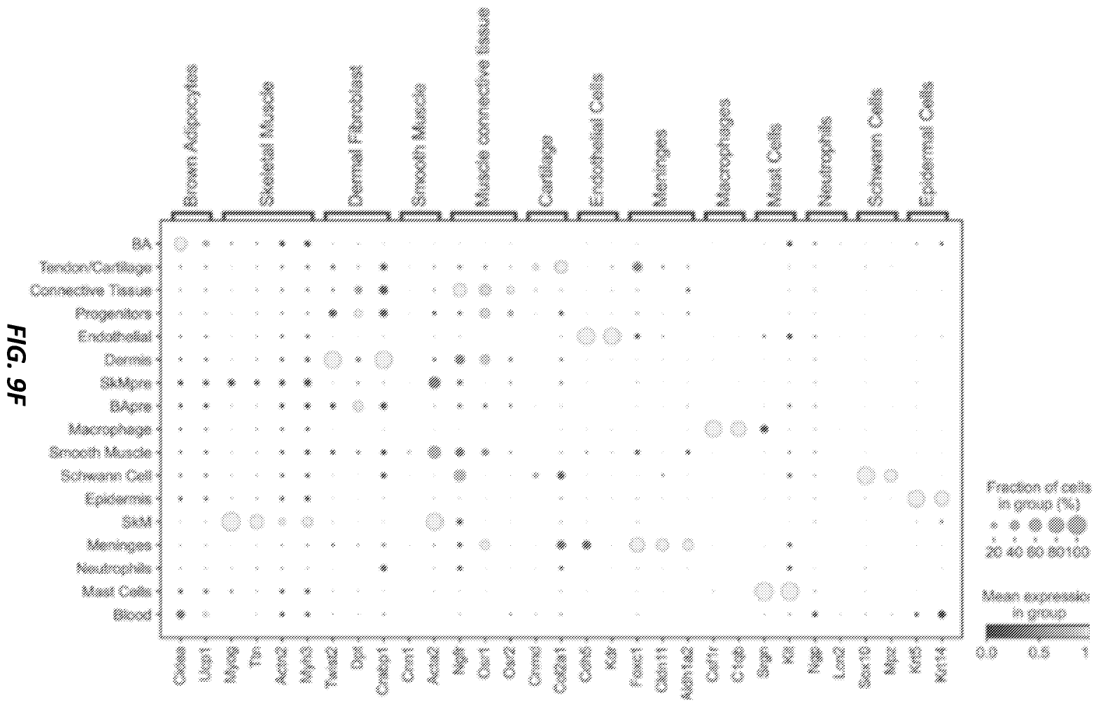

Most of the somite-derived cells, except endothelial and skeletal muscle cells, lie closely together on the UMAP embedding suggesting that they have related transcriptional signatures (FIGS. 9B-C). This large group of cells forms a single continuous ensemble including clusters corresponding to cartilage, bone, smooth muscle, connective tissue, dermis, and brown adipocytes. All these cell types arise from fibroblastic precursors thus potentially explaining the similarity of their transcriptomes. We extracted the clusters representing this group of cells from our previous analysis and reanalyzed this new dataset in detail. Most of the cells belong to a large central cluster already present at E1 1.5, which we name Fibroblastic Progenitors (FP) (FIGS. 1A-B). Cells of this cluster expressed markers classically associated to fibroblasts including Pdgfra or Hic (FIG. 1C). Differential gene expression analysis of the clusters showed that the differentially expressed genes of the FP cluster do not contain characteristic lineage markers. Moreover, the FP cluster connects to all the different fates, suggesting that it corresponds to a multipotent progenitor population. The FP cluster is linked to a cluster of prospective brown adipocytes, which we termed Brown Adipocyte Precursors (BAPre) (FIG. 1A). These cells expressed brown adipocyte precursor markers such as Ebf2, Cebpa and Pparg (FIG. 1C, FIG. 10A). The BAPre cluster is connected to a cluster of brown adipocytes characterized by the expression of Cidea or Ucp1 (Wang et al., 2014), FIG. 1C, FIG. 10A). The BAPre cluster was first detected at day 13.5 and its size progressively increases until E15.5, while the brown adipocyte cluster is first detected at E15.5 (FIG. 1D). Pseudotime analysis supported the existence of a developmental trajectory for the brown adipocyte lineage which starts from FP cells followed by BAPre and finally brown adipocytes (FIG. 1F). To further characterize the developmental trajectories arising from the FP cluster, we performed an analysis with the STREAM pipeline (Chen et al., 2019), which confirmed these putative lineage relationships among clusters (FIG. 1E). These conclusions are further supported by an analysis using the Waddington OT pipeline that characterized the ancestor/descendant sequence based on cell-to-cell transitions probabilities and gene expression similarities (Schiebinger et al., 2019) (FIGS. 10B-C). These analyses demonstrate that cartilage, bone, tendon, connective tissue, dermis, and brown adipocytes all arise from the FP cluster and that the brown adipocyte cluster derives from the BAPre cluster (FIGS. 1E-F).

This developmental trajectory of the brown fat lineage was confirmed by immuno-histochemistry of sections of the dorsal region of the developing mouse brachial trunk with markers of the adipocyte lineage. Immunofluorescence analysis of E12.5 to E15.5 embryos confirmed the expression of Ebf2 in the presumptive interscapular brown fat region but also in skeletal muscle and precartilage (FIG. 1G) (Wang et al., 2014). Developing adipocyte precursors expressing Pparg probably corresponding to BAPre started to appear at E13.5 and developed in between the dermis and Myosin Heavy Chain (MyHC) expressing muscle fibers (FIG. 1G). Perilipin1 (Plin1) staining showed that a small number of preadipocytes start to accumulate lipid vesicles at E14.5. An increasing number of lipid-containing immature adipocytes appeared by E15.5 (FIG. 1G).

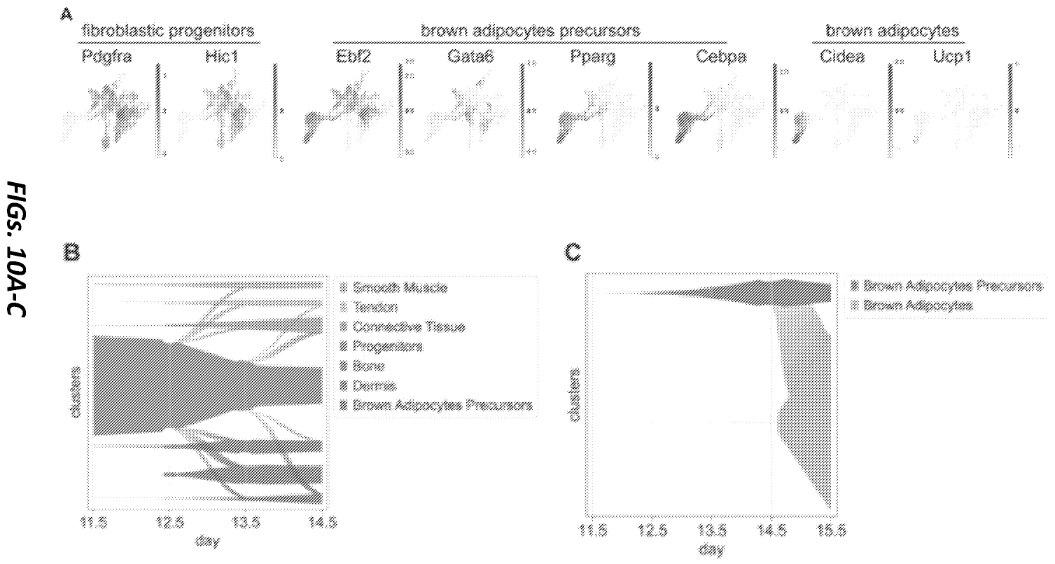

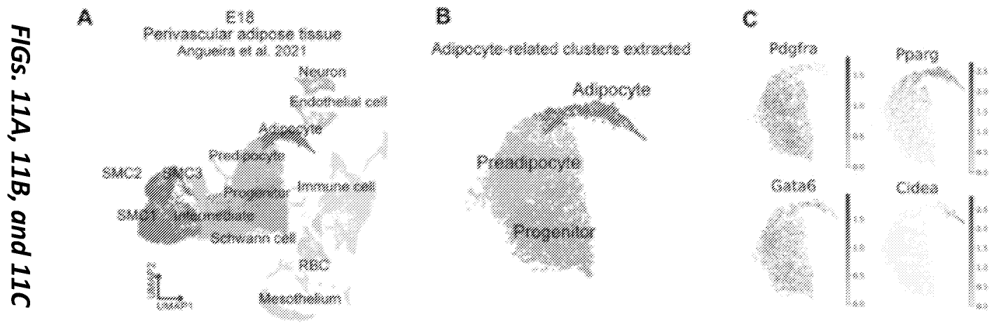

Angueira et al. (2021) recently identified progenitors that give rise to brown adipocytes in mouse periaortic (pVAT) brown adipose cells analyzed by scRNAseq at E18 and perinatal stages (Angueira et al., 2021). We reanalyzed their E18 dataset which contains 16,967 single cell transcriptomes. Following UMAP embedding and Leiden clustering, we identified 13 clusters representing the cell types described in the original study (FIG. 11A). After manual annotation of the adipocyte clusters, we identified a trajectory similar to that observed for the somitic lineages starting with a cluster of Fibroblastic Progenitors (Pdgfra) followed by BAPre (Pparg) and by brown adipocytes (Cidea) (FIGS. 11B-C). Pi16, Cd34 and Ly6a, which were identified as brown adipocyte progenitor markers in the Angueira report are also differentially expressed in the Progenitor population identified in our study although they are only detected after E13.5, suggesting that the FP population matures over time (FIG. 11D). Using a machine learning classifier trained on the Angueira dataset, we confirmed the similarity between the progenitors and brown adipocytes fates identified in the two studies. However, the preadipocyte cluster identified in their study appears to correspond to a more immature stage of the BA lineage differentiation compared to the BAPre cluster of our mouse dataset as it more closely resembles our FP cluster (FIG. 11E). Immunofluorescence analysis at E15.5 confirmed the expression of the adipocyte precursors markers Dpp4, Cd34 and Ly6a at the periphery of the developing brown adipose tissue of the interscapular depot (FIGS. 11F-G). Their location suggested that they might correspond to precursor cells involved in the generation of mature brown fat. Together, these data suggest that the brown adipocytes of the interscapular region derive from a multipotent population of fibroblastic progenitors expressing markers such as Pdgfra which gives rise to an immature population of lineage restricted precursors (BAPre) appearing around E13.5. Around E15.5, this population starts giving rise to brown adipocytes identified by specific markers such as Cidea.

### Example 3: Gata6 Marks Early Brown Adipose Tissue Precursors

The transcription factor Gata6 has been previously associated with the brown adipocyte lineage (Cheng et al., 2018; Wang et al., 2014), but its developmental expression has not been characterized. In our dataset, we observed a striking restriction of Gata6 expression to the brown adipocyte and smooth muscle lineages (FIG. 1C). Extraction of the Gata6-expressing cells from the mouse dataset shows that these cells mostly belong to these two lineages with the majority of them belonging to the FP cluster (FIG. 2A). Gata6-expressing cells are first detected in the FP, then in the BApre and in the brown adipocyte and smooth muscle clusters. Gata6 expression progressively decreased during the differentiation of brown adipocytes (FIG. 1C). These observations were confirmed at the protein level by immunofluorescence analysis, which indicated that Gata6 is detected at E12.5 in the presumptive brown fat area under the dermis (FIG. 2B). Differentiating Pparg-positive immature adipocytes as well as Pparg-negative adipocyte precursors expressed Gata6 at E14.5 (FIG. 2B). At E15.5, most Pparg-positive cells of the forming interscapular BAT had downregulated the Gata6 protein. Strong expression of Gata6 was still observed in Pparg-negative cells at the periphery of BAT, possibly corresponding to brown adipocyte precursors (FIG. 2B). Downregulation of both Gata6 mRNA and protein in brown adipocytes at E15.5 suggests that Gata6 is transiently expressed during brown adipocyte differentiation and is downregulated in more mature brown adipocytes. Similar expression of Gata6 during brown adipocyte differentiation was observed in the Angueira dataset (FIG. 11C).

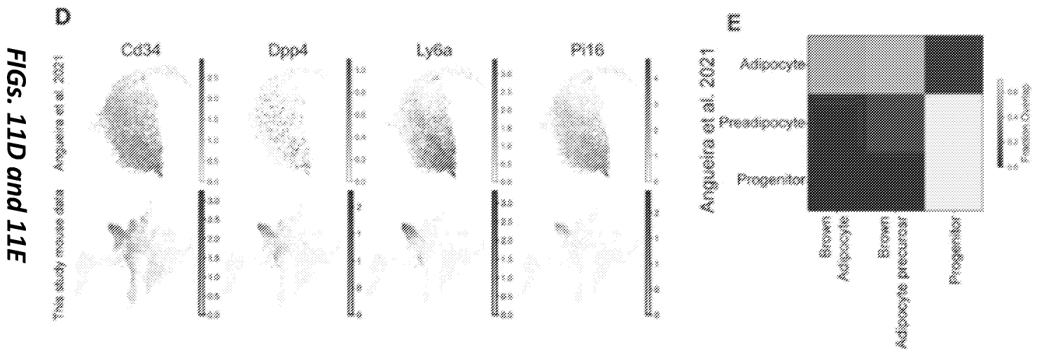

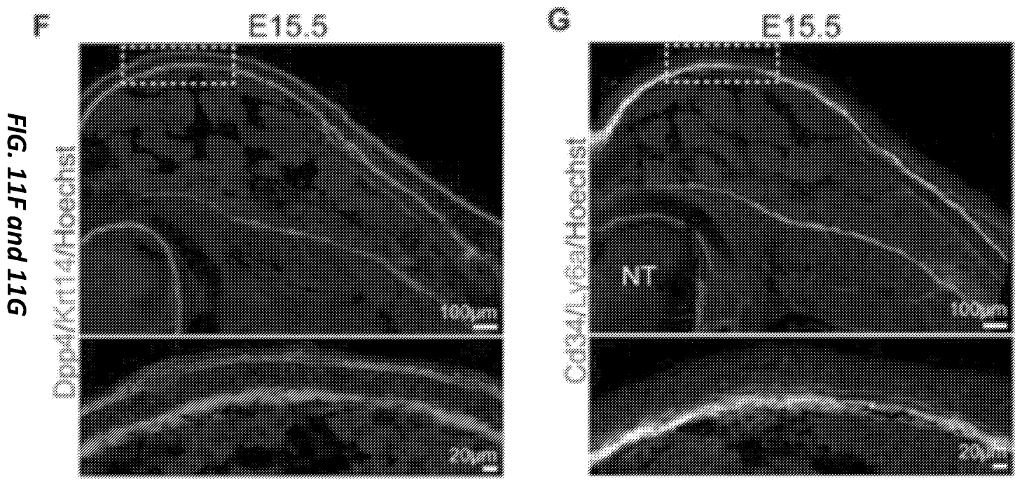

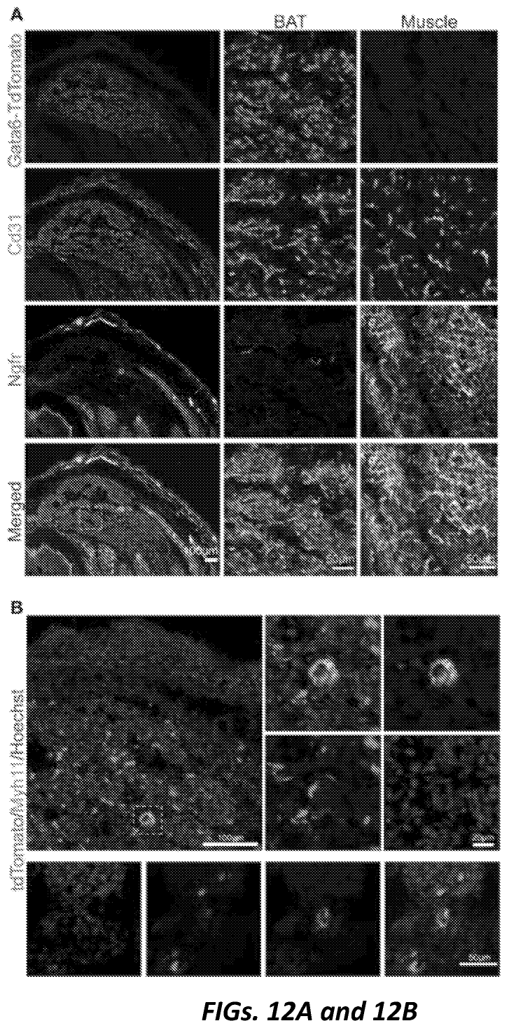

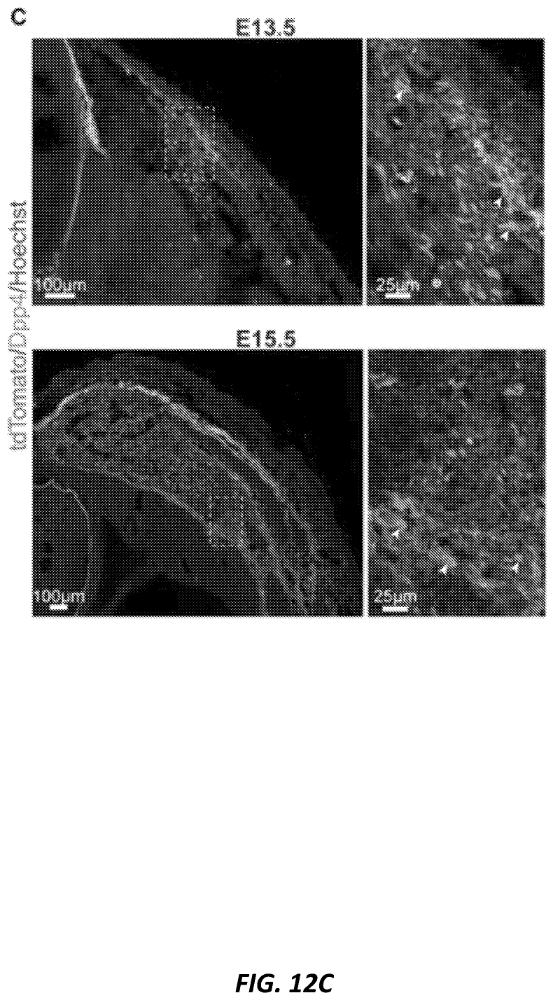

To experimentally demonstrate that Gata6 is expressed by precursors which give rise to brown adipocytes, we used a Gata6-CreERT2:Rosa26-tdTomato mouse line to label Gata6-expressing cells during embryonic development (Donati et al., 2017). We induced recombination using tamoxifen injection at E12.5 and analyzed the contribution of labelled cells to brown adipose tissue at E15.5 (FIG. 2C). Immunofluorescence analysis for tdTomato, which labels the descendants of Gata6-expressing cells confirmed that at E12.5, they give rise to heart and lung bud cells as expected (FIG. 2C). Among the somite-derived cells, tdTomato positive cells were mostly found in the interscapular BAT region and not in other somite derivatives such as skeletal muscle, cartilage, or endothelial cells (FIG. 12A). No TdTomato-positive cells were detected in skeletal muscle, and we did not observe any overlap with the CD31+ population of endothelial cells in the brown fat tissue (FIG. 2C, FIG. 12A). TdTomato-positive cells in the interscapular BAT also stained positive for Pparg at E15.5 confirming that these cells are adipocytes (FIG. 2C). We also found rare cells next to the blood vessels in the interscapular brown adipose tissue where the tdTomato expression showed some overlap with Myh11 staining (FIG. 12B). Surprisingly however, the number of such potential smooth muscle cells labelled was much lower than expected from the scRNAseq analysis. These cells could correspond to the recently identified brown adipocyte precursors deriving from the vascular smooth muscle lineage (Shamsi et al., 2021). Also, cells expressing both TdTomato and Dpp4 were observed in the periphery of the forming brown fat mass, supporting the expression of Gata6 by brown fat precursors (FIG. 12C). In conclusion, the lineage tracing analysis argues that Gata6-expressing cells present at E12.5 in the mouse dorsal trunk mostly belong to the brown adipocyte lineage.

To further investigate the origin of these Gata6 precursors, we used a Pax7-Cre:Rosa26-mTmG mouse line to analyze the contribution of Pax7 descendants to Gata6-positive brown fat precursors at E14.5 (Hutcheson et al., 2009). As expected, dorsal, ventral and limb skeletal muscle as well as Pparg-expressing prospective brown adipocytes precursors were identified as Pax7 descendants (FIG. 2D). Membrane GFP also labeled Gata6-positive cells in the brown fat region indicating that these cells derive from an earlier Pax7-expressing population (FIG. 2E). Taken together, our experiments indicate that Pax7-positive somitic progenitors give rise to a Gata6-positive precursor population that differentiates into brown adipocytes.

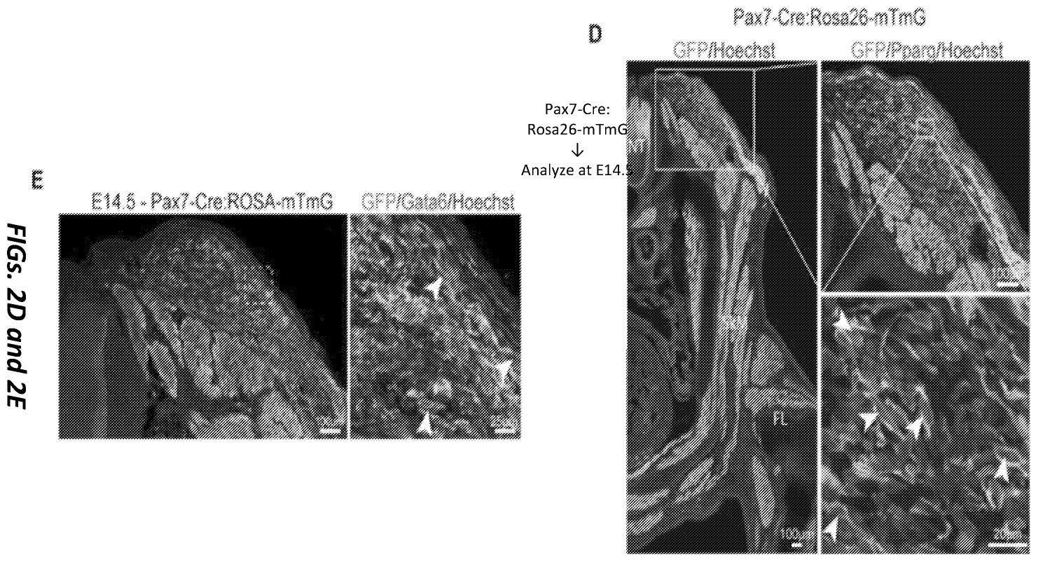

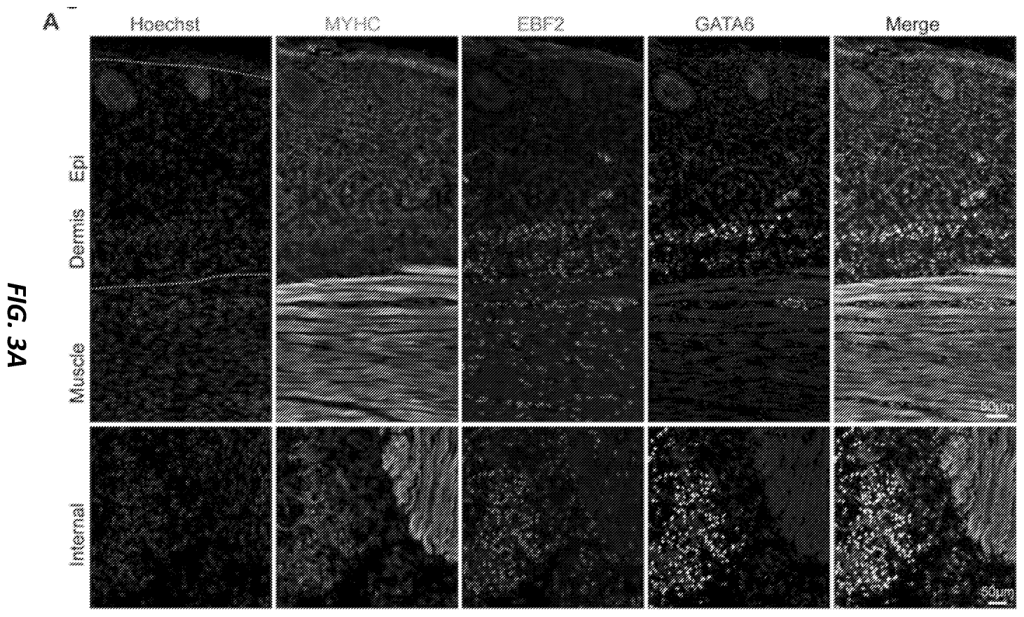

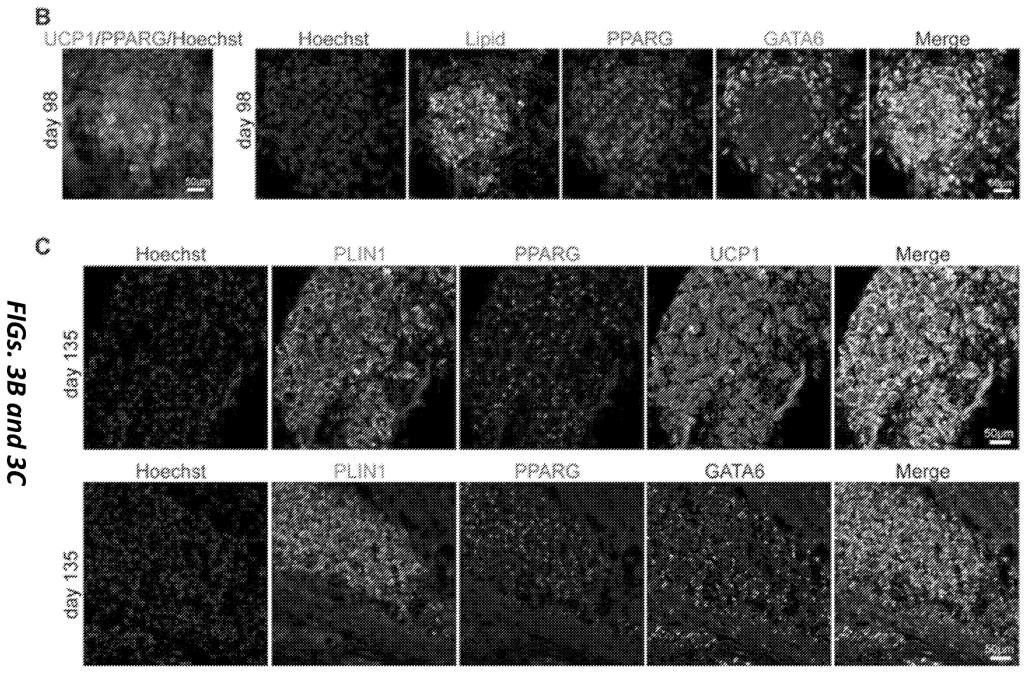

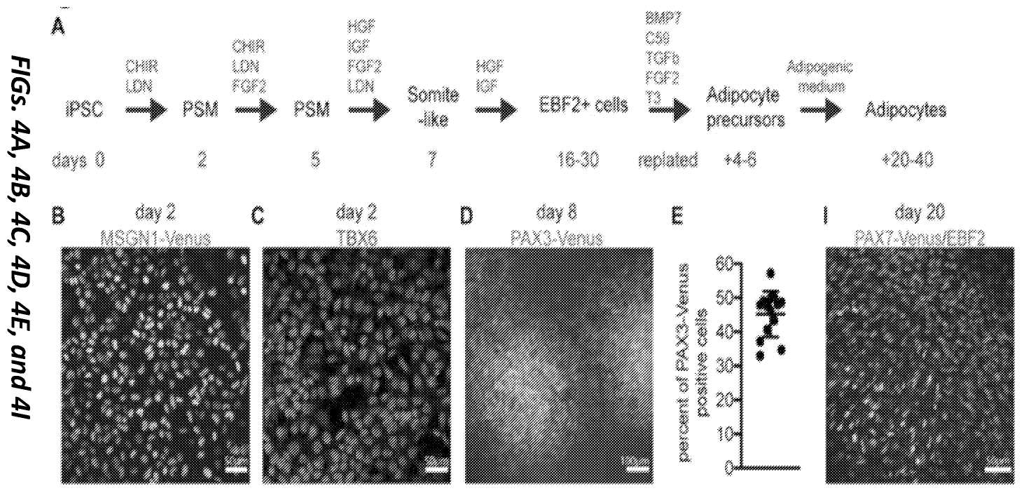

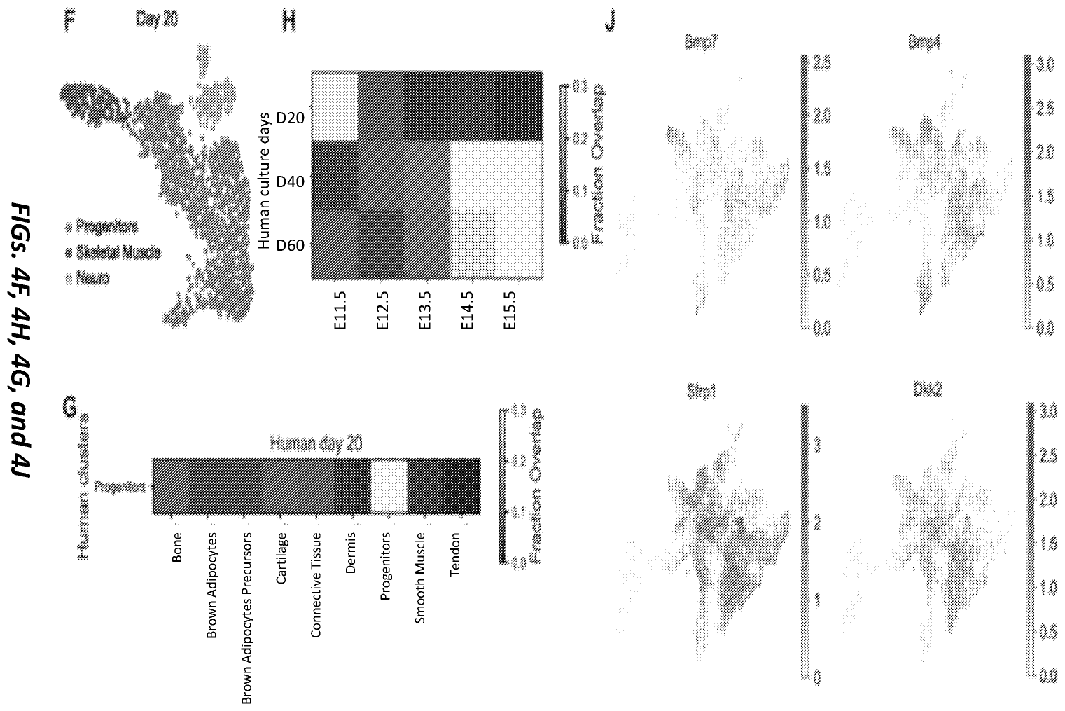

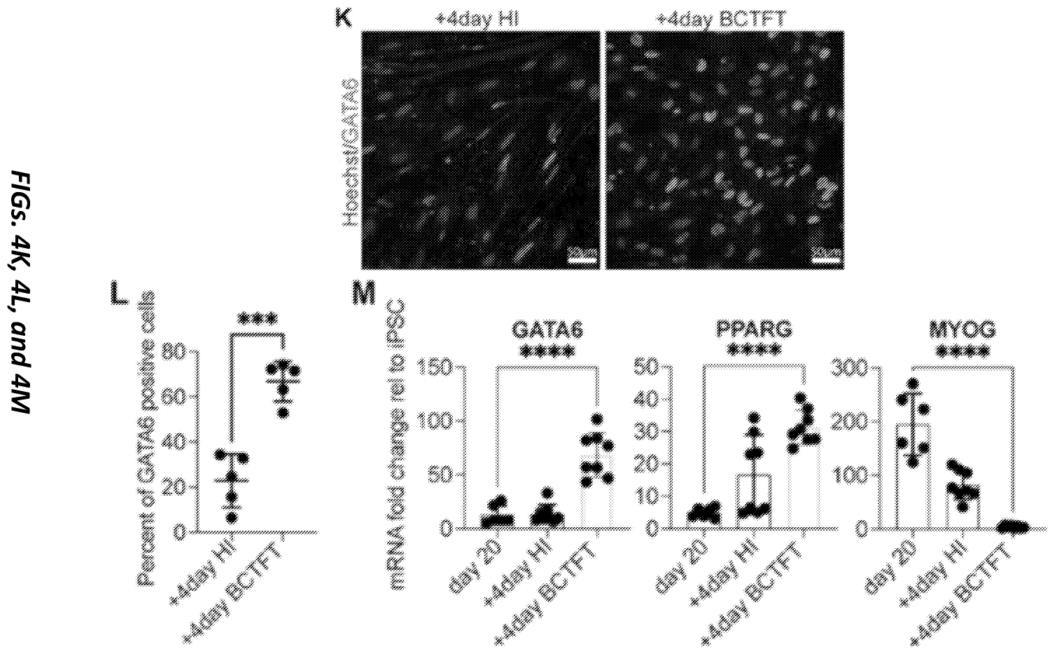

To further examine whether Gata6 expression is conserved in human brown adipogenesis, we performed immunofluorescence analysis on 98- and 135-day-old (estimated post-conceptual age) human fetal tissue from the interscapular and scapular regions. We detected expression of EBF2 and GATA6 in cells adjacent to skeletal muscle tissue (detected by MYHC antibody) under the dorsal dermis and dispersed between the deeper dorsal muscle bundles at 98 days of development (FIG. 3A). As in mouse, EBF2 was expressed in a broader territory in the dermis and deeper muscle areas. We also observed a few lipid-filled UCP1- and PPARG-positive adipocytes in the deeper muscle area (FIGS. 3B-C). In these regions, we detected PPARG and GATA6 double-positive cells, supporting the existence of GATA6-positive brown adipocyte progenitors in humans (FIGS. 3B-C). More mature brown adipocytes identified by the presence of lipid droplets, expression of PLIN1 and high expression of PPARG did not express GATA6 suggesting that GATA6 expression is also restricted to an early stage of brown adipocyte development in humans.

### Example 4: Recapitulation of Human Brown Fat Development In Vitro from Pluripotent Stem Cells

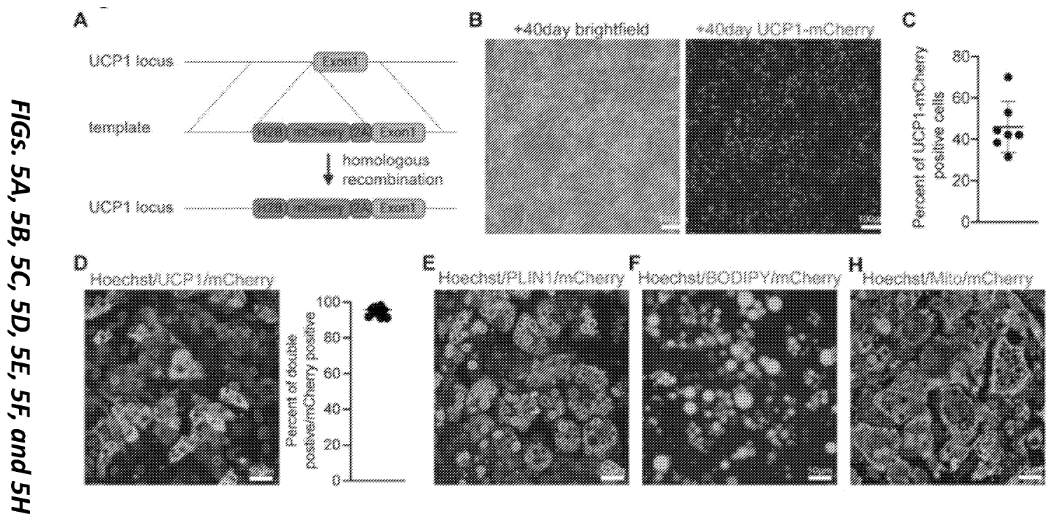

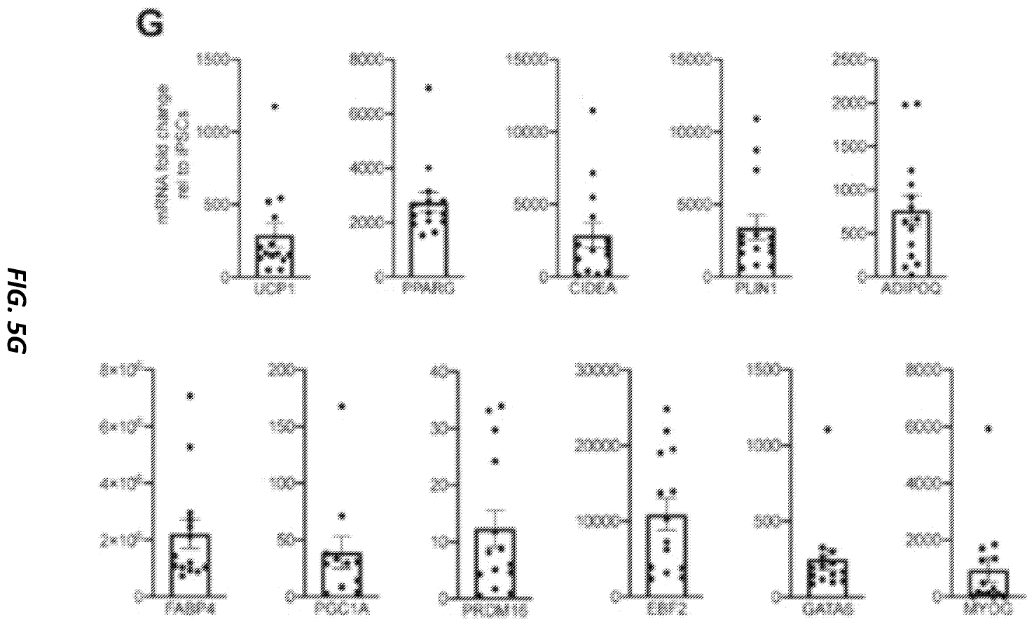

We next decided to take advantage of our characterization of mouse brown fat development to inform the design of a differentiation protocol aiming to recapitulate this process from human pluripotent stem cells. We previously developed efficient protocols to differentiate human paraxial mesoderm and skeletal muscle from iPS cells in vitro (Chal et al., 2016; Chal et al., 2015; Diaz-Cuadros et al., 2020). As brown adipocytes originate mostly from the paraxial mesoderm and share a common precursor with skeletal muscles in the dermomyotome, we first aimed to recapitulate the development of Pax-3 positive dermomyotomal cells in vitro. To achieve this, we first treated human iPSCs with the Wnt agonist CHIR and the BMP inhibitor LDN in serum-free conditions to differentiate them to presomitic mesoderm (PSM) (FIG. 4A). At day 2-3, most of the cells expressed the presomitic mesoderm (PSM) markers MSGN1 (95±0.4%) and TBX6 (FIGS. 4B-C, FIG. 13A) indicating that they acquired a PSM fate. To further differentiate the PSM cells into dermomyotomal cells, we treated cultures with IGF, FGF, HGF, and LDN (Chal et al., 2016; Chal et al., 2015). To monitor cell differentiation towards the dermomyotome fate, we used CRISPR-Cas9 to knock-in a Venus fluorescent reporter into the PAX3 locus in the NCRM1 iPSC line (FIG. 13B). We used this reporter line to track the appearance of PAX3 in differentiating cells in vitro. On day 8, differentiating cells started to express PAX3, indicating the appearance of somitic/dermomyotomal cells (FIG. 4D). 45±6.7% of the cells in the culture were PAX3-positive at this stage (FIG. 4E, FIG. 13C). We further differentiated these cultures in medium containing HGF and IGF as this treatment promotes the differentiation of somitic PAX7 precursor cells (Chal et al., 2016; Chal et al., 2015). In these cultures, PAX7 appeared around day 14 and at day 20 around 20-30% of the mononucleated population expressed PAX7 (FIG. 4I) (Al Tanoury et al., 2020). To investigate the cell composition of such cultures, we performed scRNAseq analysis at day 20. We analyzed 1671 cells and observed a major cluster containing cells expressing fibroblastic markers such as PDGFRA, HIC1 and EBF2 (FIG. 4F, FIG. 13D). This cluster also contained a subpopulation of PAX7-positive cells which was connected to a smaller cluster corresponding to differentiating myogenic cells (MYOD1, MYH3) (FIG. 13D). A small cluster of neural cells expressing SOX2 was also observed at this stage. Using a machine-learning classifier trained on the mouse dataset, we observed that the identity of this human progenitor cluster is related to the mouse FP cluster (FIG. 4G). Interestingly, using the classifier trained on the mouse embryonic time points, we found that these d20 human cultures were more closely related to the mouse E11.5 cells (FIG. 4H). In mouse embryos, Pax7-expressing cells are able to give rise to both skeletal muscle and brown fat until E1 1.5 after which time they only contribute to the myogenic lineage (Lepper and Fan, 2010). In the d20 cultures, most of the cells of the FP cluster expressing EBF2 were negative for PAX7 at the mRNA and protein levels suggesting that the two populations were already separated or in the process of segregating.

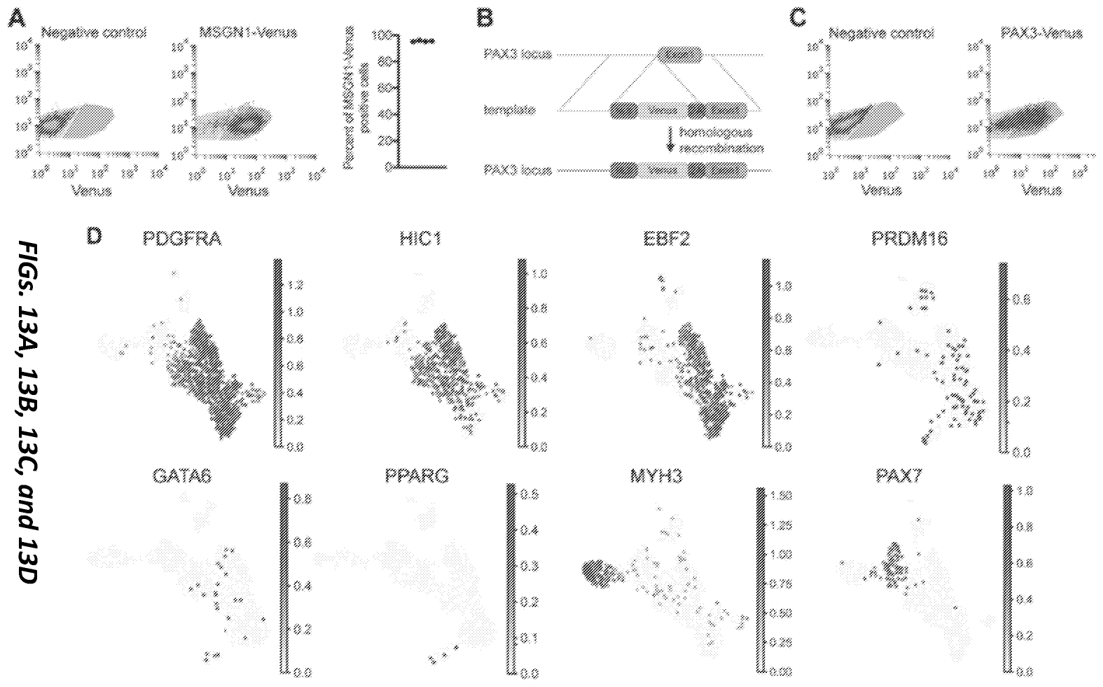

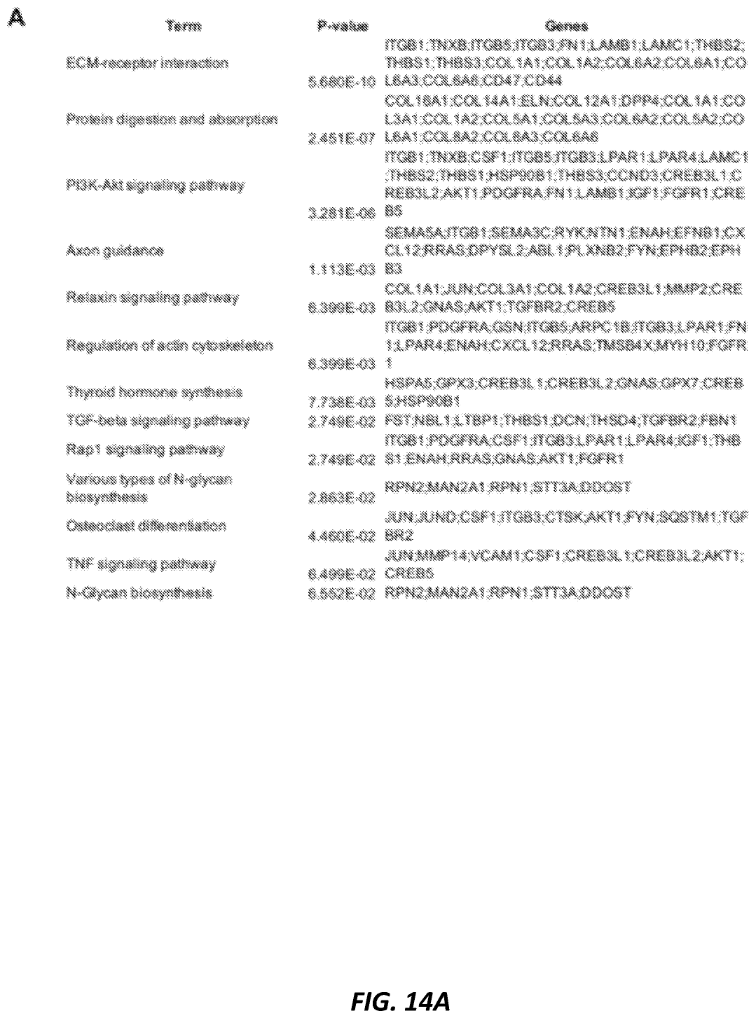

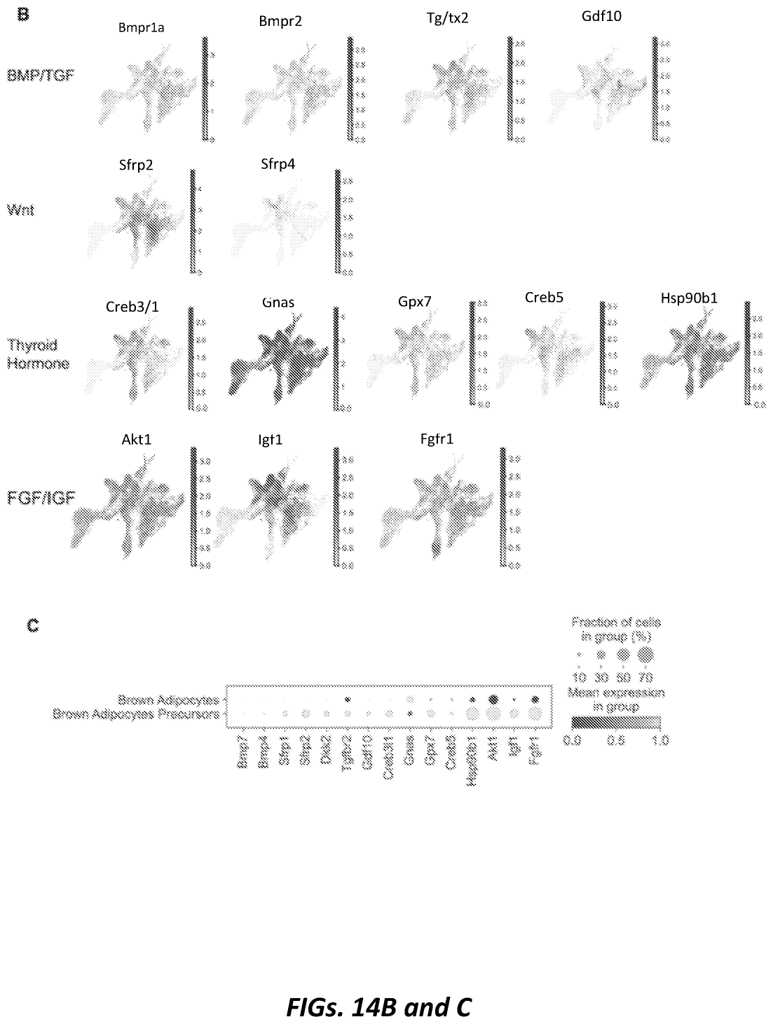

To promote the brown adipogenic fate in these cultures, we next sought to identify potential signaling pathways involved in brown adipogenesis from the mouse dataset. Mouse knock-out experiments have identified BMP activation and Wnt inhibition as important signals required for brown adipocyte development (Atit et al., 2006; Longo et al., 2004; Tseng et al., 2008). However, these studies have not precisely defined the time window when these cues are present during development. We observed expression of the BMP ligands (Bmp7, Bmp4) and of the Wnt signaling antagonists (Sfrp1, Sfrp2, Sfrp4, Dkk2) mostly at the FP stage in the mouse dataset (FIG. 4J, FIG. 14). Expression of these secreted cues was downregulated in more mature cells of the brown adipocyte lineage. These data suggest that BMP activation and Wnt inhibition are prominently required at the FP stage. We also performed differential gene expression analysis of the mouse dorsal trunk scRNAseq dataset. Using KEGG pathway analysis, we identified several pathway-specific genes differentially expressed in the FP cluster (FIG. 14A). These include genes involved in TGFbeta signaling (Tgfbr2, Gdf10), Thyroid hormone signaling (Creb3l1, Gnas, Gpx7, Creb5, Hsp90b1) and FGF/PI3K-AKT pathway (Akt1, Igf1, Fgfr1) (FIGS. 14A-C).

Thus, to recapitulate the sequence of signaling cues to which brown adipocyte precursors are exposed during their differentiation in vivo, we dissociated the primary cultures around day 20 of differentiation to eliminate the myofibers and replated the cells in a medium containing BMP7, the Wnt antagonist C-59, TGFb1, FGF2 and Triiodothyronine (BCTFT medium) for 4-6 days (FIG. 4A). Strikingly, after culturing cells in the BCTFT medium we saw a strong increase in number of GATA6-expressing cells compared to cells cultured in medium containing HGF and IGF (FIGS. 4K-L). The cells also showed an upregulation of GATA6 and PPARG and a downregulation of MYOG mRNA, suggesting that the BCTFT medium can promote the adipogenic fate while repressing the myogenic fate (FIG. 4M).

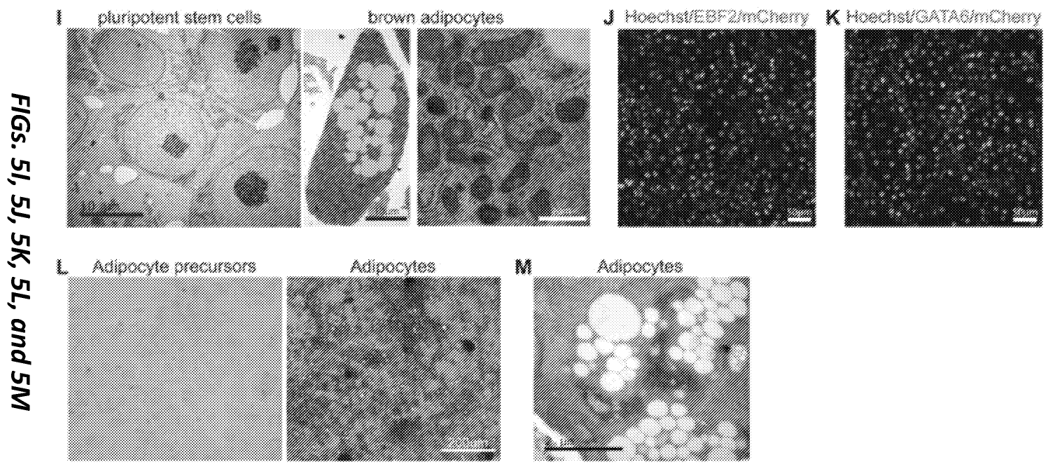

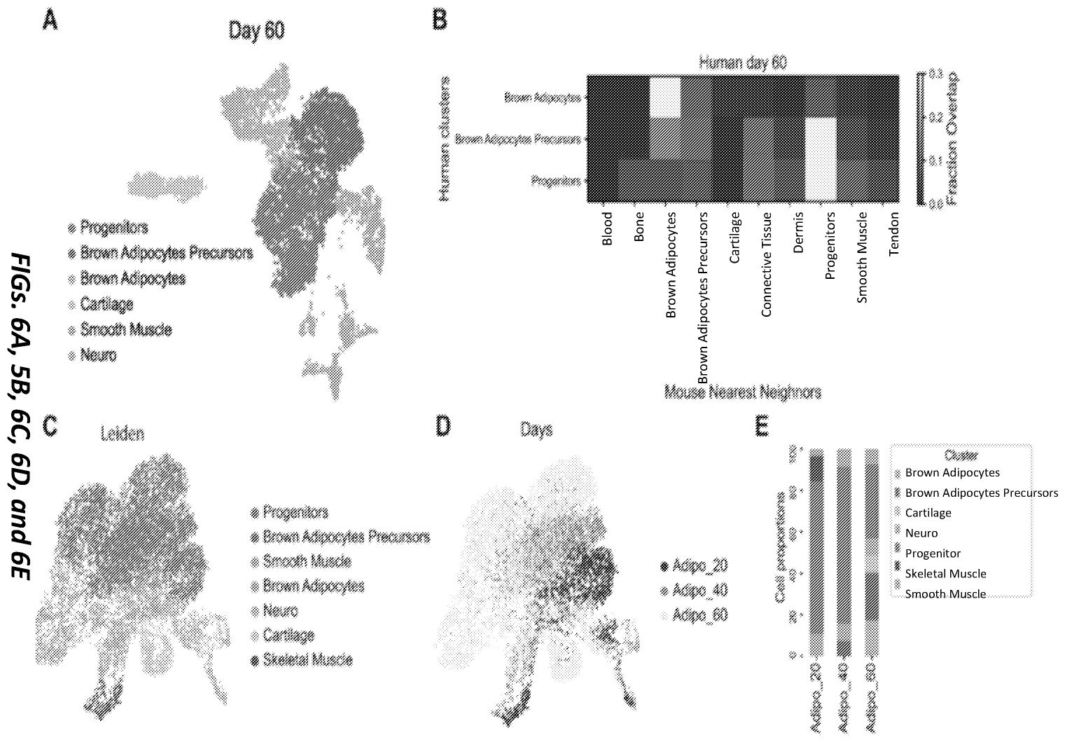

To follow the differentiation of iPSCs into brown adipocytes, we generated a UCP1-mCherry fluorescent human iPSC reporter line using CRISPR-Cas9. In this knock-in line, an mCherry construct was introduced immediately 5′ to the start codon of UCP1, from which it was separated by a P2A peptide. Thus, the reporter is produced from the same transcript as UCP1 and targeted to the nucleus using an H2B tag (FIG. 5A). The recombined locus in the reporter iPS line was sequenced to verify correct integration. Using this reporter line, we further differentiated the GATA6 positive progenitors derived in BCTFT medium. We treated the progenitor cultures with an adipogenic cocktail containing 3-Isobutyl-1-methylxanthine, Ascorbic acid, Triiodothyronine, TGFb inhibitor SB431542, Dexamethasone, EGF, Hydrocortisone and Rosiglitazone for another 20-40 days (FIG. 4A) (Hafner et al., 2016). In cultures examined after 40 days in differentiation, large UCP1-positive lipid-filled adipocytes started to appear, and the adipocyte number steadily increased over the following 20 days. We counted the number of UCP1-mCherry positive cells on day 60 of differentiation to quantify the number of UCP1-containing mature adipocytes. The cultures contained 46±12% UCP1-mCherry positive cells (FIGS. 5B-C). Using immunohistochemistry, we showed that 95% of the mCherry positive cells expressed UCP1 (FIG. 5D). PLIN1 and BODIPY staining confirmed that iPSC-derived brown adipocytes (iPSC-BAs) contain multilocular lipid droplets (FIGS. 5E-F). We further confirmed expression of brown adipocyte markers such as UCP1, PPARG, CIDEA, and PLIN1 after 60 days of differentiation using RT-qPCR in cultures (FIG. 5G).

iPSC-BAs contain abundant mitochondria (a characteristic of brown adipocytes) as revealed by immunofluorescence for mitochondrially encoded cytochrome c oxidase II (MT-C02) and electron microscopy analysis at 60 days of differentiation (FIGS. 5H-I). These cultures contained a fraction of UCP1-negative cells, which expressed EBF2 and/or GATA6 cells suggesting these cells might correspond to brown adipocyte precursor cells (FIGS. 5J-K). Periodic acid staining and electron microscopy showed that at this stage, many cells accumulate glycogen, a characteristic of differentiating brown adipocytes (FIGS. 5L-M) (Mayeuf-Louchart et al., 2019). Thus, our protocol recapitulates a developmental trajectory similar to that observed for brown fat differentiation in mouse embryos, leading to efficient differentiation of human iPSCs into UCP1-expressing brown adipocytes under serum free culture conditions.

### Example 5: scRNAseq Analysis of the Human Brown Adipogenic Lineage Differentiated In Vitro

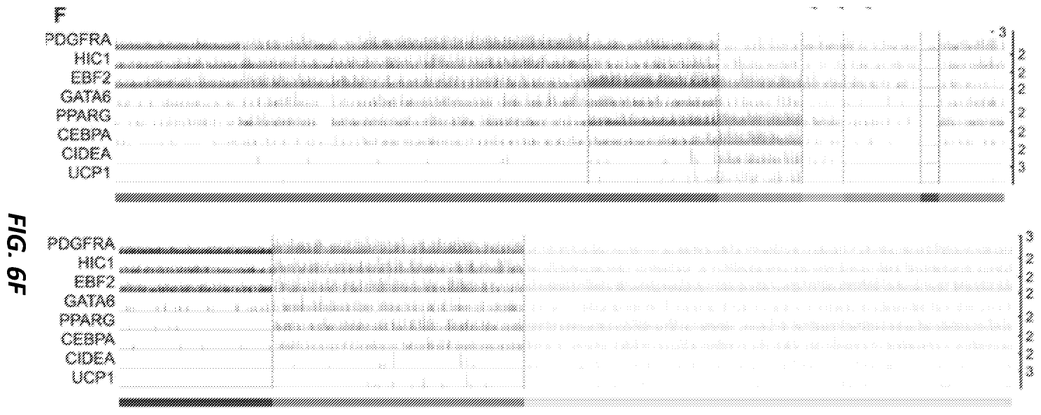

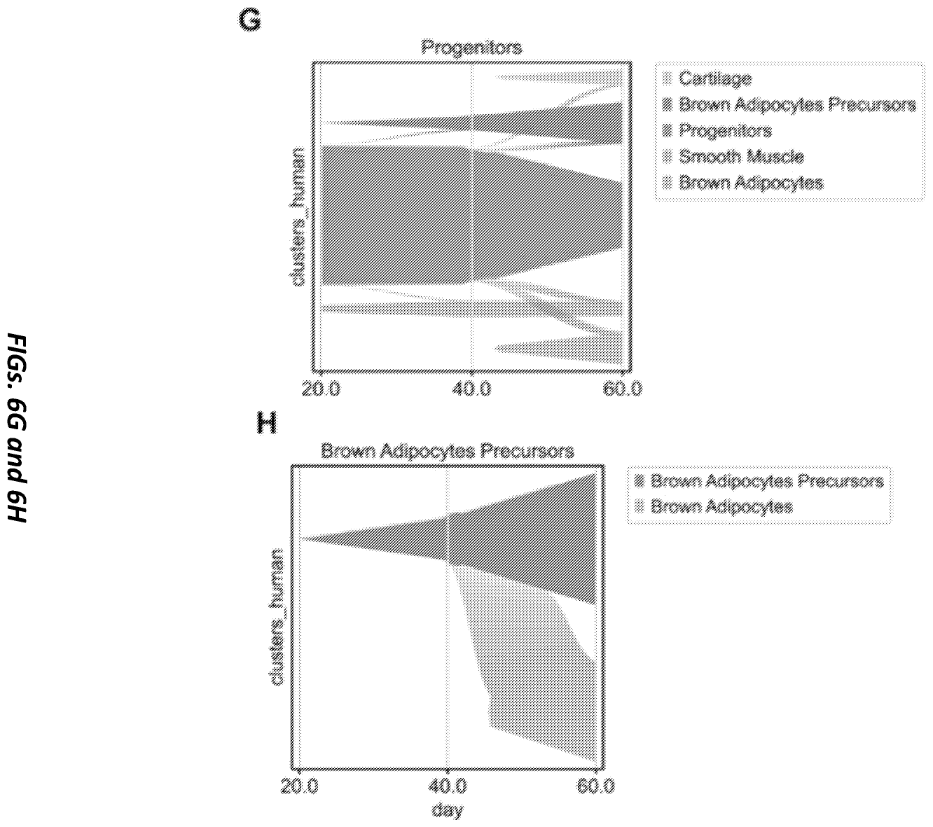

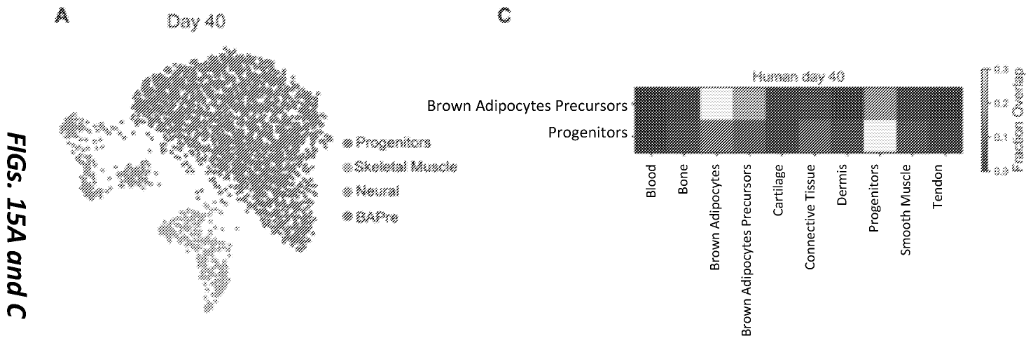

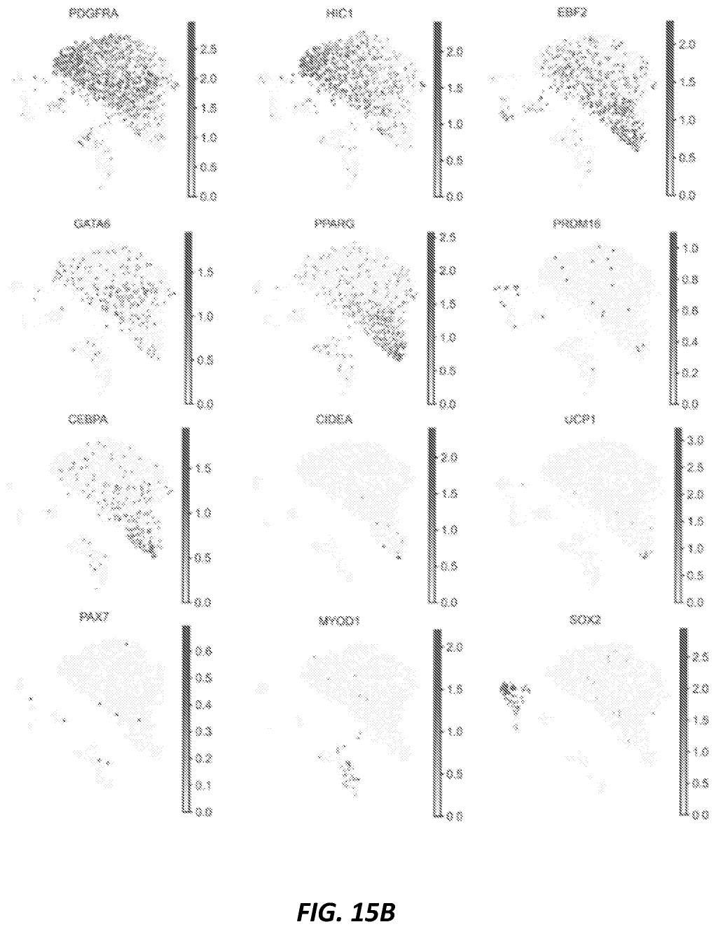

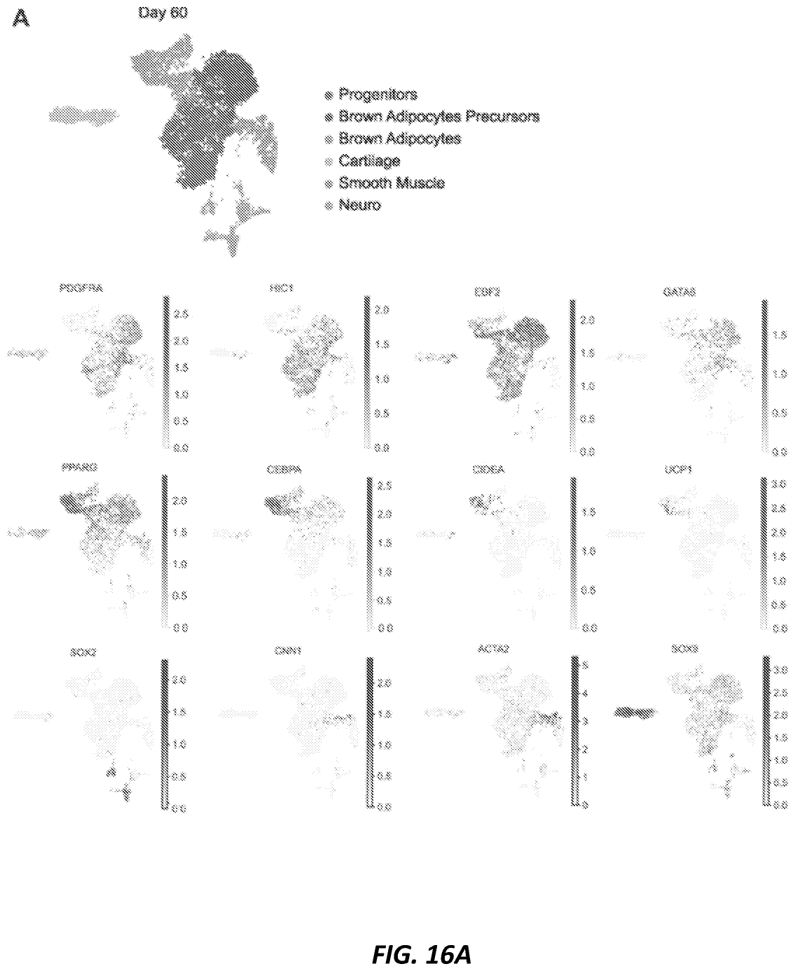

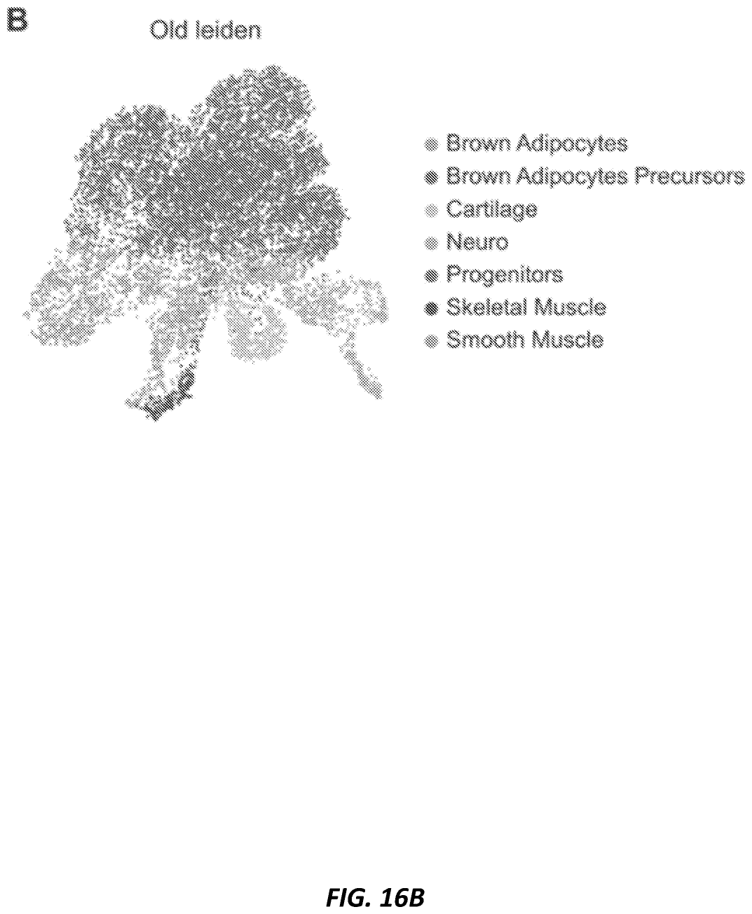

To further characterize the differentiation of iPSCs into adipocytes, we performed scRNAseq of the cultures at 40 days (d40) and 60 days (d60) of differentiation. After quality control and filtering, we analyzed 2744 and 5322 cells from d40 and d60 samples, respectively. UMAP projection followed by Leiden clustering analysis shows that at d40, most of the cells belong to a large cluster which expresses Fibroblastic markers such as PDGFRA and HIC1 (FIGS. 15A-B). Using the machine learning classifier trained on the mouse dataset, we confirmed that this cluster, which we term progenitors, is most similar to the mouse FP cluster (FIG. 15C). We also detected a cluster of cells expressing BAPre markers (EBF2, CEBPA, PPARG) which is most similar to brown adipocyte precursors and brown adipocytes clusters according to the classifier. As observed in the mouse dataset, these two clusters also show expression of GATA6. We also observed a small cluster of skeletal muscle cells (MYOD1) and one of neural cells (SOX2). At this stage PAX7 expression was undetectable. At d60, we still observed a large fibroblastic cluster expressing markers such as PDGFRA and HIC1 (FIGS. 6A-B). Comparison to the mouse dataset with the classifier shows that it is closest to the FP cluster (FIG. 6C). This cluster is connected to a cluster which expresses GATA6, EBF2 and PPARG suggesting that it resembles the mouse BAPre cluster. While the classifier analysis shows that it is more similar to the FP cluster, it also shows similarity with the mouse brown adipocyte precursors and brown adipocyte clusters suggesting that it corresponds to immature cells of the brown adipocyte lineage. We also identified a cluster of brown adipocytes (CEBPA, CIDEA, UCP1) which is most similar to the mouse brown adipocyte cluster based on the classifier analysis (FIG. 6B). Small separate clusters of cartilage (SOX9), smooth muscle (CNN1, ACTA2) and neural cells (SOX2) were also observed (FIG. 16A).

We next merged the three time points (d20, d40, and d60) to generate a UMAP projection and performed a Leiden clustering analysis on this combined dataset (FIGS. 6D-E). We found that the cell types identified in the clustering analysis of individual time points mostly merged into single clusters defined by cell identity rather than age identified in this new analysis (FIGS. 6D-E, FIG. 16B). As observed with the mouse dataset, the largest cluster corresponds to the Progenitors cluster which is connected to clusters of more mature fates. This suggests that the Progenitors cluster generated in vitro represents a population of multipotent precursors similar to the mouse FP cluster. This cluster is already present at d20 and maintained during later stages (FIG. 6F), like the FP cluster of the mouse dataset. Waddington OT analysis of the developmental trajectories of the cells differentiating in vitro suggests that the progenitors cluster contributes to the brown adipocyte precursors and smooth muscle clusters observed at d40 and d60 as well as to the cartilage identified at d60 (FIG. 6G). It also shows that the d60 brown adipocyte cluster likely derives from the brown adipocyte precursor cluster (FIG. 611).

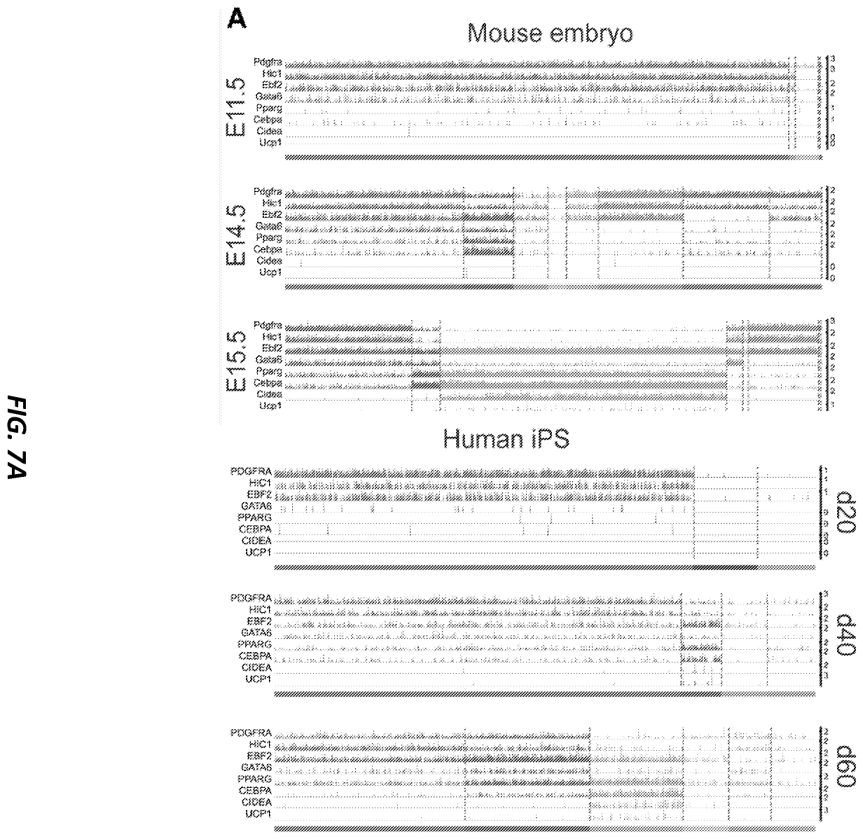

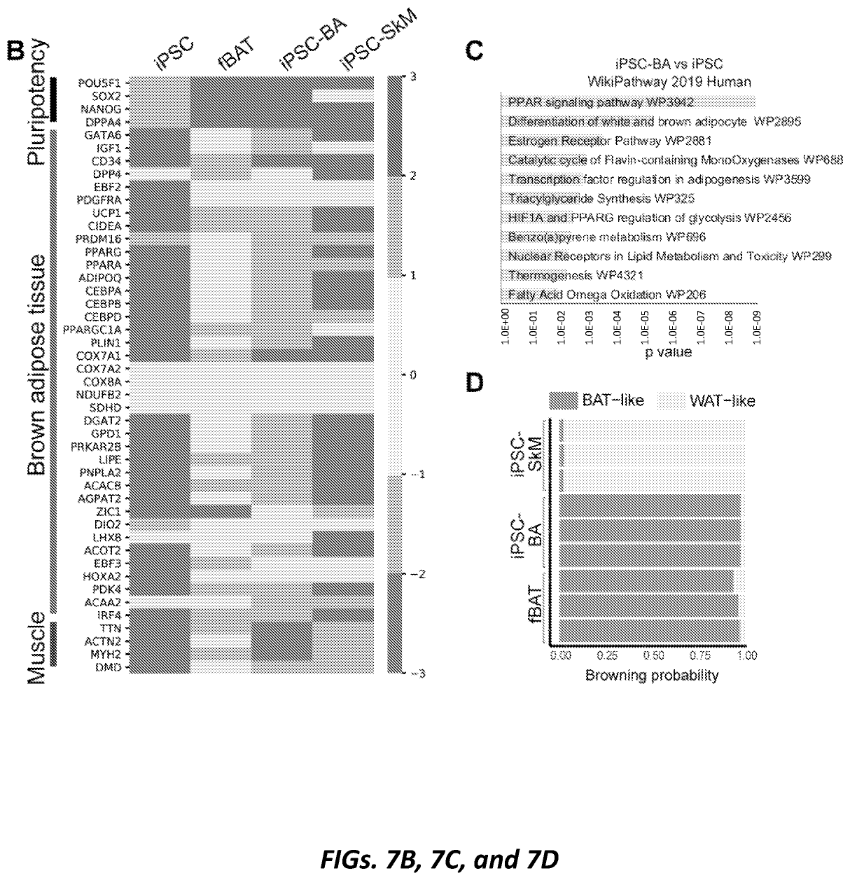

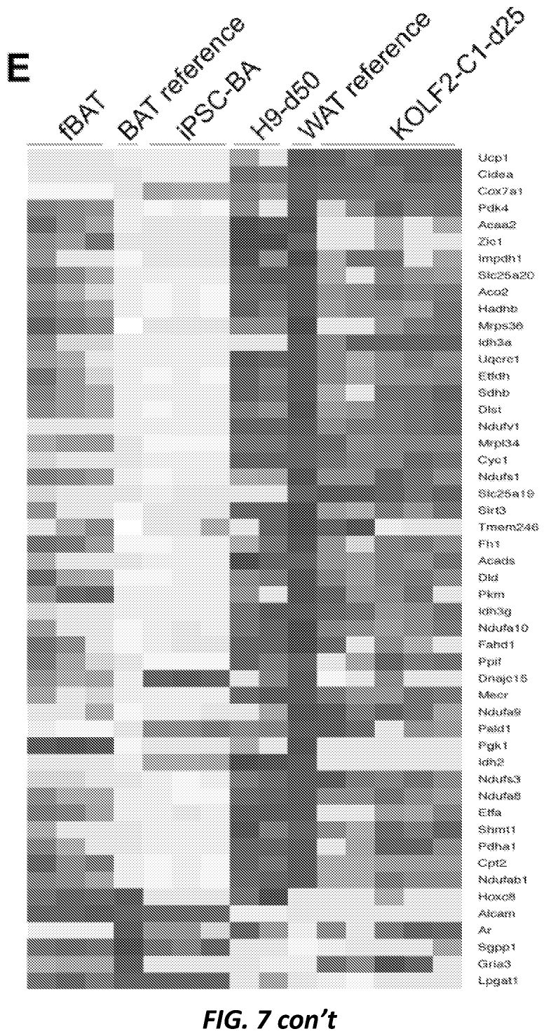

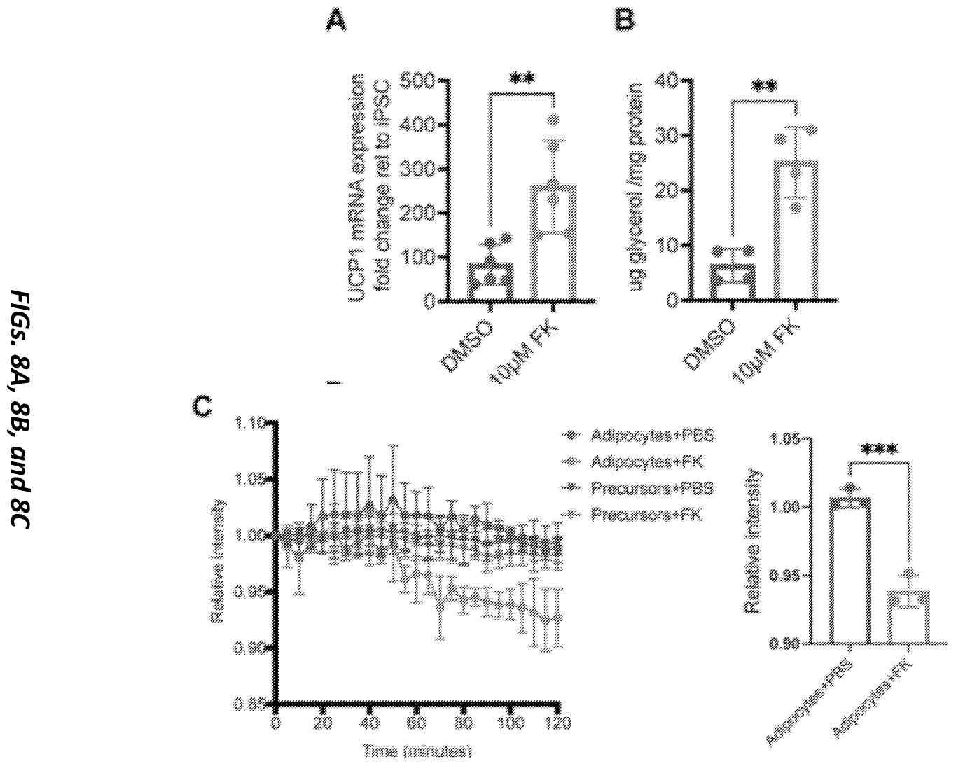

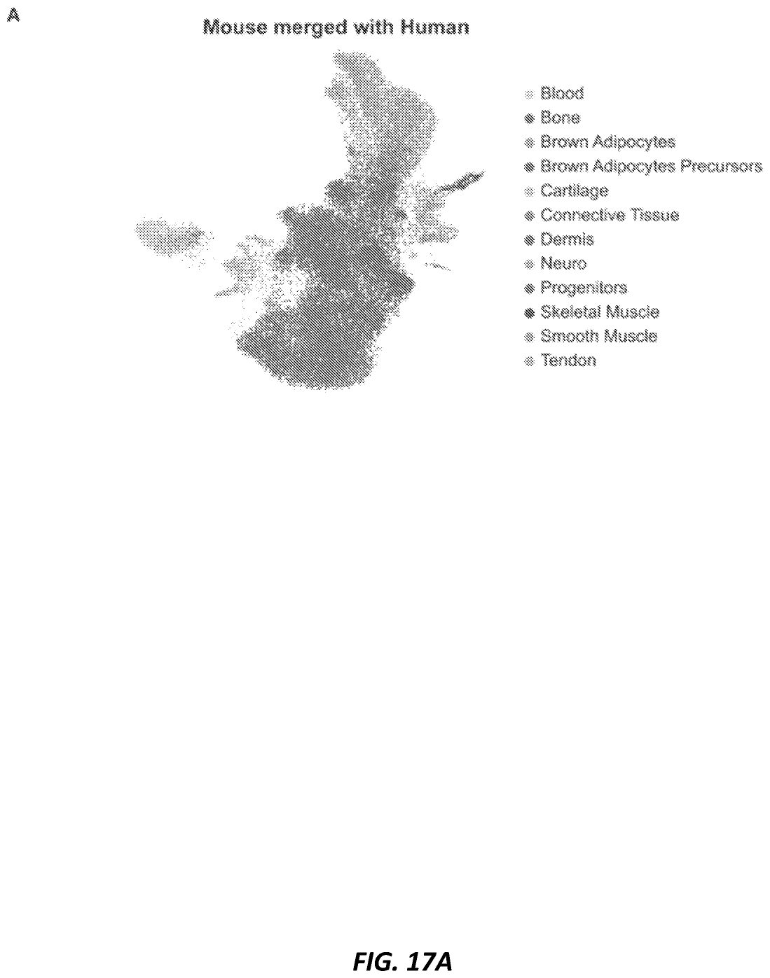

To further establish the identity of cells differentiated in vitro, we directly compared the d60 human cultures to the mouse scRNaseq dataset. The list of expressed mouse genes was converted into their human orthologs and the two datasets were merged and analyzed together using UMAP projection followed by Leiden clustering. Human and mouse cells identified as Fibroblastic Progenitors, BAPre and Brown adipocytes were found in the same clusters in the merged dataset indicating the similarity of the human cells differentiated in vitro to the mouse brown adipocyte lineage differentiating in vivo (FIGS. 17A-C). Clusters of other lineages such a cartilage or smooth muscle also contained both mouse and human cells. Side by side comparison of the expression of specific markers of brown adipocyte differentiation in the same clusters in mouse and human cells shows very similar expression patterns (FIG. 7A). Finally, we used the classifier trained on the mouse dataset to compare each time point analyzed in vitro to each of the different mouse embryonic ages analyzed. This suggests that the d40 cells are closer to E14.5-15.5 and d60 to E15.5 (FIG. 4G).

The single cell dissociation required for scRNAseq disrupts the mature lipid filled brown adipocytes, which are large buoyant cells that moreover cannot be encapsulated using the microfluidics platform used for the Indrops pipeline and are thus absent from the scRNA dataset. Accordingly, we only detected ˜1% of cells expressing the UCP1 transcript in the d60 scRNAseq dataset whereas counting the UCP1-mCherry positive cells using fluorescence microscopy in d60 cultures yields an estimate of ˜40-50% positive cells. Nevertheless, our scRNAseq analysis suggests that in d60 cultures, ˜40% of the cells belong to the BAPre and brown adipocyte clusters while another 40% of the cells are in the Progenitors cluster. Therefore, an approximate 80% of the cells of the scRNAseq dataset represent different stages of the brown adipocyte lineage differentiation. Together, this suggests that the vast majority (possibly up to 80-90%) of the d60 cultures corresponds to cells at various stages of the differentiation of the brown adipocyte lineage. In conclusion, this analysis suggests that a brown adipocyte lineage developmental trajectory highly similar to that observed for mouse interscapular fat in vivo can be recapitulated in vitro from human iPS cells. This results in the production of immature brown adipocytes approximately equivalent to those of the E15.5 mouse embryo.

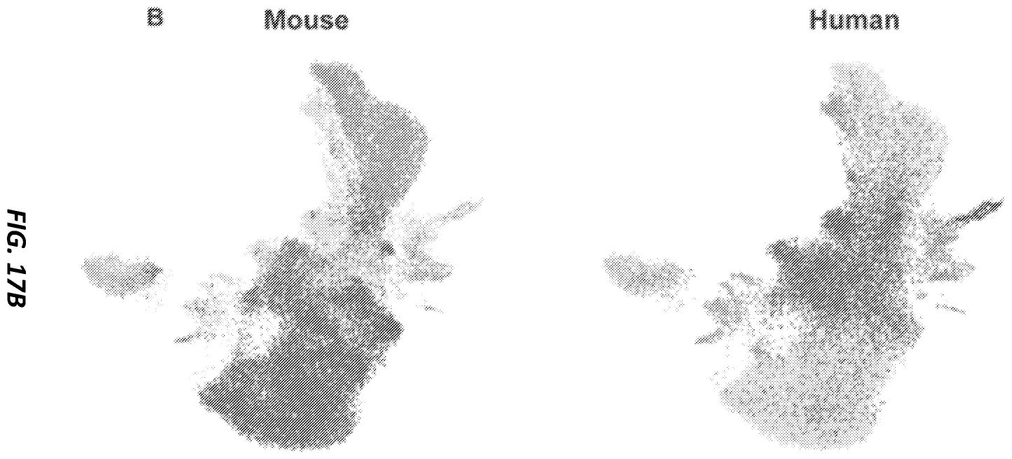

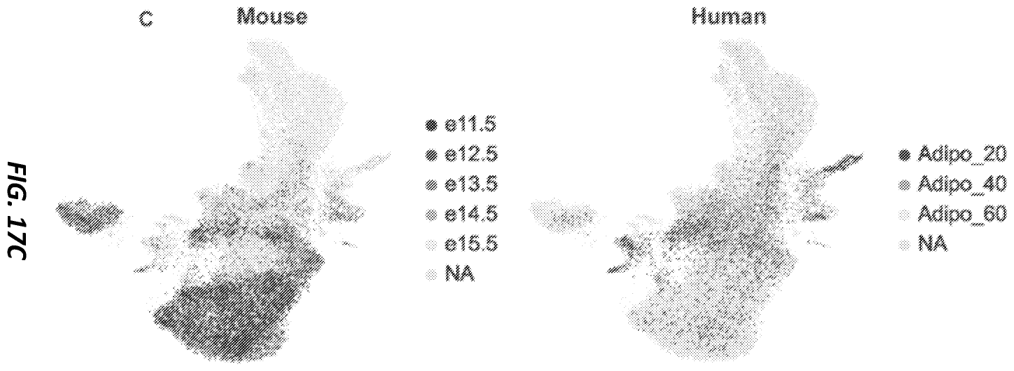

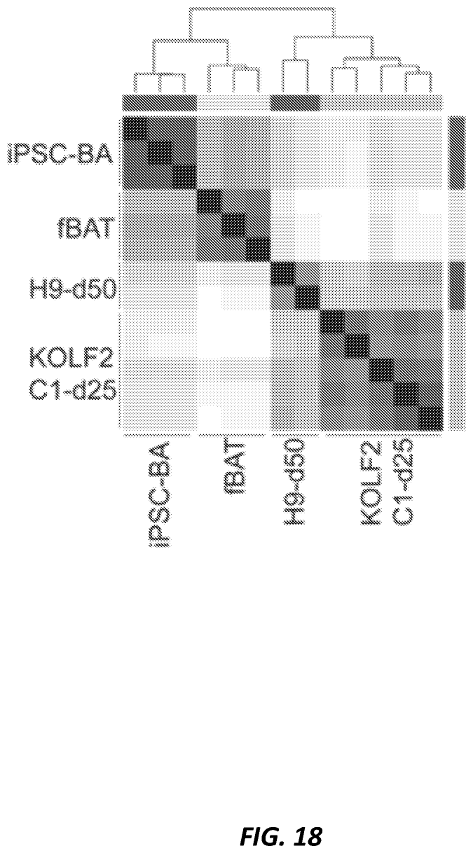

To molecularly characterize the iPSC-BAs which are mostly lost during dissociation in the scRNAseq analysis, we performed bulk mRNA sequencing of undifferentiated iPSCs and cultures at 60 days of differentiation. In parallel, as a reference for differentiated adipocytes, we analyzed human fetal BAT (fBAT) isolated from 115, 122 and 125-day post-conceptual age fetuses from the interscapular and scapular regions. As an outgroup, we used bulk RNAseq data from iPSC-derived skeletal muscle cultures (iPSC-SkM) (Al Tanoury et al., 2021). Brown adipocyte-specific genes such as UCP1, CIDEA, PARGC1A and DIO2 were specifically expressed in iPSC-BAs and human fetal BAT (FIG. 7A). Markers for precursors identified in this study such as GATA6 and DPP4 were also detected suggesting the presence of adipocyte precursors in differentiating cultures (FIG. 7A). The top 200 differentially expressed genes in iPSC-BAs corresponded to human WikiPathways related to ‘PPARG signaling pathway’, ‘Estrogen receptor pathway’ and ‘Thermogenesis’ (FIG. 7B). When analyzed using the ProFat database (Cheng et al., 2018), the browning probability of iPSC-BAs was comparable to fetal brown adipocytes (FIG. 7C). Recently, two studies have described the directed differentiation of brown adipocytes from hPSCs (Carobbio et al., 2021; Zhang et al., 2020). Comparison of our iPSC-BAs with the terminally differentiated adipocytes from the two studies—50 day old adipocytes from Zhang et al. (H9-d50) and 25 day old adipocytes from Carobbio et al. (KOLF2-C1-d25) showed that cells differentiated according to our protocol exhibit a higher degree of similarity to the human fetal BAT and brown adipocytes gene expression profile (FIG. 7D, FIG. 18).

### Example 6: Functionality of iPSC-Derived Brown Adipocytes

Next, we sought to investigate the functional potential of iPSC-BAs generated in vitro. Generally, brown adipocytes are characterized by their potential to generate heat in response to a beta-adrenergic stimulus and downstream activation of the cAMP pathway (Wang and Seale, 2016). This activation can be mimicked by treatment with forskolin which activates cAMP signaling (Kriszt et al., 2017; Wang et al., 2020; Zhang et al., 2020). To test the capacity of iPSC-BAs to respond to adrenergic stress, we used forskolin in 40 day-old replated adipogenic cultures. Cells treated with forskolin for 4 hours show increased UCP1 mRNA levels compared to vehicle-treated cells (FIG. 7E). Forskolin treatment also induces lipolysis of stored triglycerides into fatty acids and glycerol. To evaluate the lipolytic activity of the cells, we measured the amount of glycerol released in the medium after forskolin treatment. In comparison to vehicle control treated cells, forskolin-treated cells showed an increased glycerol release indicating that cells underwent lipolysis (FIG. 7F). Brown adipocytes can utilize free fatty acids to induce proton leak and perform thermogenesis. To measure the ability of iPSC-BAs to generate heat, we used ERThermAC, a thermosensitive vital fluorescent dye (Wang et al., 2020). As temperature increases, the cells display lower ERthermAC fluorescence intensity. We preincubated iPSC-BAs with ERThermAC, treated with PBS or forskolin and measured the fluorescence intensity of the dye over time. We observed a reduction in dye intensity in forskolin-treated cells compared to vehicle-treated cells (FIG. 7G). In contrast, no change in intensity was observed for BA precursors in either condition. Overall, our data demonstrate that iPSC-BAs induce UCP1 expression, undergo lipolysis, and generate heat in response to forskolin activation as normal human brown adipocytes.

The process of thermogenesis is driven by the flow of protons through UCP1 in the mitochondrial inner membrane. To examine whether forskolin-induced thermogenesis in iPSC-BAs is coupled to proton leak in mitochondria, we measured oxygen consumption rate (OCR) with a Seahorse metabolic flux analyzer in 40 day-old replated cultures (FIG. 711). Upon treatment with Oligomycin, an ATP synthase inhibitor, the OCR in cells decreased. Interestingly, when iPSC-BAs were treated with forskolin together with Oligomycin, the OCR increased suggesting that proton leak is increased, leading to higher oxygen consumption (FIG. 6D). In contrast, the precursors showed lower basal respiration level and did not show increased OCR upon forskolin treatment. As expected, inhibiting the electron transport chain using Rotenone (mitochondrial complex I inhibitor) or Antimycin (mitochondrial complex III inhibitor), resulted in a drop of OCR (FIG. 711). These analyses support the functionality of human iPSC-BAs differentiated in vitro.

### Example 7. Making UPC-1 Positive Organoids

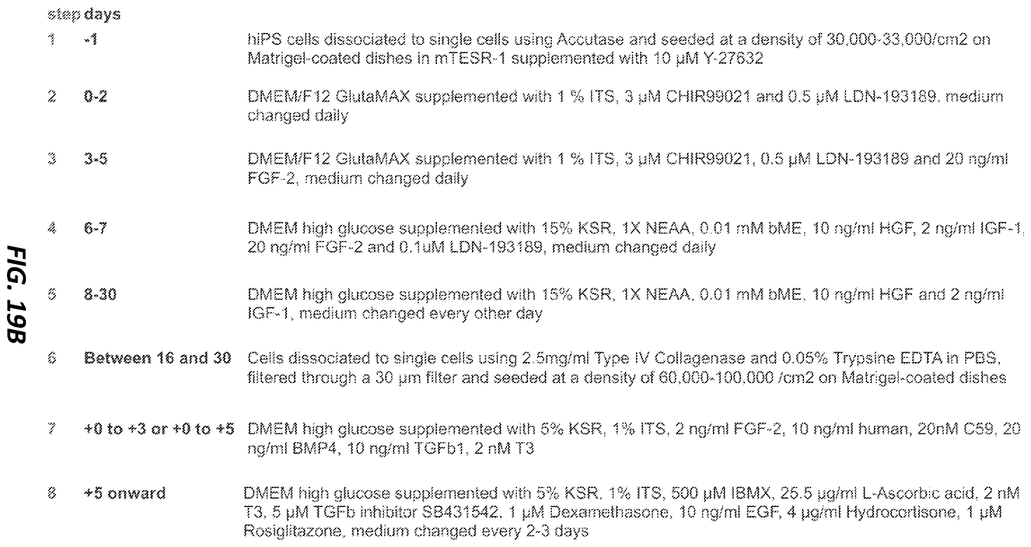

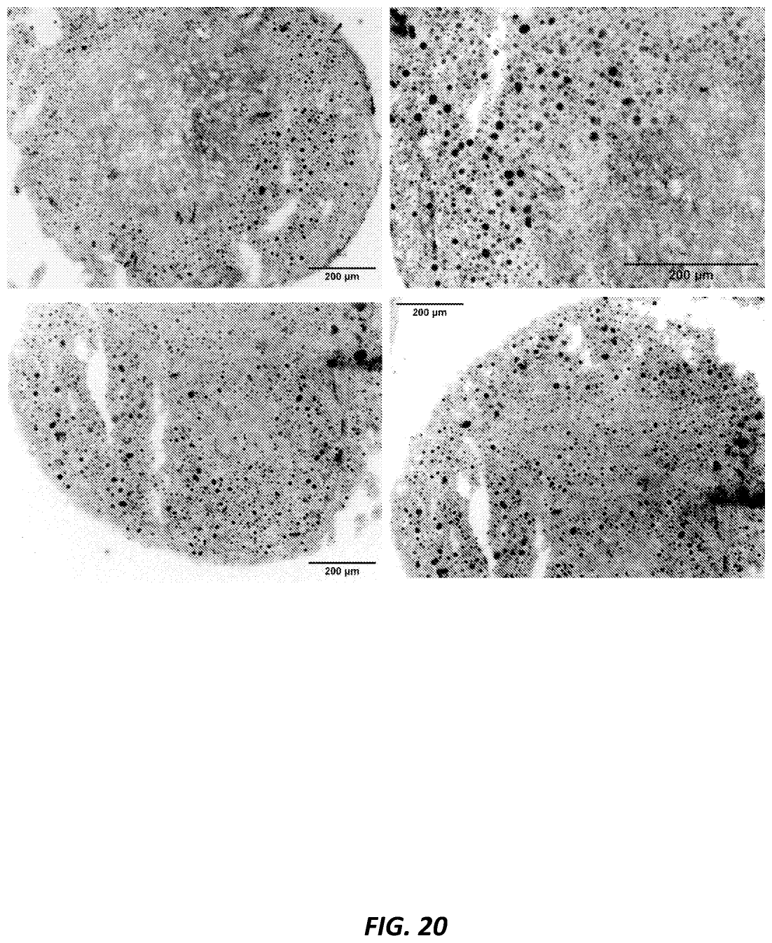

iPSC cultures were dissociated to single cells and aggregated in non-adhesive U-bottom plates to form 3D structures. The structures were cultured in DMEM/F12 GlutaMAX supplemented with 1% ITS, 3 μM CHIR99021, and 0.5 μM LDN-193189 (PSM medium) supplemented with 5% FCS for 11 days to generate PAX3-expressing somite-like structures and dermomyotomal cells. The PAX3-expressing somite-like structures and dermomyotomal cells were then cultured in DMEM high glucose supplemented with 5% KSR, 1% ITS, 2 ng/ml FGF-2, 20 nM C59, 20 ng/ml BMP4, 10 ng/ml TGFb1, 2 nM T3 (adipocyte precursor medium) between 4-12 days. To differentiate the precursors into lipid vesicle containing BA, organoids were cultured in adipogenic differentiation medium (DMEM high glucose supplemented with 5% KSR, 1% ITS, 500 μM IBMX, 25.5 g/ml L-Ascorbic acid, 2 nM T3, 5 μM TGFb inhibitor SB431542, 1 μM Dexamethasone, 10 ng/ml EGF, 4 g/ml Hydrocortisone, 1 μM Rosiglitazone) between 20-40 days. A resulting brown adipose tissue (BAT) organoid was cryosectioned and stained with phalloidin (FIG. 20). Lipid droplets were labeled, and the image is shown in inverted contrast.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Ahfeldt, T., Schinzel, R. T., Lee, YK., Hendrickson, D., Kaplan, A., Lum, D. H., Camahort, R., Xia, F., Shay, J., Rhee, E. P., et al. (2012). Programming human pluripotent stem cells into white and brown adipocytes. Nat Cell Biol 14, 209-219. 10.1038/ncb2411.
Al Tanoury, Z., Rao, J., Tassy, O., Gobert, B., Gapon, S., Garnier, J. M., Wagner, E., Hick, A., Hall, A., Gussoni, E., and Pourquie, O. (2020). Differentiation of the human PAX7-positive myogenic precursors/satellite cell lineage in vitro. Development. 10.1242/dev.187344.
Al Tanoury, Z., Zimmermann, J., Rao, J., Siero, D., McNamara, H. M., Cherrrier, T., and al., E. (2021). Prednisolone rescues Duchenne Muscular Dystrophy phenotypes in human pluripotent stem cells-derived skeletal muscle in vitro PNAS in press.
An, Y, Wang, G., Diao, Y, Long, Y, Fu, X., Weng, M., Zhou, L., Sun, K., Cheung, T. H., Ip, N. Y, et al. (2017). A Molecular Switch Regulating Cell Fate Choice between Muscle Progenitor Cells and Brown Adipocytes. Dev Cell 41, 382-391 e385. 10.1016/j.devcel.2017.04.012.
Angueira, A. R., Sakers, A. P., Holman, C. D., Cheng, L., Arbocco, M. N., Shamsi, F., Lynes, M. D., Shrestha, R., Okada, C., Batmanov, K., et al. (2021). Defining the lineage of thermogenic perivascular adipose tissue. Nat Metab 3, 469-484. 10.1038/s42255-021-00380-0.
Atit, R., Sgaier, S. K., Mohamed, O. A., Taketo, M. M., Dufort, D., Joyner, A. L., Niswander, L., and Conlon, R. A. (2006). Beta-catenin activation is necessary and sufficient to specify the dorsal dermal fate in the mouse. Dev Biol 296, 164-176. 10.1016/j.ydbio.2006.04.449.
Bagchi, D. P., and MacDougald, O. A. (2021). Wnt Signaling: From Mesenchymal Cell Fate to Lipogenesis and Other Mature Adipocyte Functions. Diabetes 70, 1419-1430. 10.2337/dbi20-0015.
Carobbio, S., Guenantin, A. C., Bahri, M., Rodriguez-Fdez, S., Honig, F., Kamzolas, I., Samuelson, I., Long, K., Awad, S., Lukovic, D., et al. (2021). Unraveling the Developmental Roadmap toward Human Brown Adipose Tissue. Stem cell reports 16, 641-655. 10.1016/j.stemcr.2021.01.013.
Chal, J., Al Tanoury, Z., Hestin, M., Gobert, B., Aivio, S., Hick, A., Cherrier, T., Nesmith, A. P., Parker, K. K., and Pourquie, O. (2016). Generation of human muscle fibers and satellite-like cells from human pluripotent stem cells in vitro. Nature protocols 11, 1833-1850. 10.1038/nprot.2016.110.
Chal, J., Oginuma, M., Al Tanoury, Z., Gobert, B., Sumara, O., Hick, A., Bousson, F., Zidouni, Y, Mursch, C., Moncuquet, P., et al. (2015). Differentiation of pluripotent stem cells to muscle fiber to model Duchenne muscular dystrophy. Nat Biotechnol 33, 962-969. 10.1038/nbt.3297.
Chal, J., and Pourquie, O. (2009). Patterning and Differentiation of the Vertebrate Spine. Cold Spring Harbor Monograph The Skeletal System, 41-116.
Chal, J., and Pourquie, O. (2017). Making muscle: skeletal myogenesis in vivo and in vitro. Development 144, 2104-2122. 10.1242/dev.151035.
Chen, H., Albergante, L., Hsu, J. Y, Lareau, C. A., Lo Bosco, G., Guan, J., Zhou, S., Gorban, A. N., Bauer, D. E., Aryee, M. J., et al. (2019). Single-cell trajectories reconstruction, exploration and mapping of omics data with STREAM. Nature communications 10, 1903. 10.1038/s41467-019-09670-4.
Cheng, Y, Jiang, L., Keipert, S., Zhang, S., Hauser, A., Graf, E., Strom, T., Tschop, M., Jastroch, M., and Perocchi, F. (2018). Prediction of Adipose Browning Capacity by Systematic Integration of Transcriptional Profiles. Cell reports 23, 3112-3125. 10.1016/j.celrep.2018.05.021.
Christ, B., Huang, R., and Scaal, M. (2007). Amniote somite derivatives. Dev Dyn.
Cypess, A. M., Lehman, S., Williams, G., Tal, I., Rodman, D., Goldfine, A. B., Kuo, F. C., Palmer, E. L., Tseng, Y H., Doria, A., et al. (2009). Identification and importance of brown adipose tissue in adult humans. The New England journal of medicine 360, 1509-1517. 10.1056/NEJMoa0810780.
Diaz-Cuadros, M., Wagner, D. E., Budjan, C., Hubaud, A., Tarazona, O. A., Donelly, S., Michaut, A., Al Tanoury, Z., Yoshioka-Kobayashi, K., Niino, Y, et al. (2020). In vitro characterization of the human segmentation clock. Nature 580, 113-118. 10.1038/s41586-019-1885-9.
Donati, G., Rognoni, E., Hiratsuka, T., Liakath-Ali, K., Hoste, E., Kar, G., Kayikci, M., Russell, R., Kretzschmar, K., Mulder, K. W., et al. (2017). Wounding induces dedifferentiation of epidermal Gata6(+) cells and acquisition of stem cell properties. Nat Cell Biol 19, 603-613. 10.1038/ncb3532.
Freyer, L., Schroter, C., Saiz, N., Schrode, N., Nowotschin, S., Martinez-Arias, A., and Hadjantonakis, A. K. (2015). A loss-of-function and H2B-Venus transcriptional reporter allele for Gata6 in mice. BMC developmental biology 15, 38. 10.1186/s12861-015-0086-5.
Guenantin, A. C., Briand, N., Capel, E., Dumont, F., Morichon, R., Provost, C., Stillitano, F., Jeziorowska, D., Siffroi, J. P., Hajjar, R. J., et al. (2017). Functional Human Beige Adipocytes From Induced Pluripotent Stem Cells. Diabetes 66, 1470-1478. 10.2337/db16-1107.
Gunawardana, S. C., and Piston, D. W. (2012). Reversal of type 1 diabetes in mice by brown adipose tissue transplant. Diabetes 61, 674-682. 10.2337/db11-0510.
Hafner, A. L., Contet, J., Ravaud, C., Yao, X., Villageois, P., Suknuntha, K., Annab, K., Peraldi, P., Binetruy, B., Slukvin, II, et al. (2016). Brown-like adipose progenitors derived from human induced pluripotent stem cells: Identification of critical pathways governing their adipogenic capacity. Sci Rep 6, 32490. 10.1038/srep32490.
Hasty, P., Bradley, A., Morris, J. H., Edmondson, D. G., Venuti, J. M., Olson, E. N., and Klein, W. H. (1993). Muscle deficiency and neonatal death in mice with a targeted mutation in the myogenin gene. Nature 364, 501-506. 10.1038/364501a0.
Heaton, J. M. (1972). The distribution of brown adipose tissue in the human. J Anat 112, 35-39.
Hutcheson, D. A., Zhao, J., Merrell, A., Haldar, M., and Kardon, G. (2009). Embryonic and fetal limb myogenic cells are derived from developmentally distinct progenitors and have different requirements for beta-catenin. Genes Dev 23, 997-1013. 10.1101/gad.1769009.
Kablar, B., Krastel, K., Tajbakhsh, S., and Rudnicki, M. A. (2003). Myf5 and MyoD activation define independent myogenic compartments during embryonic development. Dev Biol 258, 307-318. 10.1016/s0012-1606(03)00139-8.
Klein, A. M., Mazutis, L., Akartuna, I., Tallapragada, N., Veres, A., Li, V, Peshkin, L., Weitz, D. A., and Kirschner, M. W. (2015). Droplet barcoding for single-cell transcriptomics applied to embryonic stem cells. Cell 161, 1187-1201. 10.1016/j.cell.2015.04.044.
Koutsourakis, M., Langeveld, A., Patient, R., Beddington, R., and Grosveld, F. (1999). The transcription factor GATA6 is essential for early extraembryonic development. Development 126, 723-732.
Kriszt, R., Arai, S., Itoh, H., Lee, M. H., Goralczyk, A. G., Ang, X. M., Cypess, A. M., White, A. P., Shamsi, F., Xue, R., et al. (2017). Optical visualisation of thermogenesis in stimulated single-cell brown adipocytes. Sci Rep 7, 1383. 10.1038/s41598-017-00291-9.
Lepper, C., and Fan, C. M. (2010). Inducible lineage tracing of Pax7-descendant cells reveals embryonic origin of adult satellite cells. Genesis 48, 424-436. 10.1002/dvg.20630.
Lidell, M. E. (2019). Brown Adipose Tissue in Human Infants. Handb Exp Pharmacol 251, 107-123. 10.1007/164_2018_118.
Longo, K. A., Wright, W. S., Kang, S., Gerin, I., Chiang, S. H., Lucas, P. C., Opp, M. R., and MacDougald, O. A. (2004). Wnt10b inhibits development of white and brown adipose tissues. J Biol Chem 279, 35503-35509. 10.1074/jbc.M402937200.
Madisen, L., Zwingman, T. A., Sunkin, S. M., Oh, S. W., Zariwala, H. A., Gu, H., Ng, L. L., Palmiter, R. D., Hawrylycz, M. J., Jones, A. R., et al. (2010). A robust and high-throughput Cre reporting and characterization system for the whole mouse brain. Nat Neurosci 13, 133-140. 10.1038/nn.2467.
Mayeuf-Louchart, A., Lancel, S., Sebti, Y, Pourcet, B., Loyens, A., Delhaye, S., Duhem, C., Beauchamp, J., Ferri, L., Thorel, Q., et al. (2019). Glycogen Dynamics Drives Lipid Droplet Biogenesis during Brown Adipocyte Differentiation. Cell reports 29, 1410-1418 e1416. 10.1016/j.celrep.2019.09.073.
Merklin, R. J. (1974). Growth and distribution of human fetal brown fat. Anat Rec 178, 637-645. 10.1002/ar.1091780311.
Morrisey, E. E., Ip, H. S., Lu, M. M., and Parmacek, M. S. (1996). GATA-6: a zinc finger transcription factor that is expressed in multiple cell lineages derived from lateral mesoderm. Dev Biol 177, 309-322. 10.1006/dbio.1996.0165.
Mullur, R., Liu, Y. Y., and Brent, G. A. (2014). Thyroid hormone regulation of metabolism. Physiol Rev 94, 355-382. 10.1152/physrev.00030.2013.
Muzumdar, M. D., Tasic, B., Miyamichi, K., Li, L., and Luo, L. (2007). A global double-fluorescent Cre reporter mouse. Genesis 45, 593-605. 10.1002/dvg.20335.
Nishio, M., Yoneshiro, T., Nakahara, M., Suzuki, S., Saeki, K., Hasegawa, M., Kawai, Y, Akutsu, H., Umezawa, A., Yasuda, K., et al. (2012). Production of functional classical brown adipocytes from human pluripotent stem cells using specific hemopoietin cocktail without gene transfer. Cell Metab 16, 394-406. 10.1016/j.cmet.2012.08.001.
Ohta, H., and Itoh, N. (2014). Roles of FGFs as Adipokines in Adipose Tissue Development, Remodeling, and Metabolism. Frontiers in endocrinology 5, 18. 10.3389/fendo.2014.00018.
Saito, M., Okamatsu-Ogura, Y, Matsushita, M., Watanabe, K., Yoneshiro, T., Nio-Kobayashi, J., Iwanaga, T., Miyagawa, M., Kameya, T., Nakada, K., et al. (2009). High incidence of metabolically active brown adipose tissue in healthy adult humans: effects of cold exposure and adiposity. Diabetes 58, 1526-1531. 10.2337/db09-0530.
Samuelson, I., and Vidal-Puig, A. (2020). Studying Brown Adipose Tissue in a Human in vitro Context. Frontiers in endocrinology 11, 629. 10.3389/fendo.2020.00629.
Sanchez-Gurmaches, J., and Guertin, D. A. (2014). Adipocytes arise from multiple lineages that are heterogeneously and dynamically distributed. Nature communications 5, 4099. 10.1038/ncomms5099.
Schiebinger, G., Shu, J., Tabaka, M., Cleary, B., Subramanian, V., Solomon, A., Gould, J., Liu, S., Lin, S., Berube, P., et al. (2019). Optimal-Transport Analysis of Single-Cell Gene Expression Identifies Developmental Trajectories in Reprogramming. Cell 176, 928-943 e922. 10.1016/j.cell.2019.01.006.
Schulz, T. J., Huang, P., Huang, T. L., Xue, R., McDougall, L. E., Townsend, K. L., Cypess, A. M., Mishina, Y, Gussoni, E., and Tseng, YH. (2013). Brown-fat paucity due to impaired BMP signalling induces compensatory browning of white fat. Nature 495, 379-383. 10.1038/nature11943.
Schulz, T. J., and Tseng, YH. (2013). Brown adipose tissue: development, metabolism and beyond. Biochem J 453, 167-178. 10.1042/BJ20130457.
Seale, P., Bjork, B., Yang, W., Kajimura, S., Chin, S., Kuang, S., Scime, A., Devarakonda, S., Conroe, H. M., Erdjument-Bromage, H., et al. (2008). PRDM16 controls a brown fat/skeletal muscle switch. Nature 454, 961-967. 10.1038/nature07182.
Seale, P., Kajimura, S., Yang, W., Chin, S., Rohas, L. M., Uldry, M., Tavernier, G., Langin, D., and Spiegelman, B. M. (2007). Transcriptional control of brown fat determination by PRDM16. Cell Metab 6, 38-54. 10.1016/j.cmet.2007.06.001.
Sebo, Z. L., Jeffery, E., Holtrup, B., and Rodeheffer, M. S. (2018). A mesodermal fate map for adipose tissue. Development 145. 10.1242/dev.166801.
Shamsi, F., Piper, M., Ho, L. L., Huang, T. L., Gupta, A., Streets, A., Lynes, M. D., and Tseng, YH. (2021). Vascular smooth muscle-derived Trpv1(+) progenitors are a source of cold-induced thermogenic adipocytes. Nat Metab 3, 485-495. 10.1038/s42255-021-00373-z.
Stanford, K. I., Middelbeek, R. J., Townsend, K. L., An, D., Nygaard, E. B., Hitchcox, K. M., Markan, K. R., Nakano, K., Hirshman, M. F., Tseng, Y H., and Goodyear, L. J. (2013). Brown adipose tissue regulates glucose homeostasis and insulin sensitivity. J Clin Invest 123, 215-223. 10.1172/JC162308.
Takeda, Y, Harada, Y, Yoshikawa, T., and Dai, P. (2017). Direct conversion of human fibroblasts to brown adipocytes by small chemical compounds. Sci Rep 7, 4304. 10.1038/s41598-017-04665-x.
Tseng, Y. H., Kokkotou, E., Schulz, T. J., Huang, T. L., Winnay, J. N., Taniguchi, C. M., Tran, T. T., Suzuki, R., Espinoza, D. O., Yamamoto, Y, et al. (2008). New role of bone morphogenetic protein 7 in brown adipogenesis and energy expenditure. Nature 454, 1000-1004. 10.1038/nature07221.
van Marken Lichtenbelt, W. D., Vanhommerig, J. W., Smulders, N. M., Drossaerts, J. M., Kemerink, G. J., Bouvy, N. D., Schrauwen, P., and Teule, G. J. (2009). Cold-activated brown adipose tissue in healthy men. The New England journal of medicine 360, 1500-1508. 10.1056/NEJMoa0808718.
Virtanen, K. A., Lidell, M. E., Orava, J., Heglind, M., Westergren, R., Niemi, T., Taittonen, M., Laine, J., Savisto, N. J., Enerback, S., and Nuutila, P. (2009). Functional brown adipose tissue in healthy adults. The New England journal of medicine 360, 1518-1525. 10.1056/NEJMoa0808949.
Wang, C. H., Lundh, M., Fu, A., Kriszt, R., Huang, T. L., Lynes, M. D., Leiria, L. O., Shamsi, F., Darcy, J., Greenwood, B. P., et al. (2020). CRISPR-engineered human brown-like adipocytes prevent diet-induced obesity and ameliorate metabolic syndrome in mice. Science translational medicine 12. 10.1126/scitranslmed.aaz8664.
Wang, W., Kissig, M., Rajakumari, S., Huang, L., Lim, H. W., Won, K. J., and Seale, P. (2014). Ebf2 is a selective marker of brown and beige adipogenic precursor cells. Proc Natl Acad Sci USA 111, 14466-14471. 10.1073/pnas.1412685111.
Wang, W., and Seale, P. (2016). Control of brown and beige fat development. Nat Rev Mol Cell Biol 17, 691-702. 10.1038/nrm.2016.96.
Wolf, F. A., Angerer, P., and Theis, F. J. (2018). SCANPY: large-scale single-cell gene expression data analysis. Genome Biol 19, 15. 10.1186/s13059-017-1382-0.
Yao, C. X., Xiong, C. J., Wang, W. P., Yang, F., Zhang, S. F., Wang, T. Q., Wang, S. L., Yu, H. L., Wei, Z. R., and Zang, M. X. (2012). Transcription factor GATA-6 recruits PPARalpha to cooperatively activate Glut4 gene expression. J Mol Biol 415, 143-158. 10.1016/j.jmb.2011.11.011.
Zhang, L., Avery, J., Yin, A., Singh, A. M., Cliff, T. S., Yin, H., and Dalton, S. (2020). Generation of Functional Brown Adipocytes from Human Pluripotent Stem Cells via Progression through a Paraxial Mesoderm State. Cell stem cell 27, 784-797 e711. 10.1016/j.stem.2020.07.013.

1. An in vitro method of generating a Gata6-positive brown adipocyte precursor cell, the method comprising culturing PAX3-positive somatic progenitor cells in a medium comprising effective amounts of each of an FGF signaling pathway activator, a BMP signaling pathway activator, a TGFβ signaling pathway activator, a Wnt signaling pathway inhibitor, and a thyroid hormone receptor activator under conditions and for a time sufficient for the PAX3-positive cells to differentiate into a Gata6-positive brown adipocyte precursor cell.

2. The method of claim 1, wherein the PAX3-positive somatic progenitor cells are generated by a method comprising culturing a pluripotent cell, preferably an induced pluripotent stem cell (iPSC) or embryonic stem (ES) cell in a medium comprising effective amounts of each of an hepatocyte growth factor (HGF) signaling pathway activator, an insulin-like growth factor (IGF) signaling pathway activator, and an fibroblast growth factor (FGF) signaling pathway activator, a Wnt signaling pathway activator, and a bone morphogenetic protein (BMP) signaling pathway inhibitor, to generate a PAX3-positive somatic progenitor cell.

3. The method of claim 1, further comprising culturing the PAX3-positive somatic progenitor cell in a medium comprising an HGF signaling pathway activator and an IGF signaling pathway activator but lacking an FGF signaling pathway activator and a BMP signaling pathway inhibitor for between 8 and 23 days.

4. The methods of claim 3, further comprising dissociating the PAX3-positive somatic progenitor cells after culturing the cells between 8 and 23 days.

5. The method of claim 2, wherein the HGF signaling pathway activator comprises HGF.

6. The method of claim 2, wherein the IGF signaling pathway activator comprises IGF-1.

7. The method of claim 1, wherein the FGF signaling pathway activator comprises at least one of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, and combinations thereof.

8. (canceled)

9. The method of claim 2, wherein the BMP signaling pathway inhibitor comprises at least one of LDN-193189, dorsomorphin, Noggin, Follistatin, Cerberus, and combinations thereof.

10. (canceled)

11. The method of claim 1, wherein the BMP signaling pathway activator comprises at least one of BMP2, BMP4, BMP7, and combinations thereof.

12. The method of claim 1, wherein the Wnt signaling pathway inhibitor comprises at least one of C59, XAV939, IWR-1, JW74, JW55, G007-LK, MSC2504877, benzimidazolone, WNT974, RK-287107, E7499, IWP-L6, GNF-1331, GNF-6231, ETC-131, IWP-29, LGK947, ETC159, ETC-1922159, and combinations thereof.

13. (canceled)

14. The method of claim 1, wherein the TGFβ signaling pathway activator comprises at least one of TGFβ 1, TGFβ2, TGFβ3, and combinations thereof.

15. (canceled)

16. The method of claim 1, wherein the thyroid hormone receptor activator comprises at least one of triiodothyronine (T3), T4, resmetirom, eprotirome, sobetirome, Sob-AM2, VK2809, MB07344, IS25 TG68, and combinations thereof.

17.-20. (canceled)

21. The method of claim 4, wherein dissociating the PAX3-positive somatic progenitor cells comprises applying one or more of Type IV collagenase and trypsin EDTA.

22. (canceled)

23. The method of claim 1, further comprising culturing the Gata6-positive cells in adipogenic differentiation medium to generate brown adipocytes, optionally wherein the adipogenic differentiation medium comprises Dulbecco's Modified Eagle Medium (DMEM) high glucose medium supplemented with serum-free serum replacement, 1× Insulin-Transferrin-Selenium (ITS), isobutylmethylxanthine (IBMX), 1-ascorbic acid, T3, a TGFβ inhibitor, dexamethasone, epidermal growth factor (EGF), hydrocortisone, and rosiglitazone.

24. (canceled)

25. An in vitro method of generating a UCP1-positive, Gata6-positive, and/or Pparg positive brown adipocyte organoid, the method comprising culturing a PAX3-positive somite-like structure in a medium comprising effective amounts of each of an FGF signaling pathway activator, a BMP signaling pathway activator, a TGFβ signaling pathway activator, a Wnt signaling pathway inhibitor, and triiodothyronine (T3), under conditions and for a time sufficient for the PAX3-positive somite-like structure to differentiate into a UCP1-positive, Gata6-positive, and/or Pparg positive brown adipocyte organoid.

26. The method of claim 25, wherein the PAX3-positive somite-like structure is generated by a method comprising culturing a pluripotent cell, preferably an induced pluripotent stem cell (iPSC) or embryonic stem (ES) cell in a medium comprising effective amounts of each of a Wnt activator and a bone morphogenetic protein (BMP) inhibitor, to generate a PAX3-positive somite-like structure.

27. The method of claim 25, wherein the Wnt signaling pathway activator comprises at least one of Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, Wnt-3, R-spondin1, R-spondin2, R-spondin3, R-spondin4, and combinations thereof.

28. The method of claim 25, wherein the Wnt signaling pathway activator comprises CHIR99021.

29. The method of claim 26, wherein the BMP signaling pathway inhibitor comprises at least one of LDN-193189, dorsomorphin, noggin, follistatin, cerberus, and combinations thereof.

30. (canceled)

31. The method of claim 25, wherein the FGF signaling pathway activator comprises at least one of FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22, FGF-23, and combinations thereof.

32. (canceled)

33. The method of claim 25, wherein the BMP signaling pathway activator comprises at least one of BMP2, BMP4, BMP7, and combinations thereof.

34. The method of claim 25, wherein the Wnt signaling pathway inhibitor comprises at least one of C59, XAV939, IWR-1, JW74, JW55, G007-LK, MSC2504877, benzimidazolone, WNT974, RK-287107, E7499, IWP-L6, GNF-1331, GNF-6231, ETC-131, IWP-29, LGK947, ETC159, ETC-1922159, and combinations thereof.

35. (canceled)

36. The method of claim 25, wherein the TGFβ signaling pathway activator comprises at least one of TGFβ 1, TGFβ2, TGFβ3, and combinations thereof.

37. (canceled)

38. The method of claim 25, wherein the thyroid hormone receptor activator comprises at least one of triiodothyronine (T3), T4, resmetirom, eprotirome, sobetirome, Sob-AM2, VK2809, MB07344, IS25 TG68, and combinations thereof.

39. (canceled)

40. The method of claim 25, further comprising culturing the organoids in adipogenic differentiation medium to generate brown adipocyte organoids, optionally wherein the adipogenic differentiation medium comprises Dulbecco's Modified Eagle Medium (DMEM) high glucose medium supplemented with serum-free serum replacement, 1× Insulin-Transferrin-Selenium (ITS), isobutylmethylxanthine (IBMX), 1-ascorbic acid, T3, a TGFβ inhibitor, dexamethasone, epidermal growth factor (EGF), hydrocortisone, and rosiglitazone.

41. (canceled)
